(19) United States
(12) Patent Application Publication
(10) Pub. No.: US 2023/0293712 A1
(43) Pub. Date: Sep. 21, 2023

(54) NOVEL ROR1 ANTIBODY IMMUNOCONJUGATES

(71) Applicant: Velosbio Inc., Rahway, NJ (US)

(72) Inventors: Brian Lannutti, Solana Beach, CA (US); Katti Jessen, San Diego, CA (US); Xin Guo, San Diego, CA (US); Mira Ko, San Diego, CA (US); Jeffry D. Watkins, Encinitas, CA (US); Stephanie Louise Johnson, Llandudno (GB); Colin Martin McKee, Colwyn Bay (GB)

(73) Assignee: Velosbio Inc., Rahway, NJ (US)

(21) Appl. No.: 18/014,820

(22) PCT Filed: Jul. 8, 2021
(86) PCT No.: PCT/US2021/040778

§ 371 Date: Jan. 6, 2023

Related U.S. Application Data
(60) Provisional application No. 63/050,727, filed on Jul. 10, 2020.
(60) Provisional application No. 63/072,665, filed on Aug. 31, 2020.
(60) Provisional application No. 63/105,816, filed on Oct. 26, 2020.

(51) Int. Cl. A61K 47/68; A61P 35/00

(52) U.S. Cl. CPC A61K 47/6849; A61K 47/6803; A61K 47/6889; A61P 35/00

(57) ABSTRACT

Provided herein are immunoconjugates comprising an anti-ROR1 antibody or an antigen-binding fragment thereof and an exatecan moiety or an analog thereof. These immunoconjugates are useful for treating ROR1-expressing cancers.

### BACKGROUND OF THE INVENTION

Receptor tyrosine kinases (RTKs) are key regulators of cell differentiation, proliferation, migration, angiogenesis, and survival. They play an important role in the development and progression of cancer. The receptor tyrosine kinase-like orphan receptor 1 (ROR1) is an evolutionarily-conserved type I membrane protein that belongs to the ROR subfamily and has extracellular domains that contain immunoglobulin (Ig)-like, Frizzled, and Kringle domains. ROR1-deficient mice display a variety of phenotypic defects within the skeletal and urogenital systems, as well as postnatal growth retardation. ROR1 is expressed during embryogenesis and by a variety of different cancers, but not by normal post-partum tissues, and can be considered an onco-embryonic surface antigen. Functional data suggest that ROR1 may function in non-canonical WNT-signaling to promote survival of malignant cells.

ROR1 expression and activation appears to be correlated with features of tumor aggressiveness in models of chronic lymphocytic leukemia (CLL), breast cancer, lung cancer, gastric cancer, and melanoma (Li et al., PLoS One (2010) 5(7):e11859; Gentile et al., Cancer Res. (2011) 71(8):3132-41; Zhang et al., PLoS One (2012) 7(3):e31127; Yamaguchi et al., Cancer Cell (2012) 21(3):348-61; Daneshmanesh et al., Leukemia (2012) 26(6):1348-55; Daneshmanesh et al., Leuk Lymphoma (2013) 54(4):843-50; O'Connell et al., Cancer Discov. (2013) 3(12):1378-93; Hojjat-Farsangi et al., PLoS One (2013) 8(4):e61167; Hojjat-Farsangi et al., PLoS One (2013) 8(10):e78339; Ida et al., Cancer Sci. (2016) 107(2):155-61; and Janovska et al., Clin Cancer Res. (2016) 22(2):459-69). Elevated levels of ROR1 expression in patients and cell lines are associated with genes involved in epithelial-mesenchymal transition (EMT) (Cui et al., Cancer Res. (2013) 73(12):3649-60). In patients with CLL, high levels of ROR1 expression are associated with shorter treatment-free survival and overall survival (OS) (Cui et al., Blood (2016) 128(25):2931-40). Similarly, in patients with ovarian cancer, high ROR1 expression is associated with poor clinical outcomes (Zhang et al., Sci Rep. (2014) 4:5811).

In view of the role of ROR1 in cancer, there is a need for new and improved therapies that target ROR1-positive cancer cells.

### SUMMARY OF THE INVENTION

The present disclosure provides an immunoconjugate having the formula of Ab-(L-D)n, wherein Ab is an antibody or an antigen-binding fragment thereof that specifically binds to human receptor tyrosine kinase like orphan receptor 1 (ROR1); L is a linker; D is exatecan moiety or an analog thereof; and n is an integer from 1 to 10 (e.g., from 1 to 7).

In some embodiments, in an immunoconjugate of the present disclosure, the linker comprises a cleavable moiety.

In some embodiments, the linker is branched. In some embodiments, the linker comprises a tetrapeptide GGFG (SEQ ID NO:55).

In some embodiments, the linker forms a covalent bond with a cysteine residue on the antibody or fragment.

In some embodiments, the linker is covalently bonded to the antibody or antigen-binding fragment at a succinimide, a carbonyl, a cyclooctene, a quaternised vinyl pyridine, or a triazole group of the linker.

In some embodiments, the linker is covalently bonded to the antibody or antigen-binding fragment at a cysteine residue of the antibody or fragment through a succinimide group in the linker, and the succinimide group is linked to a phenyl group through the nitrogen atom.

In some embodiments, the linker comprises a phenyl ring substituted by one or more electron withdrawing groups.

In some embodiments, the linker comprises a phenyl ring substituted by one or more amino groups, one or more PEG chains, and/or one or more glucuronide groups.

In some embodiments, an immunoconjugate of the present disclosure has one of the following structures:

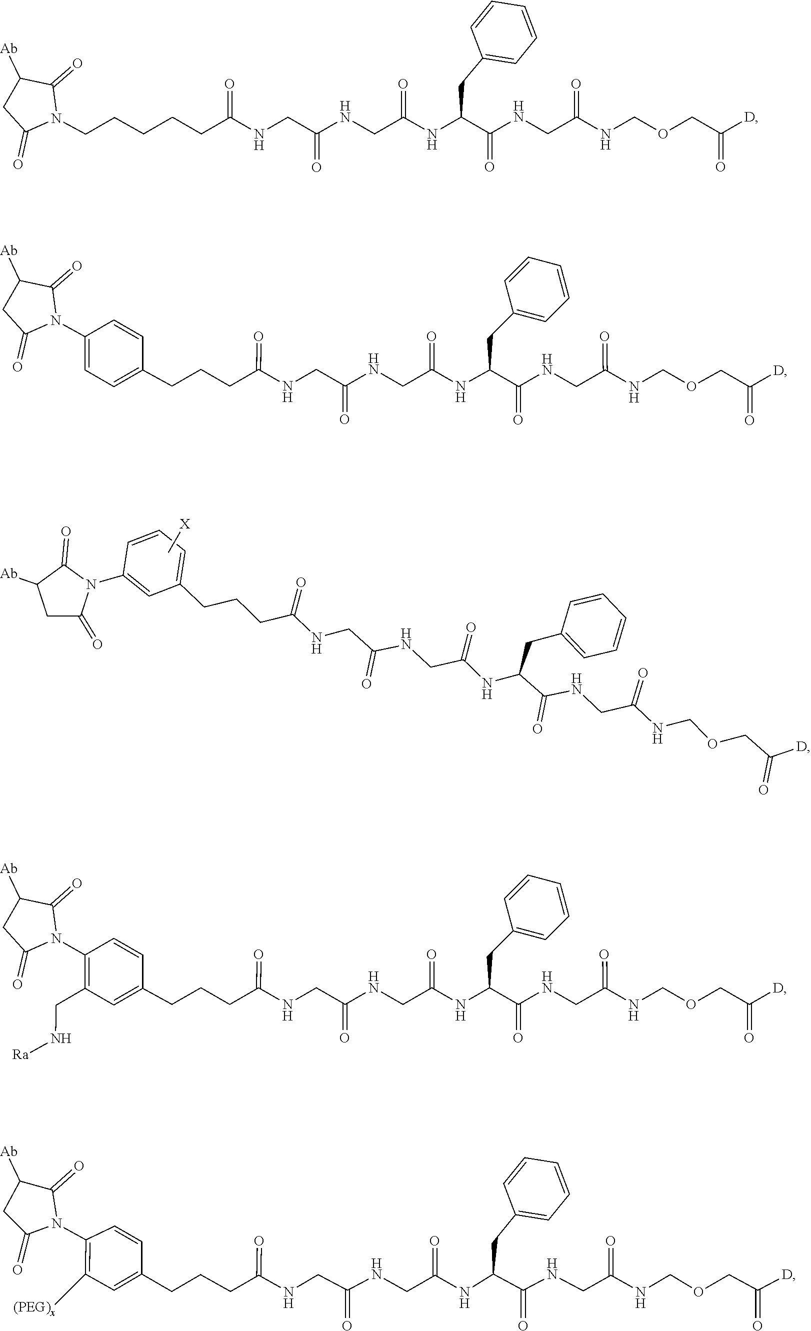

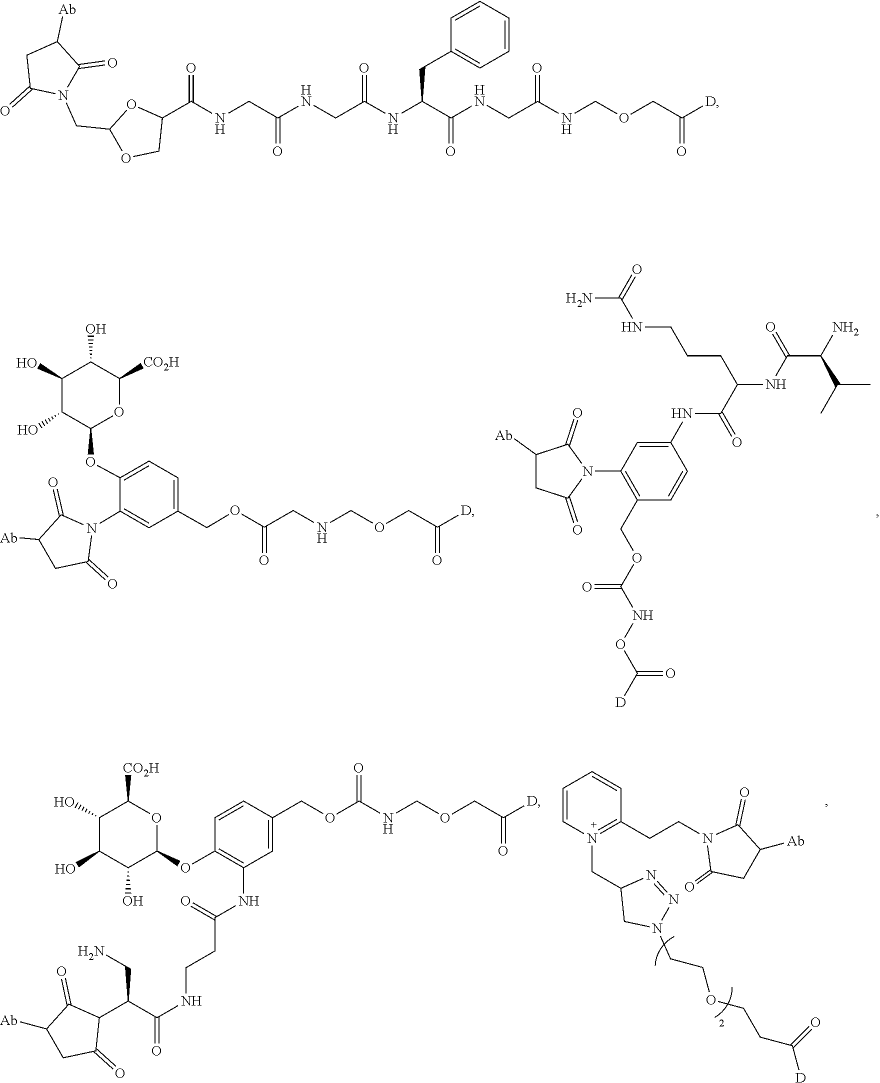

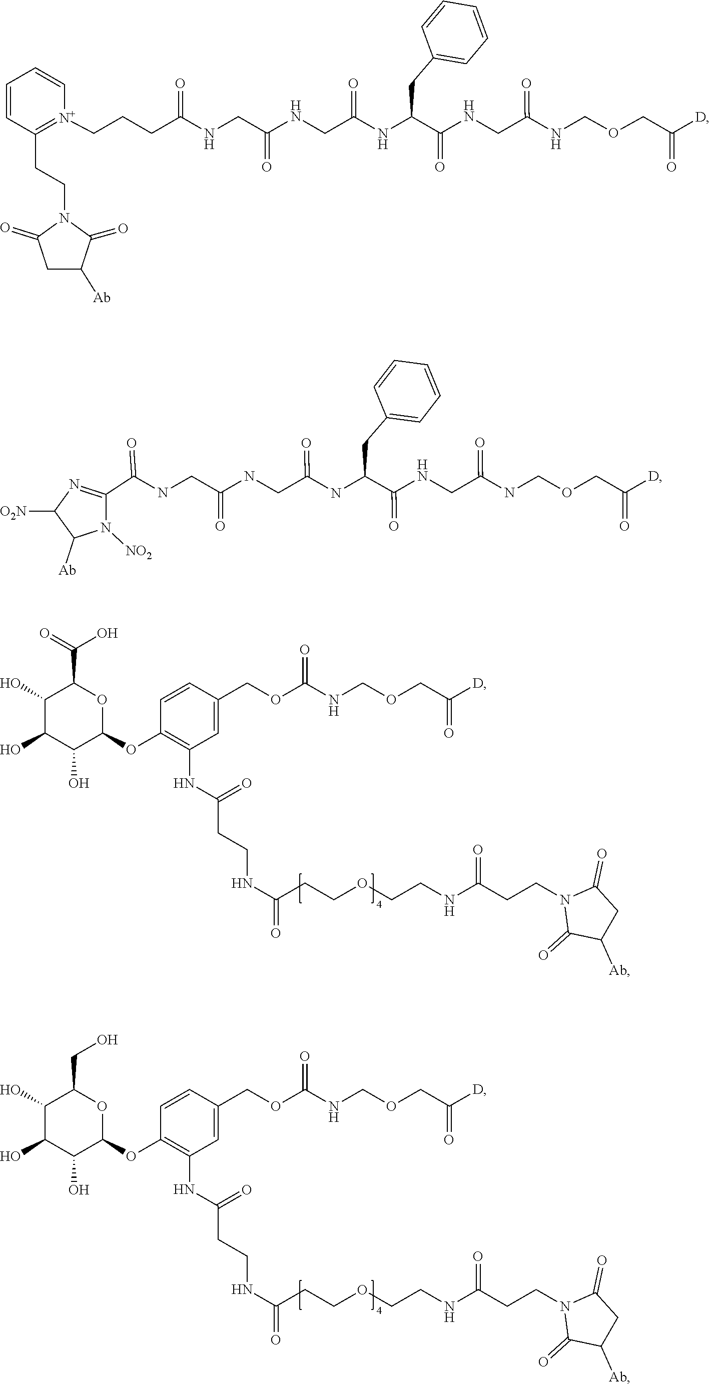

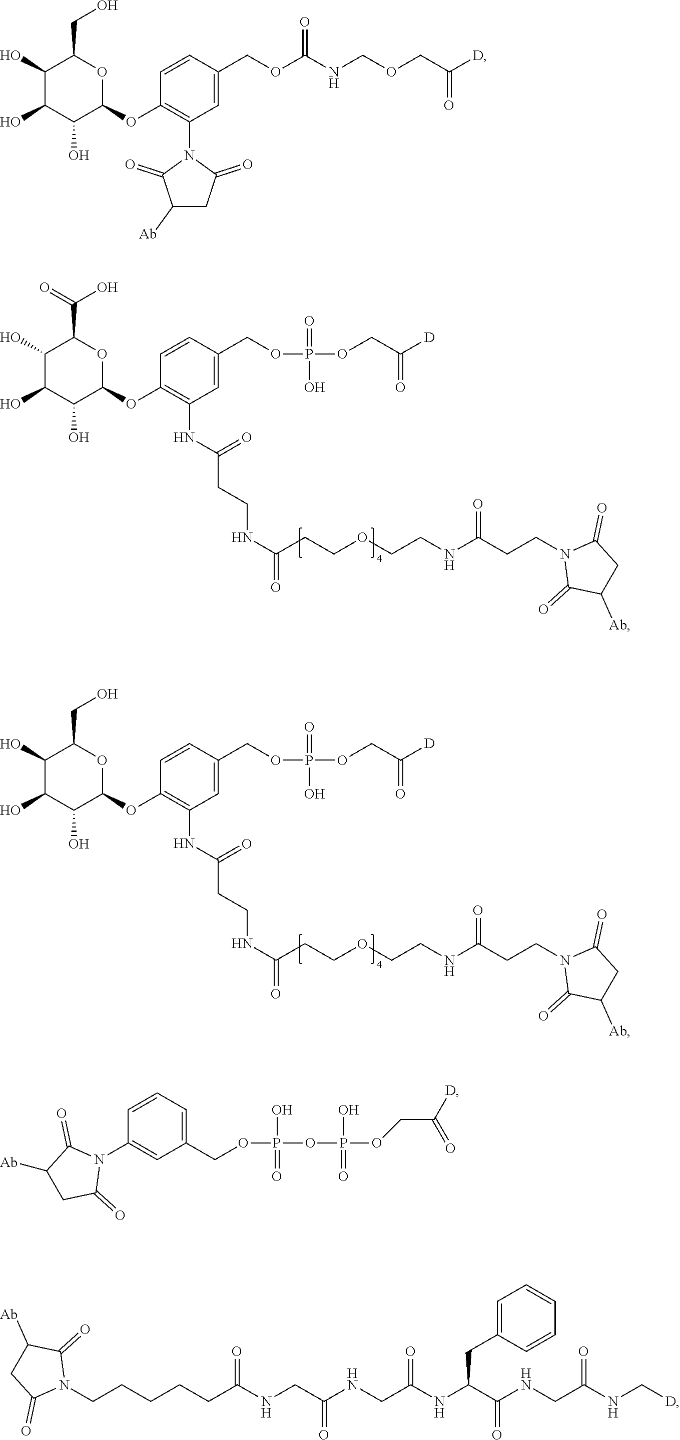

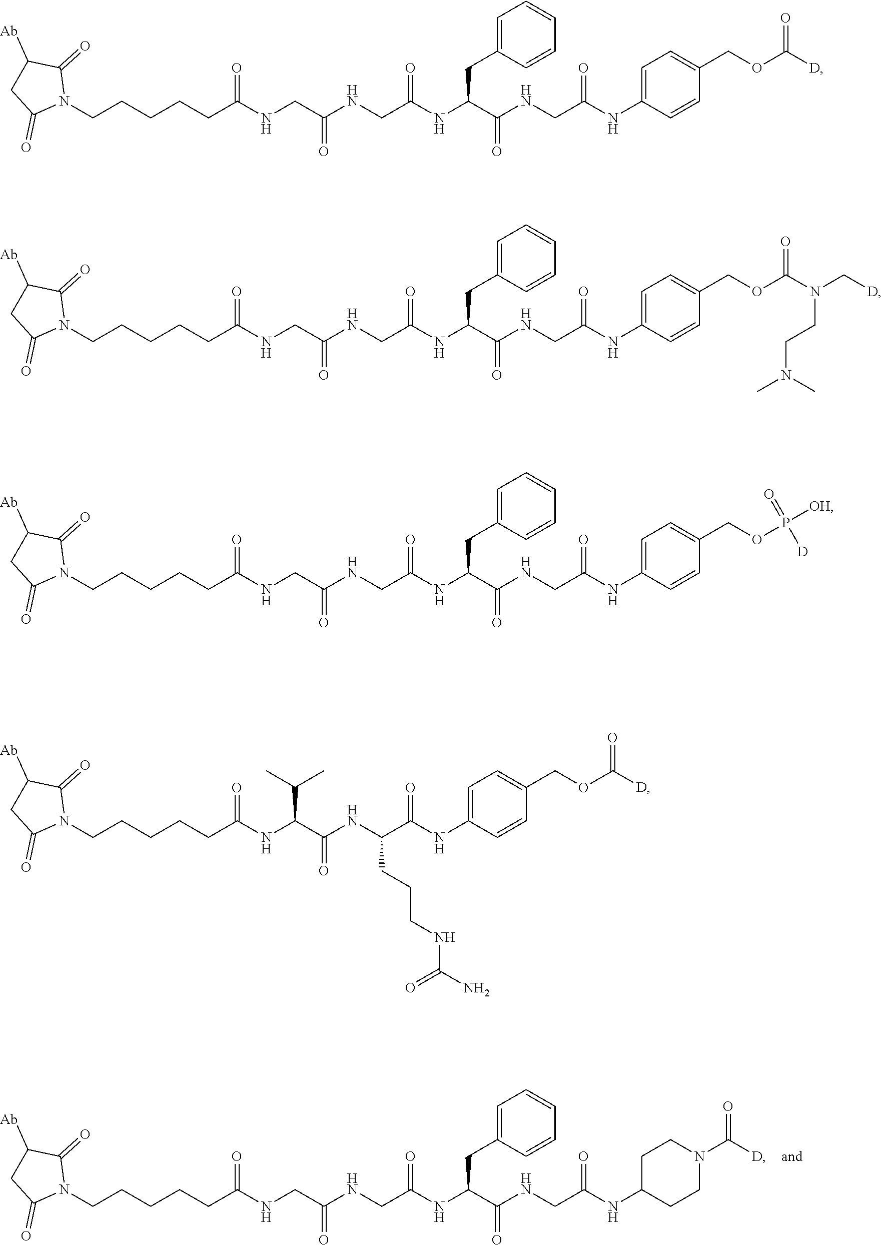

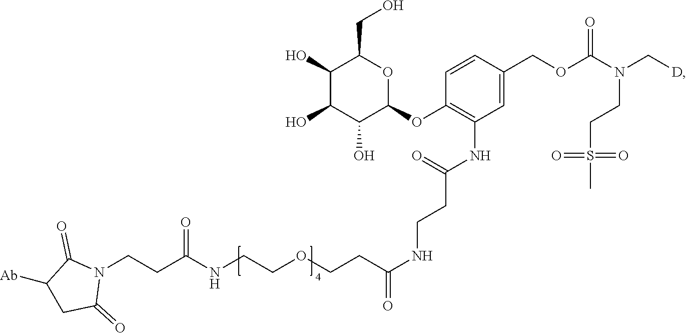

or a pharmaceutically acceptable salt thereof, wherein X is F, Cl, Br, CO₂H, SO₃H, NO₂, CF₃, or CN; Ra is H, methyl, or isopropyl; x is an integer from 1-10, and D is an exatecan moiety or an analog thereof.

In some embodiments, an immunoconjugate of the present disclosure may have one of the following structures:

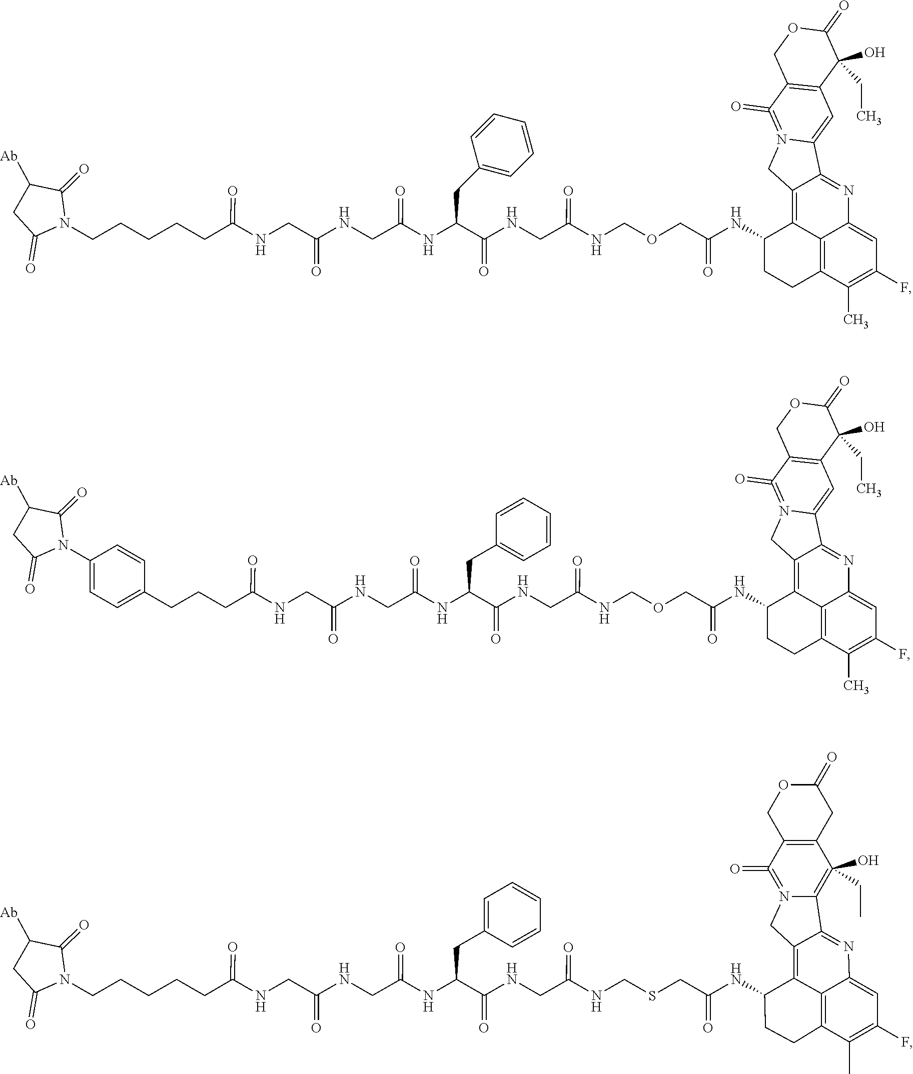

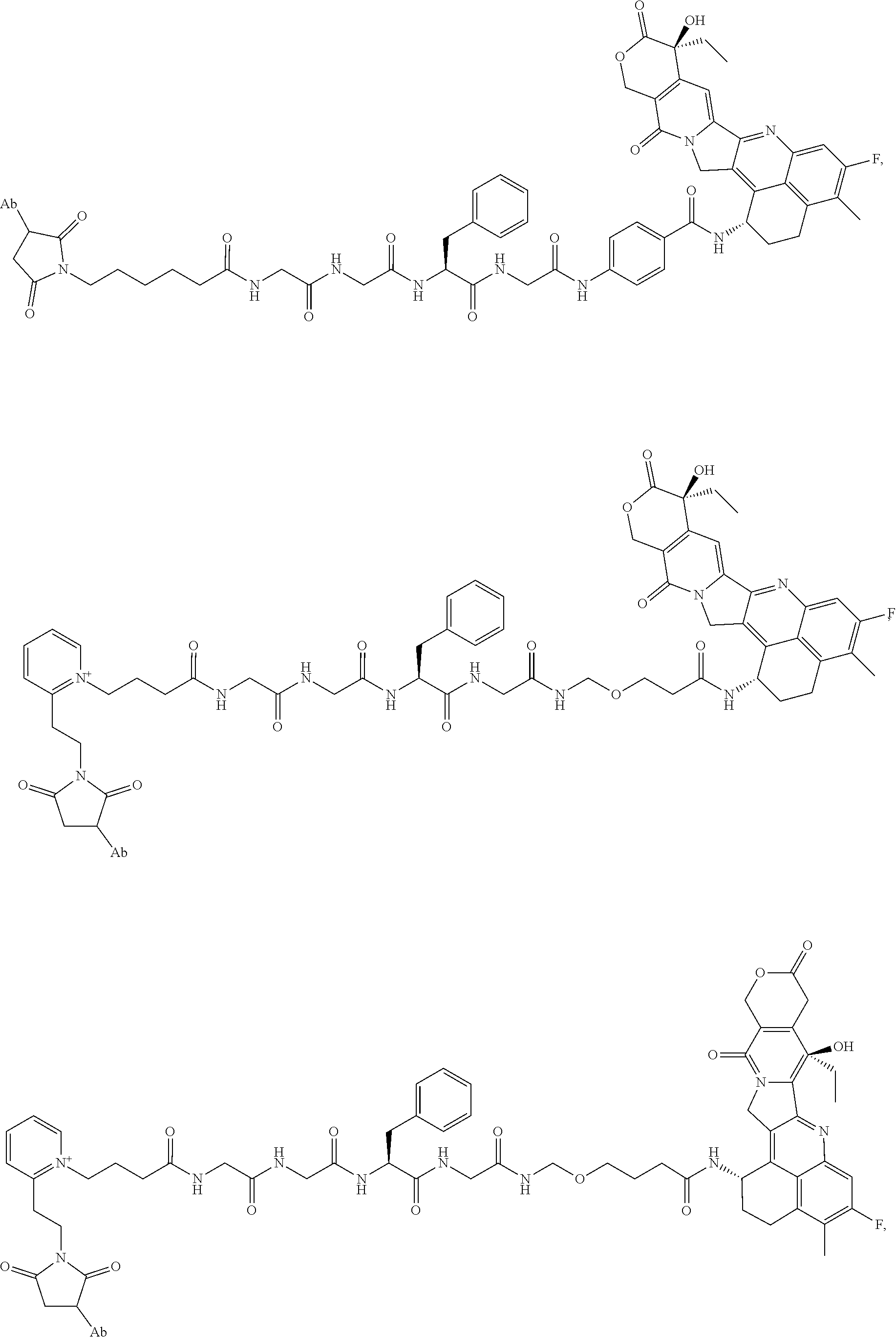

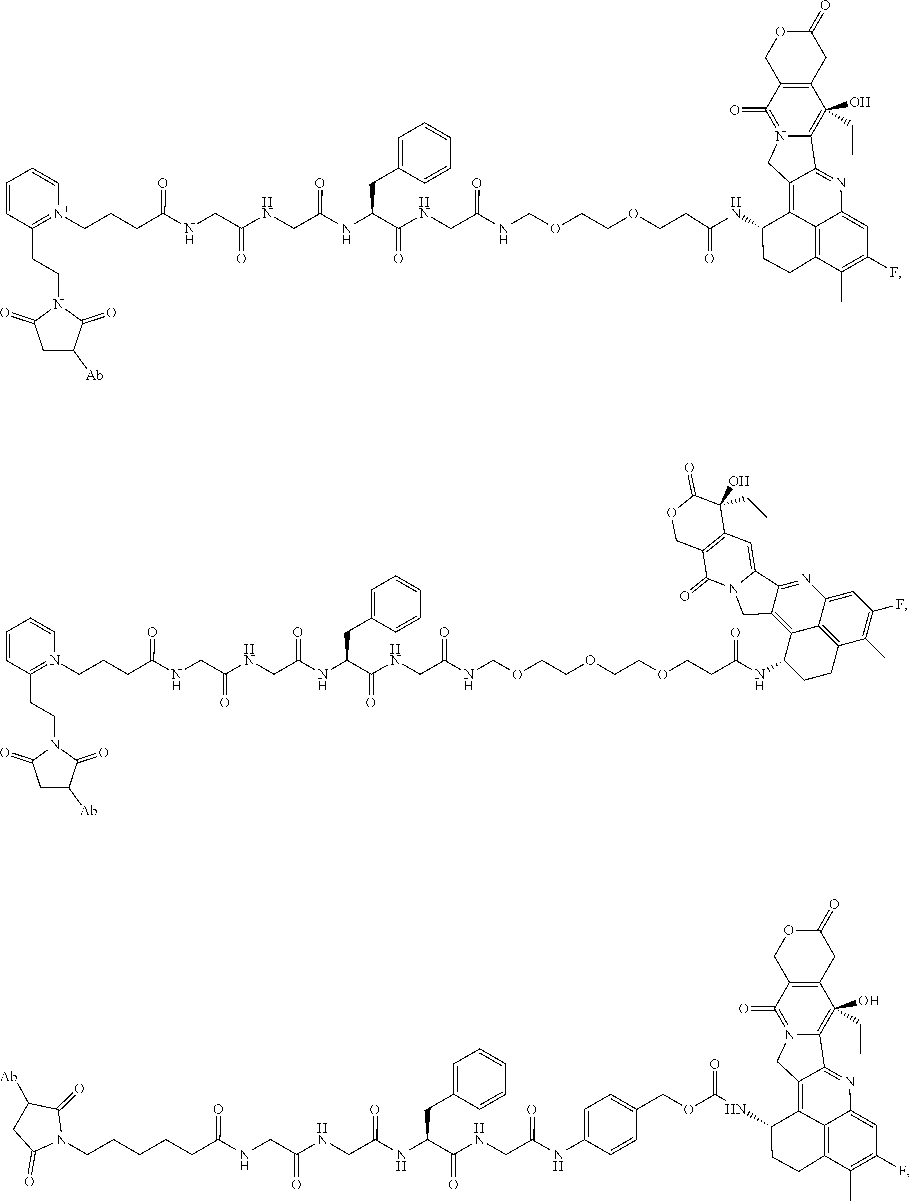

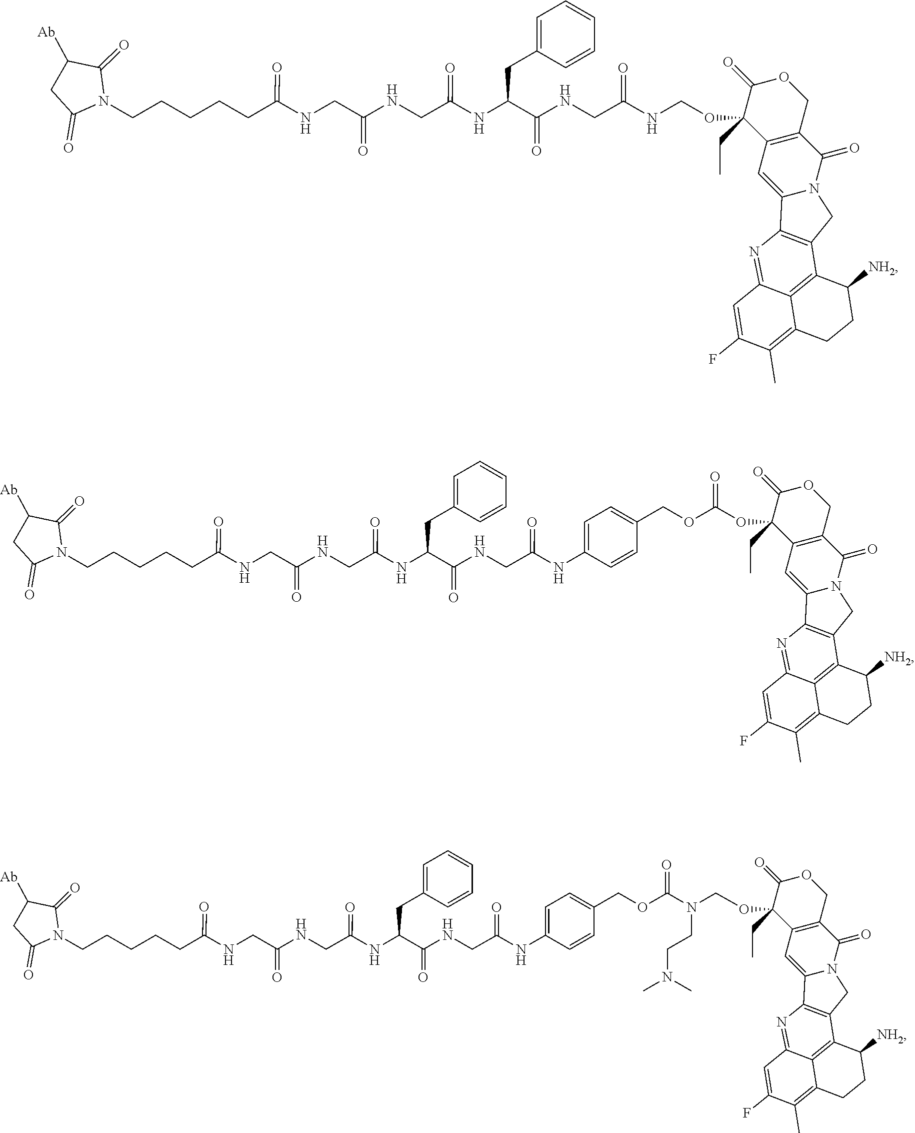

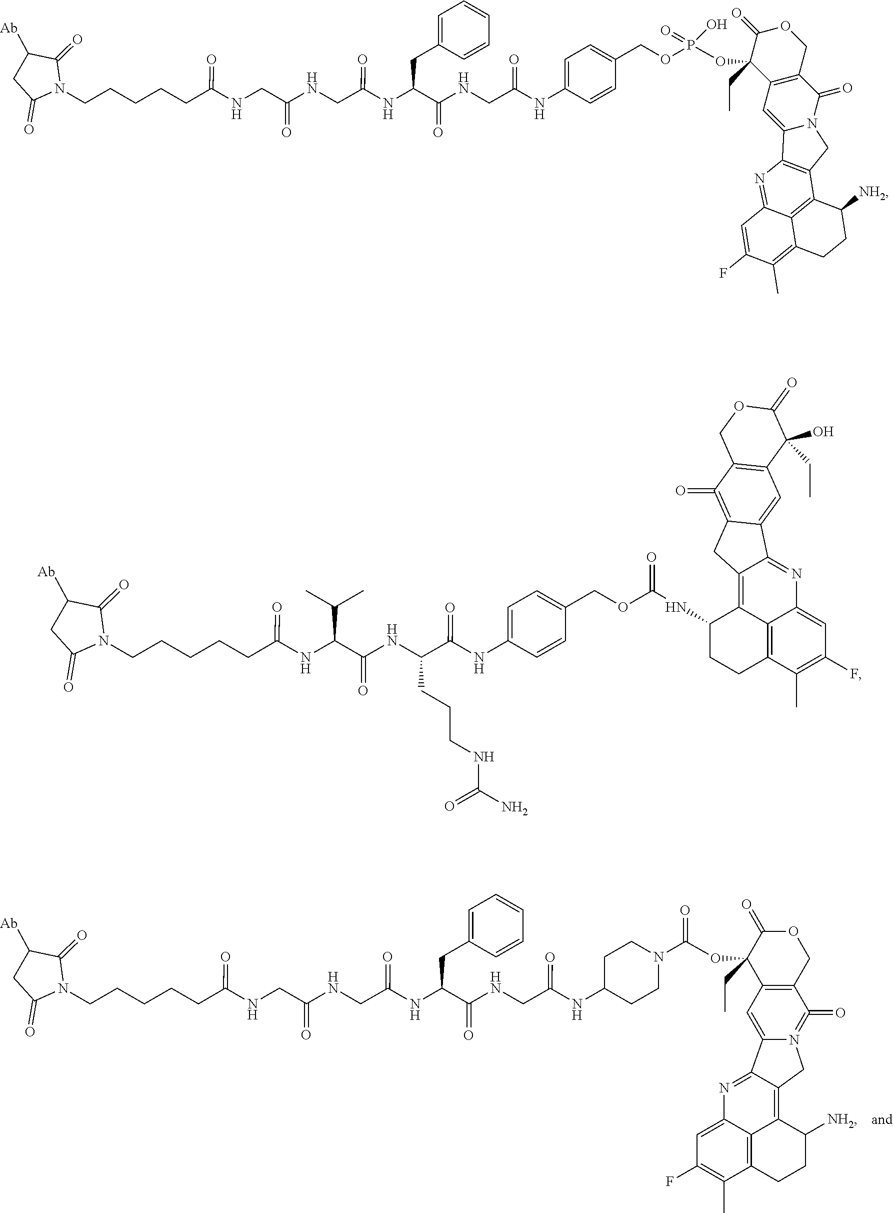

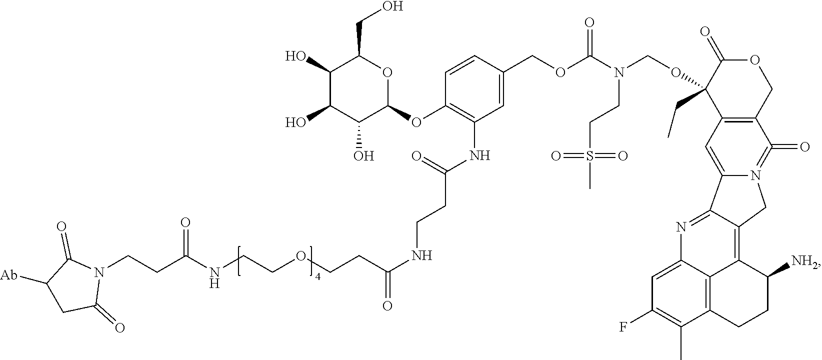

or a pharmaceutically acceptable salt thereof.

In some embodiments, the antibody or fragment binds to the same ROR1 epitope as an antibody comprising the heavy chain and light chain amino acid sequences of SEQ ID NOs: 3 and 4, respectively.

In some embodiments, the antibody comprises (a) the heavy chain complementarity-determining region (CDR) 1-3 (HCDR1-3) in SEQ ID NO: 3 and the light chain CDR1-3 (LCDR1-3) in SEQ ID NO: 4; or (b) the HCDR1-3 in SEQ ID NO: 69 and the LCDR1-3 in SEQ ID NO: 70.

In some embodiments, the heavy chain of the antibody comprises the amino acid sequences of SEQ ID NOs: 7-9, and the light chain of the antibody comprises the amino acid sequences of SEQ ID NOs: 10⁻¹²; the heavy chain of the antibody comprises the amino acid sequences of SEQ ID NOs: 65, 8, and 9, and the light chain of the antibody comprises the amino acid sequences of SEQ ID NOs: 10⁻¹²; the heavy chain of the antibody comprises the amino acid sequences of SEQ ID NOs: 7-9, and the light chain of the antibody comprises the amino acid sequences of SEQ ID NOs: 10, 66, and 12; or the heavy chain of the antibody comprises the amino acid sequences of SEQ ID NOs: 65, 8, and 9, and the light chain of the antibody comprises the amino acid sequences of SEQ ID NOs: 10, 66, and 12.

In some embodiments, the antibody or antigen-binding fragment is humanized.

In some embodiments, the heavy chain variable domain (V_{H}) and light chain variable domain (V_{L}) of the antibody comprise the amino acid sequences of: (a) SEQ ID NOs: 5 and 6, respectively; (b) SEQ ID NOs: 5 and 50, respectively; (c) SEQ ID NOs: 48 and 6, respectively; (d) SEQ ID NOs: 48 and 50, respectively; (e) SEQ ID NOs: 5 and 68, respectively; (f) SEQ ID NOs: 67 and 6, respectively; or (g) SEQ ID NOs: 67 and 68, respectively.

In some embodiments, in an immunoconjugate of the present disclosure, the antibody comprises a human IgG₁ constant region. In some embodiments, the IgG₁ constant region comprises one or more Fc region mutations selected from L234A, L235A, and P329A or P329G.

In some embodiments, the heavy chain and light chain of the antibody comprise the amino acid sequences of: (a) SEQ ID NOs: 3 and 4, respectively; (b) SEQ ID NOs: 3 and 49, respectively; (c) SEQ ID NOs: 47 and 4, respectively; (d) SEQ ID NOs: 47 and 49, respectively; (e) SEQ ID NOs: 69 and 4, respectively; (f) SEQ ID NOs: 3 and 70, respectively; or (g) SEQ ID NOs: 69 and 70, respectively.

In some embodiments, in an immunoconjugate of the present disclosure, the Ab is Fab, F(ab)₂, or scFv.

In some embodiments, the antibody or antigen-binding fragment has one or more of the following properties: (a) facilitates ROR1 internalization in a human cell; (b) binds to human ROR1 with a K_{D} of less than 100 nM; and (c) inhibits growth of ROR1⁺ human cancer cells in vitro with an EC₅₀ of 300 nM or less.

In some embodiments, an immunoconjugate of the present disclosure comprises an anti-ROR1 antibody wherein the V_{H} and V_{L} of the antibody comprise the amino acid sequences of SEQ ID NOs: 5 and 6, respectively, and the immunoconjugate has a structure shown in Table 3 as antibody-drug conjugate (ADC) ADC-U, -V, -W, or -X. In some embodiments, the heavy chain and light chain of the antibody comprise the amino acid sequences of SEQ ID NOs: 3 and 4, respectively. In some embodiments, the heavy chain and light chain of the antibody comprise the amino acid sequences of: (a) SEQ ID NOs: 3 and 70, respectively; (b) SEQ ID NOs: 69 and 4, respectively; or (c) SEQ ID NOs: 69 and 70, respectively.

In another aspect, the present disclosure refers to a pharmaceutical composition comprising an immunoconjugate of the present disclosure and a pharmaceutically acceptable excipient.

In another aspect, the present disclosure refers to a method of treating cancer in a patient in need thereof, comprising administering to the patient a therapeutically effective amount of an immunoconjugate of the present disclosure. In some embodiments, the cancer is heterogeneous for ROR1 expression. In some embodiments, the cancer is a leukemia, a lymphoma, or a solid tumor, optionally wherein the cancer is chronic lymphocytic leukemia, acute lymphoblastic leukemia, acute myeloid leukemia, small lymphocytic leukemia, follicular lymphoma, T cell non-Hodgkin lymphoma, lymphoplasmacytoid lymphoma, mantle cell lymphoma, diffuse large B-cell lymphoma, Burkitt's lymphoma, Waldenström macroglobulinemia, marginal zone lymphoma, or a non-Hodgkin lymphoma that has undergone Richter's transformation; or the cancer is non-small cell lung cancer, sarcoma, ovarian cancer, or breast cancer, optionally wherein the breast cancer is triple negative breast cancer.

In another aspect, the present disclosure refers to an immunoconjugate of the present disclosure, or a pharmaceutical composition of the present disclosure, for use in treating cancer in a method recited in the present disclosure.

In another aspect, the present disclosure refers to the use of an immunoconjugate of the present disclosure for the manufacture of a medicament for treating cancer in a method recited in the present disclosure.

Provided herein also are articles of manufactures, such as kits, comprising an immunoconjugate of the present disclosure.

Other features, objectives, and advantages of the invention are apparent in the detailed description that follows. It should be understood, however, that the detailed description, while indicating embodiments and aspects of the invention, is given by way of illustration only, not limitation. Various changes and modifications within the scope of the invention will become apparent to those skilled in the art from the detailed description.

### BRIEF DESCRIPTION OF THE DRAWINGS

FIG. 1 is a graph illustrating the stability of ADC-U (open symbols) and ADC-W (closed symbols) in human (H, squares), cynomolgus monkey (C, circles), and mouse (M, triangles) plasma at 37° C.

FIG. 2 is a graph illustrating the stability of ADC-V (open symbols) and ADC-X (closed symbols) in human (H, squares), cynomolgus monkey (C, circles), and mouse (M, triangles) plasma at 37° C.

FIG. 3 is a graph illustrating tumor growth inhibition in an MCL xenograft model upon treatment with vehicle, 2.5- and 5 mg/kg ADC-U or ADC-W intravenously (IV) weekly for three weeks.

FIG. 4 is a graph illustrating tumor growth inhibition in an MCL xenograft model upon treatment with vehicle, 2.5- and 5 mg/kg ADC-V or ADC-X intravenously (IV) weekly for three weeks.

### DETAILED DESCRIPTION OF THE INVENTION

The present invention provides immunoconjugates comprising an anti-ROR1 antibody or an antigen-binding fragment thereof, and exatecan or an analog thereof as a cytotoxic drug moiety. An immunoconjugate of the present invention has the formula Ab-(L-D)n, wherein Ab is an antibody or an antigen-binding fragment thereof that specifically binds to the ROR1 protein; L is a linker; D is exatecan or an analog thereof; and n is an integer from 1 to 10. In the formula, the dash “-” denotes a covalent or non-covalent bond. The antibody or fragment includes, but is not limited to, an antibody or antibody fragment that competes with antibody D10 or Ab1 for binding to human ROR1, or binds to the same epitope as D10 or Ab1. The immunoconjugates of the present invention may be used to treat a variety of cancers such as ROR1-positive cancers.

### I. Immunoconjugates

An “antibody-drug conjugate,” or “ADC,” or “immunoconjugate” refers to an antibody molecule, or an antigen-binding fragment thereof, that is covalently or non-covalently bonded, with or without a linker, to one or more biologically active molecule(s). The present immunoconjugates comprise antibodies or fragments thereof that are specific for human ROR1 and can thus serve as excellent targeting moieties for delivering the conjugated payloads to ROR1-positive cells. In some embodiments, a ROR1 immunoconjugate provided herein has an equilibrium dissociation constant (K_{D}) of about 100, 50, 40, 30, 20, 10, 5, 2, 1, 0.5, 0.1, 0.05, 0.01, or 0.001 nM or less (e.g., 10⁻⁸ M or less, from 10⁻⁸ M to 10⁻¹³ M, or from 10⁻⁹ M to 10⁻¹³ M) for human ROR1. K_{D} can be measured by any suitable assay, such as surface plasmon resonance assays (e.g., using a BIACORE®-2000 or a BIACORE®-3000. In some embodiments, a ROR1 immunoconjugate provided herein inhibits growth of ROR1⁺ human cancer cells in vitro with an EC₅₀ of about 1000, 900, 800, 700, 600, 500, 400, 300, 200, 150, 100, 75, 50, 40, or 30 nM or less (e.g., 256 nM or less). As used herein, an antibody is said to bind specifically to an antigen when it binds to the antigen with a K_{D} of 100 nM or less, such as 10 nM or less (e.g., 1-5 nM), as determined by, e.g., surface plasmon resonance or Bio-Layer Interferometry.

Embodiments of the antibody or fragment thereof, the linker, and the drug moiety used in the immunoconjugates are described in further detail below.

1. Types and Structures of Antibodies

The term “antibody” is used herein in the broadest sense and includes monoclonal antibodies, such as intact antibodies and functional (antigen-binding) fragments thereof. The term encompasses genetically engineered and/or otherwise modified forms of immunoglobulins, such as chimeric antibodies, fully human antibodies, humanized antibodies, multi-specific (e.g., bispecific) antibodies, diabodies, triabodies, and tetrabodies, tandem di-scFv, and tandem tri-scFv. Unless otherwise indicated, the term encompasses intact or full-length antibodies, including antibodies of any class or subclass (e.g., IgG and sub-classes thereof such as IgG₁, IgG₂, IgG₃, and IgG₄; IgM; IgE; IgA; and IgD), as well as antibody fragments.

An antibody may include a heavy chain (or a polypeptide sequence derived therefrom) and a light chain (or a polypeptide sequence derived therefrom). The term “variable region” or “variable domain” refers to the domain of an antibody heavy or light chain that is involved in the antibody's binding to an antigen. The variable domains of the heavy chain and light chain (V_{H} and V_{L}, respectively) of a native antibody generally have similar structures, with each domain comprising four conserved framework regions and three complementarity-determining regions. A single V_{H} or V_{L} domain may sometimes be sufficient to confer all or a majority of the antigen-binding specificity of an antibody. Furthermore, antibodies that bind a particular antigen may be isolated by using a V_{H} or V_{L} domain from an antibody that binds the antigen to screen a library of complementary V_{L} or V_{H} domains, respectively. See, e.g., Portolano et al., J Immunol. (1993) 150:880-7; Clarkson et al., Nature (1991) 352:624-8.

An antigen-binding fragment of a full-length antibody may be used in making an immunoconjugate of the present invention. Examples of antibody fragments include, but are not limited to, Fv, Fab, Fab′, Fab′-SH, F(ab′)2; recombinant IgG (rIgG) fragments; diabodies; linear antibodies; single-chain antibody molecules (e.g., scFv or sFv); single domain antibodies (e.g., sdAb, sdFv, nanobodies); and multi-specific antibodies formed from antibody fragments. In certain embodiments, the fragments are single-chain antibody fragments comprising a variable heavy chain region and/or a variable light chain region, such as scFvs.

2. Exemplary ROR1 Antibodies

An immunoconjugate of the invention comprises an antibody or an antigen-binding fragment thereof that specifically binds to ROR1, e.g., human ROR1. The antibody or fragment binds to an extracellular portion of the ROR1 protein such as an epitope in one or more of the immunoglobulin (Ig)-like, Frizzled, and Kringle domains of the ROR1 protein. In certain embodiments, the ROR1-binding antibody or fragment binds to an amino acid sequence of ROR1 shown in SEQ ID NO: 1 or 2 (not including the terminal cysteine, which is added for convenience of conjugation) and can be internalized by a ROR1⁺ cell; examples of such an antibody are murine antibodies D10 and 99961. See U.S. Pat. Nos. 9,217,040 and 9,758,591, the disclosures of which are incorporated by reference herein in their entirety. In certain embodiments, the antibody or fragment competes with D10 or 99961 for binding to human ROR1. Amino acid sequences of exemplary anti-ROR1 antibodies used in the immunoconjugates of the invention are shown in Table 1 below, where Ab1-Ab7 are humanized variants of antibody 99961.

TABLE 1
SEQ ID NOs of Exemplary Anti-ROR1 Antibodies

| Ab | HCDR1 | HCDR2 | HCDR3 | VH | HC | LCDR1 | LCDR2 | LCDR3 | VL | LC |
|---|---|---|---|---|---|---|---|---|---|---|
| 99961 | 7 | 8 | 9 | 45 | — | 10 | 11 | 12 | 46 | — |
| Ab1 | 7 | 8 | 9 | 5 | 3 | 10 | 11 | 12 | 6 | 4 |
| Ab2 | 7 | 8 | 9 | 5 | 3 | 10 | 11 | 12 | 50 | 49 |
| Ab3 | 7 | 8 | 9 | 48 | 47 | 10 | 11 | 12 | 6 | 4 |
| Ab4 | 7 | 8 | 9 | 48 | 47 | 10 | 11 | 12 | 50 | 49 |
| Ab5 | 65 | 8 | 9 | 67 | 69 | 10 | 11 | 12 | 6 | 4 |
| Ab6 | 7 | 8 | 9 | 5 | 3 | 10 | 66 | 12 | 68 | 70 |
| Ab7 | 65 | 8 | 9 | 67 | 69 | 10 | 66 | 12 | 68 | 70 |
| D10 | 27 | 28 | 29 | 25 | — | 30 | 31 | 32 | 26 | — |

In some embodiments, the antibody or antibody fragment in the immunoconjugate specifically binds human ROR1, and its heavy and light chains respectively comprise:
a) the heavy chain CDR1-3 (HCDR1-3) amino acid sequences in SEQ ID NO: 3, and the light chain CDR1-3 (LCDR1-3) amino acid sequences in SEQ ID NO: 4;
b) HCDR1-3 comprising the amino acid sequences of SEQ ID NO: 7-9, respectively, and LCDR1-3 comprising the amino acid sequences of SEQ ID NOs: 10⁻¹², respectively;
c) the HCDR1-3 amino acid sequences in SEQ ID NO: 13-15, and the LCDR1-3 amino acid sequences in SEQ ID NOs: 16-18;
d) HCDR1-3 comprising the amino acid sequences of SEQ ID NO: 27-29, respectively, and LCDR1-3 comprising the amino acid sequences of SEQ ID NOs: 30-32, respectively;
e) HCDR1-3 comprising the amino acid sequences of SEQ ID NO: 37-39, respectively, and LCDR1-3 comprising the amino acid sequences of SEQ ID NOs: 40-42, respectively;
f) HCDR1-3 comprising the amino acid sequences of SEQ ID NO: 65, 8, and 9, respectively, and LCDR1-3 comprising the amino acid sequences of SEQ ID NOs: 10⁻¹², respectively;
g) HCDR1-3 comprising the amino acid sequences of SEQ ID NO: 7-9, respectively, and LCDR1-3 comprising the amino acid sequences of SEQ ID NO: 10, 66, and 12, respectively;
h) HCDR1-3 comprising the amino acid sequences of SEQ ID NO: 65, 8, and 9, respectively, and LCDR1-3 comprising the amino acid sequences of SEQ ID NOs: 10, 66, and 12, respectively;
i) HCDR1-3 comprising residues 26-33, 51-58, and 97-105 of SEQ ID NO: 5 or 67, respectively, and LCDR1-3 comprising residues 27-32, 50-52, and 89-97 of SEQ ID NO: 6 or 68, respectively;
j) HCDR1-3 comprising residues 26-32, 52-57, and 99-105 of SEQ ID NO: 5 or 67, respectively, and LCDR1-3 comprising residues 24-34, 50-56, and 89-97 of SEQ ID NO: 6 or 68, respectively;
k) HCDR1-3 comprising residues 31-35, 50-66, and 99-105 of SEQ ID NO: 5 or 67, respectively, and LCDR1-3 comprising residues 24-34, 50-56, and 89-97 of SEQ ID NO: 6 or 68, respectively;
l) HCDR1-3 comprising residues 26-32, 52-57, and 99-105 of SEQ ID NO: 5 or 67, respectively, and LCDR1-3 comprising residues 27-32, 50-52, and 89-97 of SEQ ID NO: 6 or 68, respectively; or
m) HCDR1-3 comprising residues 31-35, 52-57, and 99-105 of SEQ ID NO: 5 or 67, respectively, and LCDR1-3 comprising residues 27-32, 50-52, and 89-97 of SEQ ID NO: 6 or 68, respectively.

In some embodiments, the antibody or fragment is humanized, or chimeric with human constant regions. In further embodiments, the antibody or fragment may comprise a human IgG₁, IgG₂, IgG₃, or IgG₄ constant region and optionally a human κ constant region.

In certain embodiments, the immunoconjugate of the invention comprises an anti-ROR1 antibody, or an antigen-binding fragment thereof, wherein the antibody comprises:
a) a heavy chain variable domain or region (V_{H}) comprising an amino acid sequence at least 80%, 85%, 90%, 91%, 92%, 93%, 94%, 95%, 96%, 97%, 98%, or 99% (e.g., at least 90%) identical to that of SEQ ID NO: 5, and a light chain variable domain or region (V_{L}) comprising an amino acid sequence at least 80%, 85%, 90%, 91%, 92%, 93%, 94%, 95%, 96%, 97%, 98%, or 99% (e.g., at least 90%) identical to that of SEQ ID NO: 6;
b) a V_{H} and a V_{L} comprising the amino acid sequences of SEQ ID NOs: 5 and 6, respectively;
c) a heavy chain (HC) comprising an amino acid sequence at least 80%, 85%, 90%, 91%, 92%, 93%, 94%, 95%, 96%, 97%, 98%, or 99% (e.g., at least 90%) identical to that of SEQ ID NO: 3 and a light chain (LC) comprising an amino acid sequence 80%, 85%, 90%, 91%, 92%, 93%, 94%, 95%, 96%, 97%, 98%, or 99% (e.g., at least 90%) identical to that of SEQ ID NO: 4; or
d) an HC and an LC comprising the amino acid sequences of SEQ ID NOs: 3 and 4, respectively.

In certain embodiments, the immunoconjugate of the invention comprises an anti-ROR1 antibody, or an antigen-binding fragment thereof, wherein the antibody comprises:
a) a heavy chain variable domain or region (V_{H}) comprising an amino acid sequence at least 80%, 85%, 90%, 91%, 92%, 93%, 94%, 95%, 96%, 97%, 98%, or 99% (e.g., at least 90%) identical to that of SEQ ID NO: 67, and a light chain variable domain or region (V_{L}) comprising an amino acid sequence at least 80%, 85%, 90%, 91%, 92%, 93%, 94%, 95%, 96%, 97%, 98%, or 99% (e.g., at least 90%) identical to that of SEQ ID NO: 6;
b) a V_{H} and a V_{L} comprising the amino acid sequences of SEQ ID NOs: 67 and 6, respectively;
c) a heavy chain (HC) comprising an amino acid sequence at least 80%, 85%, 90%, 91%, 92%, 93%, 94%, 95%, 96%, 97%, 98%, or 99% (e.g., at least 90%) identical to that of SEQ ID NO: 69 and a light chain (LC) comprising an amino acid sequence 80%, 85%, 90%, 91%, 92%, 93%, 94%, 95%, 96%, 97%, 98%, or 99% (e.g., at least 90%) identical to that of SEQ ID NO: 4; or
d) an HC and an LC comprising the amino acid sequences of SEQ ID NOs: 69 and 4, respectively.

In certain embodiments, the immunoconjugate of the invention comprises an anti-ROR1 antibody, or an antigen-binding fragment thereof, wherein the antibody comprises:
a) a heavy chain variable domain or region (V_{H}) comprising an amino acid sequence at least 80%, 85%, 90%, 91%, 92%, 93%, 94%, 95%, 96%, 97%, 98%, or 99% (e.g., at least 90%) identical to that of SEQ ID NO: 5, and a light chain variable domain or region (V_{L}) comprising an amino acid sequence at least 80%, 85%, 90%, 91%, 92%, 93%, 94%, 95%, 96%, 97%, 98%, or 99% (e.g., at least 90%) identical to that of SEQ ID NO: 68;
b) a V_{H} and a V_{L} comprising the amino acid sequences of SEQ ID NOs: 5 and 68, respectively;
c) a heavy chain (HC) comprising an amino acid sequence at least 80%, 85%, 90%, 91%, 92%, 93%, 94%, 95%, 96%, 97%, 98%, or 99% (e.g., at least 90%) identical to that of SEQ ID NO: 3 and a light chain (LC) comprising an amino acid sequence 80%, 85%, 90%, 91%, 92%, 93%, 94%, 95%, 96%, 97%, 98%, or 99% (e.g., at least 90%) identical to that of SEQ ID NO: 70; or
d) an HC and an LC comprising the amino acid sequences of SEQ ID NOs: 3 and 70, respectively.

In certain embodiments, the immunoconjugate of the invention comprises an anti-ROR1 antibody, or an antigen-binding fragment thereof, wherein the antibody comprises:
a) a heavy chain variable domain or region (V_{H}) comprising an amino acid sequence at least 80%, 85%, 90%, 91%, 92%, 93%, 94%, 95%, 96%, 97%, 98%, or 99% (e.g., at least 90%) identical to that of SEQ ID NO: 67, and a light chain variable domain or region (V_{L}) comprising an amino acid sequence at least 80%, 85%, 90%, 91%, 92%, 93%, 94%, 95%, 96%, 97%, 98%, or 99% (e.g., at least 90%) identical to that of SEQ ID NO: 68;
b) a V_{H} and a V_{L} comprising the amino acid sequences of SEQ ID NOs: 67 and 68, respectively;
c) a heavy chain (HC) comprising an amino acid sequence at least 80%, 85%, 90%, 91%, 92%, 93%, 94%, 95%, 96%, 97%, 98%, or 99% (e.g., at least 90%) identical to that of SEQ ID NO: 69 and a light chain (LC) comprising an amino acid sequence 80%, 85%, 90%, 91%, 92%, 93%, 94%, 95%, 96%, 97%, 98%, or 99% (e.g., at least 90%) identical to that of SEQ ID NO: 70; or
d) an HC and an LC comprising the amino acid sequences of SEQ ID NOs: 69 and 70, respectively.

In certain embodiments, the V_{H} and V_{L} of the antibody respectively comprise the amino acid sequences of:
a) SEQ ID NOs: 5 and 50;
b) SEQ ID NOs: 48 and 6;
c) SEQ ID NOs: 48 and 50;
d) SEQ ID NOs: 67 and 50; or
e) SEQ ID NOs: 48 and 68.

In some embodiments, the antibody or fragment comprises a human IgG₁, IgG₂, IgG₃, or IgG₄ constant region and optionally a human κ constant region.

In certain embodiments, the HC and LC of the antibody respectively comprise the amino acid sequences of:
a) SEQ ID NOs: 3 and 49;
b) SEQ ID NOs: 47 and 4;
c) SEQ ID NOs: 47 and 49;
d) SEQ ID NOs: 69 and 49; or
3) SEQ ID NOs: 47 and 70.

In certain embodiments, the immunoconjugate of the invention comprises an antibody or fragment thereof derived from a murine antibody with the V_{H} and V_{L} amino acid sequences of (i) SEQ ID NOs: 25 and 26, respectively; (ii) SEQ ID NOs: 35 and 36, respectively; or (iii) SEQ ID NOs: 45 and 46, respectively. Antibodies derived from these sequences may be, e.g., antibodies that have been humanized or joined to a human Fc region (e.g., chimeric). For example, the antibody or an antigen-binding fragment in the immunoconjugate comprises:
a) a V_{H} comprising an amino acid sequence at least 80%, 85%, 90%, 91%, 92%, 93%, 94%, 95%, 96%, 97%, 98%, or 99% identical to that of SEQ ID NO: 45 and a V_{L} comprising an amino acid sequence at least 80%, 85%, 90%, 91%, 92%, 93%, 94%, 95%, 96%, 97%, 98%, or 99% identical to that of SEQ ID NO: 46;
b) a V_{H} comprising the amino acid sequence of SEQ ID NO: 45 and a V_{L} comprising the amino acid sequence of SEQ ID NO: 46;
c) a V_{H} comprising an amino acid sequence at least 80%, 85%, 90%, 91%, 92%, 93%, 94%, 95%, 96%, 97%, 98%, or 99% identical to that of SEQ ID NO: 25 and a V_{L} comprising an amino acid sequence at least 80%, 85%, 90%, 91%, 92%, 93%, 94%, 95%, 96%, 97%, 98%, or 99% identical to that of SEQ ID NO: 26; or
d) a V_{H} comprising the amino acid sequence of SEQ ID NO: 25 and a V_{L} comprising the amino acid sequence of SEQ ID NO: 26.

Exemplary coding sequences for the aforementioned antibodies are shown in Table 6 below. For example, the antibody in the immunoconjugate may comprise:
a) a V_{H} encoded by (i) nucleotides 73-420 of SEQ ID NO: 21, or (ii) SEQ ID NO: 23; and a V_{L} encoded by SEQ ID NO: 22 or 24;
b) a V_{H} encoded by SEQ ID NO: 52 and a V_{L} encoded by SEQ ID NO: 54;
c) a V_{H} encoded by SEQ ID NO: 33 and a V_{L} encoded by SEQ ID NO: 34;
d) an HC encoded by nucleotides 73-1,410 of SEQ ID NO: 19 and an LC encoded by nucleotides 73-714 of SEQ ID NO: 20; or
e) an HC encoded by SEQ ID NO: 51 and an LC encoded by nucleotides SEQ ID NO: 53.

In certain embodiments, the immunoconjugate of the invention comprises an antigen-binding fragment of an anti-ROR1 antibody, wherein the antigen-binding fragment comprises the sequence of any one of SEQ ID NOs: 60-64. In certain embodiments, the antigen-binding fragment comprises the V_{H} and V_{L} amino acid sequences of:
a) SEQ ID NOs: 5 and 6;
b) SEQ ID NOs: 5 and 50;
c) SEQ ID NOs: 48 and 6;
d) SEQ ID NOs: 48 and 50;
e) SEQ ID NOs: 45 and 46;
f) SEQ ID NOs: 25 and 26;
g) SEQ ID NOs: 67 and 6;
h) SEQ ID NOs: 5 and 68; or
i) SEQ ID NOs: 67 and 68,
   wherein the V_{H} amino acid sequence is optionally linked to the amino acid sequence of SEQ ID NO: 58, and/or the V_{L} amino acid sequence is optionally linked to the amino acid sequence of SEQ ID NO: 59.

In some embodiments, an antibody or antigen-binding fragment described herein may comprise one or more Fc region mutations. For example, where the antibody is of the IgG1 subclass, any combination of the amino acid residues at positions 234, 235, and 329 may be mutated, for example from Leu to Ala (L234A, L235A) and/or from Pro to Ala or Gly (P329A or P329G). These mutations reduce effector function of the Fc region of IgG1 antibodies. For example, the antibody may have the “LALA” mutations (L234A/L235A) in the Fc region, thus hindering the antibody's binding to human FcγR (Fc gamma receptors). Antibodies with such “effectorless” mutations are advantageous because they have a low level of secondary effector functions and do not deplete effector T cells or target other non-malignant cells. Consequently, such antibodies may display less non-specific toxicity and broaden the therapeutic window.

3. Antibody Sequence Comparison

Percent (%) sequence identity with respect to a reference polypeptide sequence refers to the percentage of amino acid residues in a candidate sequence that are identical with the amino acid residues in the reference sequence, after aligning the sequences and introducing gaps, if necessary, to achieve the maximum percent sequence identity. Alignment for purposes of determining percent amino acid sequence identity can be achieved in various ways that are known; for instance, using publicly available computer software such as BLAST, BLAST-2, ALIGN, ALIGN-2, or Megalign (DNASTAR). For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparison, the % amino acid sequence identity of a given amino acid sequence A to a given amino acid sequence B is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In some embodiments, amino acid sequence variants of the antibodies provided herein are contemplated. A variant typically differs from a polypeptide specifically disclosed herein in one or more substitutions, deletions, additions and/or insertions. Such variants can be naturally occurring or can be synthetically generated, for example, by modifying one or more of the above polypeptide sequences of the invention and evaluating one or more biological activities of the polypeptide as described herein and/or using any of a number of known techniques. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

As used herein, the term “substantially identical” refers to two or more sequences having a percentage of sequential units (e.g., amino acid residues) which are the same when compared and aligned for maximum correspondence over a comparison window, or a designated region as measured using comparison algorithms. By way of example, two or more sequences may be “substantially identical” if the sequential units are about 60% identical, about 65% identical, about 70% identical, about 75% identical, about 80% identical, about 85% identical, about 90% identical, about 95% identical, about 96% identical, about 97% identical, about 98% identical, or about 99% identical over a specified region. Such percentages describe the “percent identity” between two sequences.

4. Making and Modification of ROR1 Antibodies

Anti-ROR1 antibodies for use in the immunoconjugates of the present invention can be made by immunizing an animal with human ROR1 or a fragment of human ROR1 protein. Antibodies that bind to the immunizing fragment with high affinity (e.g., with a K_{D} in the nM or lower range) can be screened by using routine methods such as ELISA.

If the antibody is a non-human antibody, it can be humanized. A “humanized” antibody is an antibody in which all or substantially all CDR amino acid residues are derived from a non-human (e.g., mouse or rat) antibody and all or substantially all FR amino acid residues are derived from human FRs. A humanized antibody optionally may include at least a portion of a constant region derived from a human antibody. A “humanized form” of a non-human antibody refers to a variant of the non-human antibody that has undergone humanization, typically to reduce immunogenicity to humans, while retaining the antigen-binding specificity and affinity of the parental non-human antibody. In some embodiments, some FR residues in a humanized antibody are substituted with corresponding residues from the cognate non-human antibody to restore or improve the resultant antibody's antigen-binding specificity and/or affinity.

ROR1 antibodies or fragments may be manufactured recombinantly in mammalian host cells containing coding sequences for the ROR1 antibodies or fragments, wherein the coding sequences are operably linked to transcription-regulatory elements suitable for expression in the host cells. The coding sequences may be introduced into the host cells on one or more vectors. Useful mammalian host cells include, inter alia, Chinese hamster ovary (CHO) cells, NS0 cells, SP2 cells, HEK-293T cells, 293 Freestyle cells (Invitrogen), NIH-3T3 cells, HeLa cells, baby hamster kidney (BHK) cells, African green monkey kidney cells (COS), human hepatocellular carcinoma cells (e.g., Hep G2), and A549 cells. Cell lines may be selected based on their expression levels. Other cell lines that may be used include insect cell lines, such as Sf9 or Sf21 cells, and yeast cell lines.

In some embodiments, a parent ROR1 antibody may be engineered by introducing one or more amino acid substitutions to improve the antibody's antigen binding, to decrease immunogenicity (e.g., de-immunize; see, e.g., Jones et al., Methods Mol Biol. (2009) 525:405-23), and/or to improve antibody-dependent cell-mediated cytotoxicity (ADCC) or complement-dependent cytotoxicity (CDC).

In some embodiments, substitutions, insertions, or deletions may be made within one or more CDRs, wherein the mutations do not substantially reduce the antibody's binding to its antigen. For example, conservative substitutions that do not substantially reduce binding affinity may be made.

Alterations (e.g., substitutions) may be made in CDRs to improve antibody affinity. CDR residues involved in antigen binding may be identified by using, e.g., alanine scanning mutagenesis or computer modeling. HCDR3 and LCDR3 in particular are often targeted. A crystal structure of an antigen-antibody complex may also be used to identify contact points between the antibody and its antigen. Such contact residues and their neighboring residues may be targeted for mutations. Variants may be screened to determine whether they obtain the desired properties. In vitro affinity maturation (e.g., using error-prone PCR, chain shuffling, randomization of CDRs, or oligonucleotide-directed mutagenesis) may also be used to improve antibody affinity (see, e.g., Hoogenboom et al., Methods in Molecular Biology (2001) 178:1-37).

Amino acid sequence insertions and deletions made to an antibody or antibody fragment include amino- and/or carboxyl-terminal fusions ranging in length from one or a few residues to polypeptides containing a hundred or more residues, as well as intra-sequence insertions and deletions of single or multiple amino acid residues. Examples of terminal insertions include an antibody with an N-terminal methionyl residue. Other insertional variants of the antibody molecule include the fusion of the N- or C-terminus of the antibody to an enzyme (e.g., for ADEPT) or a polypeptide that increases the serum half-life of the antibody. Examples of intra-sequence insertion variants of the antibody molecules include an insertion of 3 amino acids in the light chain. Examples of terminal deletions include an antibody with a deletion of 7 or fewer amino acids at an end of the light chain, and the removal of the C-terminal lysine in the heavy chain.

In some embodiments, the ROR1 antibodies are altered to increase or decrease their glycosylation (e.g., by altering the amino acid sequence such that one or more glycosylation sites are created or removed). A carbohydrate attached to an Fc region of an antibody may be altered. Native antibodies from mammalian cells typically comprise a branched, biantennary oligosaccharide attached by an N-linkage to Asn₂₉₇ of the CH₂ domain of the Fc region (see, e.g., Wright et al., TIBTECH (1997) 15:26-32). Asn₂₉₇ refers to the asparagine residue located at about position 297 in the Fc region (EU numbering of Fc region residues; see, e.g., Edelman et al., PNAS (1969) 63(1):78-85). However, Asn₂₉₇ may also be located about ±3 amino acids upstream or downstream of position 297, i.e., between positions 294 and 300, due to minor sequence variations in antibodies. The oligosaccharide can be any of various carbohydrates, e.g., mannose, N-acetyl glucosamine (GlcNAc), galactose, sialic acid, or fucose attached to a GlcNAc in the stem of the biantennar oligosaccharide structure. Modifications of the oligosaccharide in an antibody can be made, for example, to create antibody variants with certain improved properties. Antibody glycosylation variants can have improved ADCC and/or CDC function.

In some embodiments, antibody variants are provided having a carbohydrate structure that has no or a reduced level of fucose attached (directly or indirectly) to an Fc region. For example, the amount of fucose in such an antibody may be from 1% to 80%, from 1% to 65%, from 5% to 65% or from 20% to 40%. The amount of fucose is determined by calculating the average amount of fucose within the sugar chain at Asn₂₉₇, relative to the sum of all glycostructures attached to Asn₂₉₇ (see, e.g., PCT Patent Publication WO 2008/077546). Such fucosylation variants can have improved ADCC function (see, e.g., Okazaki et al., J Mol Biol. (2004) 336:1239-49; and Yamane-Ohnuki et al., Biotech Bioeng. (2004) 87:614). Cell lines (e.g., knockout cell lines) can be used to produce defucosylated antibodies, e.g., Lec13 CHO cells deficient in protein fucosylation and alpha-1,6-fucosyltransferase gene (FUT8) knockout CHO cells (see, e.g., Ripka et al., Arch Biochem Biophys. (1986) 249:533-45; Yamane-Ohnuki et al., Biotech Bioeng. (2004) 87:614; and Kanda et al., Biotechnol Bioeng. 94(4): (2006) 680-8). Other antibody glycosylation variants as described in, e.g., U.S. Pat. No. 6,602,684, may also be made to the ROR1 antibodies or antibody fragments for use in the present immunoconjugates.

In some embodiments, one or more amino acid modifications may be introduced into the Fc region of a ROR1 antibody to generate a ROR1 antibody with a variant Fc region that confers new properties to the antibody. A variant Fc region may comprise a human Fc region sequence (e.g., a human IgG₁, IgG₂, IgG₃ or IgG₄ Fc region) comprising an amino acid modification (e.g., a substitution) at one or more amino acid positions. For example, a ROR1 antibody with a variant Fc region may possess some but not all effector functions, which makes it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. Non-limiting examples of in vitro assays to assess ADCC activity of a molecule of interest are described in U.S. Pat. Nos. 5,500,362 and 5,821,337. Alternatively, non-radioactive assay methods may be employed (e.g., ACTI™ and CytoTox 96® non-radioactive cytotoxicity assays). Useful effector cells for such assays include peripheral blood mononuclear cells (PBMCs), monocytes, macrophages, and natural killer (NK) cells.

Antibodies can have increased half-lives and improved binding to the neonatal Fc receptor (FcRn) (see, e.g., U.S. Patent Publication 2005/0014934). Such antibodies can comprise an Fc region with one or more substitutions therein which improve binding of the Fc region to FcRn, and include those with substitutions at one or more of Fc region residues: 238, 256, 265, 272, 286, 303, 305, 307, 311, 312, 317, 340, 356, 360, 362, 376, 378, 380, 382, 413, 424 and 434 according to the EU numbering system (see, e.g., U.S. Pat. No. 7,371,826). Other examples of Fc region variants are also contemplated (see, e.g., Duncan & Winter, Nature (1988) 322:738-40; U.S. Pat. Nos. 5,648,260 and 5,624,821; and PCT Publication WO 94/29351).

In some embodiments, it may be desirable to create cysteine engineered antibodies, e.g., “thioMAbs,” in which one or more residues of an antibody are substituted with cysteine residues. In some embodiments, the substituted residues occur at accessible sites of the antibody. Reactive thiol groups can be positioned at sites for conjugation to other moieties, such as drug moieties or linker drug moieties, to create an immunoconjugate. Any one or more of the following residues may be substituted with cysteine: V205 (Kabat numbering) of the light chain; A118 (EU numbering) of the heavy chain; and S400 (EU numbering) of the heavy chain Fc region.

An antibody provided herein may be further modified to include non-proteinaceous moieties. The moieties suitable for derivatization of the antibody include but are not limited to water soluble polymers. The term “polymer,” as used herein, refers to a molecule composed of repeated subunits; such molecules include, but are not limited to, polypeptides, polynucleotides, or polysaccharides, or polyalkylene glycols. Non-limiting examples of water soluble polymers are polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyamino acids (either homopolymers or random copolymers), and dextran or poly(N-vinyl pyrrolidone)-polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if two or more polymers are attached, they can be the same or different molecules.

5. Cytotoxic Drug Moieties

An immunoconjugate of the invention comprises an anti-ROR1 antibody or an antigen-binding fragment thereof conjugated to exatecan (DX8951) or an analog (e.g., a derivative) thereof, wherein exatecan has the following structural formula (I):

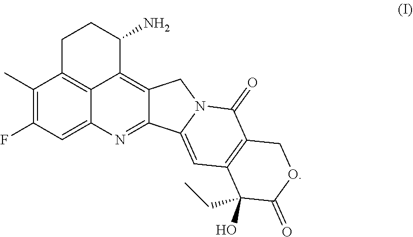 [SMILES: CC[C@@]1(O)C(=O)OCc2c1cc1n(c2=O)Cc2c-1nc1cc(F)c(C)c3c1c2[C@@H](N)CC3]

The antibody may attach to the compound of formula I at the amino group or at the hydroxyl group through a linker (e.g., cleavable or noncleavable linker).

In some embodiments, the antibody is attached to an exatecan analog having the following structure formula (II):

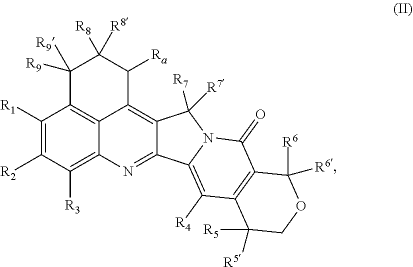

wherein each of R₁, R₃, R₄, R₅, R₅′, R₆, R₆′, R₇, R₇′, R₈, R₈′, R₉, and R₉′, independently, is H, F, Cl, Br, I, —ORₐ, or a substituted or unsubstituted C₁-C₆ alkyl group; R₂ is F, Cl, Br, or I; and Rₐ is independently selected from H, deuterium, halo, amino, hydroxy, thiol, cyano, formyl, alkyl, haloalkyl, alkenyl, haloalkenyl, alkynyl, haloalkynyl, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio.

“Amino” refers to unsubstituted amino and substituted amino groups, for example, primary amines, secondary amines, tertiary amines and quaternary amines. Specifically, “amino” refers to —(CH₂)ₓNR_{b}R_{c}, wherein R_{b} and R_{c}, both directly connected to the N, can be independently selected from H, deuterium, halo, hydroxy, cyano, formyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, a nitrogen protective group, —(CO)-alkyl, —(CO)—O-alkyl, or —S(O)ₙR_{d} (n=0 to 2, R_{d} is directly connected to S), wherein R_{d} is independently selected from H, deuterium, halo, amino, hydroxy, thiol, cyano, formyl, alkyl, haloalkyl, alkenyl, haloalkenyl, alkynyl, haloalkynyl, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio, and x=0 to 10.

An alkyl, alkenyl, alkynyl, alkoxy, or alkanoyl group may be further substituted by deuterium, halo, hydroxy, cyano, formyl, nitro, amino, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, —(CO)-alkyl, —(CO)—O-alkyl, or —S(O)ₙRₐ (n=0 to 2, R_{d} is directly connected to S), wherein R_{d} is independently selected from H, deuterium, halo, amino, hydroxy, thiol, cyano, formyl, alkyl, haloalkyl, alkenyl, haloalkenyl, alkynyl, haloalkynyl, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio, and x=0 to 10.

In preferred embodiments, each of R₃, R₄, R₆, R₆′, R₇, R₇′, R₈, R₈′, R₉, and R₉′, independently, is H, R₁ is substituted or unsubstituted C₁-C₂ alkyl group; each of R₅ and R₅′, independently, is —OH or a substituted or unsubstituted C₁-C₂ alkyl group; R₂ is F; and Ra is amino, hydroxy, substituted or unsubstituted C₁-C₂ alkyl group, or substituted or unsubstituted alkanoyl group. In still preferred embodiments, Ra is —NR_{b}R_{c}, wherein R_{b} and R_{c}, independently, is H, hydroxy, substituted or unsubstituted C₁-C₂ alkyl group, or substituted or unsubstituted alkanoyl group. An exatecan analog has substantially similar biological (e.g., cytotoxic) profiles as exatecan. The antibody may attach to the compound of formula II at Ra, or at R₅ or R₅′ through a linker (e.g., cleavable or noncleavable linker).

In some embodiments, the ROR1 immunoconjugate is conjugated to an exatecan analog of structural formula (III):

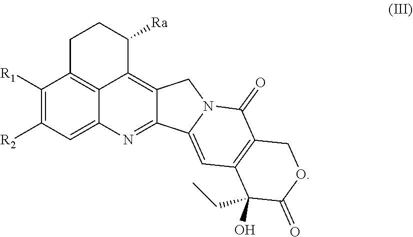

wherein R₁ is H or a substituted or unsubstituted C₁-C₆ alkyl group, R₂ is F, Cl, Br, or I, and Ra is amino, hydroxy, substituted or unsubstituted C₁-C₂ alkyl group, or substituted or unsubstituted alkanoyl group. In preferred embodiments, Ra is —NR_{b}R_{c}, wherein R_{b} and R_{c}, independently, is H, hydroxy, substituted or unsubstituted C₁-C₂ alkyl group, or substituted or unsubstituted alkanoyl group. The antibody may attach to the compound of formula III at the amino group or at the hydroxyl group through a linker (e.g., cleavable or noncleavable linker).

In some embodiments, the ROR1 immunoconjugate is conjugated to an exatecan derivative of structural formula (IV) or (V):

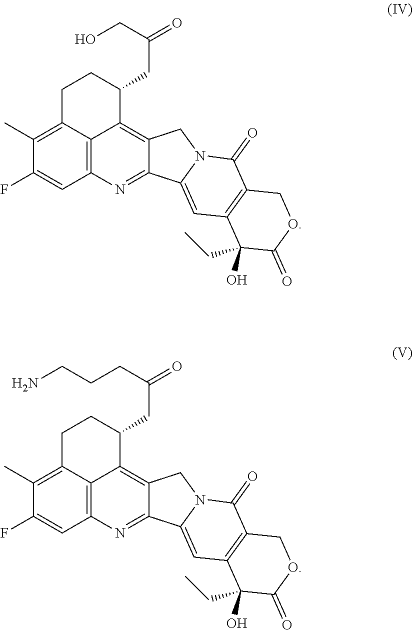 [SMILES: CC[C@@]1(O)C(=O)OCc2c1cc1n(c2=O)Cc2c-1nc1cc(F)c(C)c3c1c2[C@@H](CC(=O)CO)CC3 ; CC[C@@]1(O)C(=O)OCc2c1cc1n(c2=O)Cc2c-1nc1cc(F)c(C)c3c1c2[C@@H](CC(=O)CCCN)CC3]

In some embodiments, the ROR1 immunoconjugate is conjugated to an exatecan derivative of structural formula (VI), (VII), (VIII), (IX), (X), or (XI):

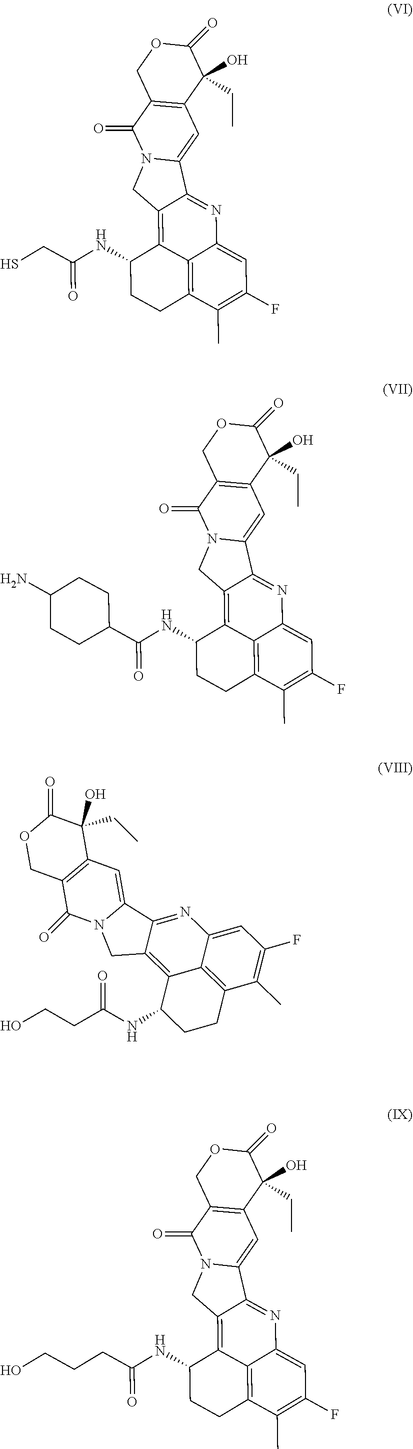 [SMILES: CC[C@@]1(O)C(=O)OCc2c1cc1n(c2=O)Cc2c-1nc1cc(F)c(C)c3c1c2[C@@H](NC(=O)CS)CC3 ; CC[C@@]1(O)C(=O)OCc2c1cc1n(c2=O)Cc2c-1nc1cc(F)c(C)c3c1c2[C@@H](NC(=O)C1CCC(N)CC1)CC3 ; CC[C@@]1(O)C(=O)OCc2c1cc1n(c2=O)Cc2c-1nc1cc(F)c(C)c3c1c2[C@@H](NC(=O)CCO)CC3 ; CC[C@@]1(O)C(=O)OCc2c1cc1n(c2=O)Cc2c-1nc1cc(F)c(C)c3c1c2[C@@H](NC(=O)CCCO)CC3]

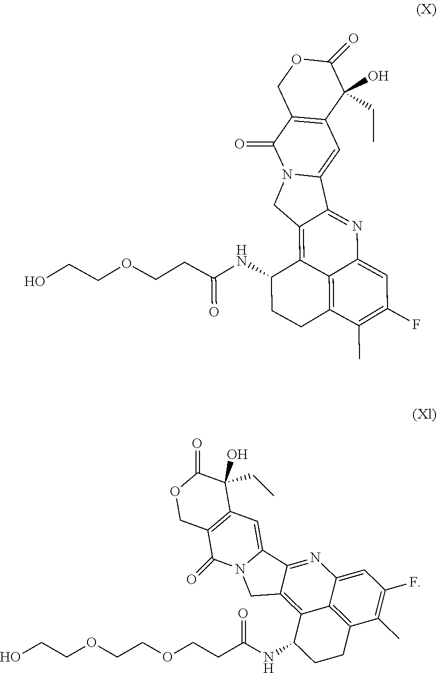 [SMILES: CC[C@@]1(O)C(=O)OCc2c1cc1n(c2=O)Cc2c-1nc1cc(F)c(C)c3c1c2[C@@H](NC(=O)CCOCCO)CC3 ; CC[C@@]1(O)C(=O)OCc2c1cc1n(c2=O)Cc2c-1nc1cc(F)c(C)c3c1c2[C@@H](NC(=O)CCOCCOCCO)CC3]

In some embodiments, once metabolized inside a tumor cell, the immunoconjugate may release a compound comprising formula I, II, III, IV, V, VI, VII, VIII, IX, X, or XI.

As described herein, the chemical structure of exatecan or its analog or derivative shown above is prior to its chemical conjugation to the Ab or linker. It will be apparent to the skilled person in the ADC art whether a certain chemical entity disclosed herein is a precursor molecule or the D (payload) component in the final immunoconjugate product.

In some embodiments, the average number of the drug moiety to the antibody in the immunoconjugate (i.e., drug-to-antibody ratio or DAR) is 1; or at least 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 6, 7, 8, 9, or 10. Exemplary methods for measuring DAR are described in the Examples below.

6. Linkers

In certain embodiments of an immunoconjugate of the invention, the anti-ROR1 antibody can be conjugated directly to exatecan or a derivative thereof, or can be conjugated via a linker. Suitable linkers include, for example, cleavable and non-cleavable linkers. In some embodiments, the linker is a cleavable linker. A cleavable linker refers to a linker that comprises a cleavable moiety and is typically susceptible to cleavage under intracellular conditions. Suitable cleavable linkers include, for example, peptide linkers cleavable by an intracellular protease (such as a lysosomal protease or an endosomal protease), and acid-cleavable linkers. In certain embodiments, the linker can be a dipeptide, such as a valine-citrulline (Val-Cit or VCit), valine-alanine (Val-Ala or VA), or a phenylalanine-lysine (Phe-Lys or FK) linker. In certain embodiments, the linker can be a tripeptide, such as a glutamic acid-valine-citrulline (Glu-Val-Cit or EVCit) linker. In certain embodiments, the linker can be a tetrapeptide, such as a glycine-glycine-phenylalanine-glycine (GGFG) linker. In yet other embodiments, the linker can be a polypeptide having four or more amino acid residues, wherein one or more of these amino acid residues contain lipophilic side chains. Other suitable linkers include linkers hydrolyzable at a pH of less than 5.5, such as a hydrazone linker. Additional suitable cleavable linkers include disulfide linkers. In some embodiments, the linker is a non-polymeric linker. In some cases, the linker is a non-peptide linker or a linker that does not contain an amino acid residue.

In some embodiments, the linker includes a C₁-C₆ alkyl group (e.g., a C₅, C₄, C₃, C₂, or C₁ alkyl group). As used herein the term “alkyl” refers to a straight or branched hydrocarbon chain radical consisting solely of carbon and hydrogen atoms, containing no unsaturation. C₁-Cₓ includes C₁-C₂, C₁-C₃, . . . , C₁-Cₓ, where x is an integer. C₁-Cₓ refers to the number of carbon atoms in the designated group. In some embodiments, an alkyl comprises one to eight carbon atoms (C₁-C₈ alkyl). In some embodiments, an alkyl comprises two to six carbon atoms (C₂-C₆ alkyl).

As used herein, a linker prior to the chemical reaction to link the Ab (antibody or fragment) and D (payload) components of the immunoconjugate is also called a “linker precursor.” It will be apparent to the skilled person in the ADC art whether a certain chemical entity disclosed herein is a linker precursor based on its reactive capabilities, or a linker component in the final immunoconjugate product.

In some embodiments, the linkage between the Ab and D components of the immunoconjugate may be formed through reaction of the components with a linker having a reactive functional group that may comprise, e.g., a nucleophilic group that is reactive to an electrophilic group present on a binding moiety. Exemplary electrophilic groups include carbonyl groups such as aldehydes, ketones, carboxylic acids, esters, amides, enones, acyl halides, and acid anhydrides. In particular embodiments, the reactive functional group is an aldehyde. Exemplary nucleophilic groups include hydrazide, oxime, amino, hydrazine, thiosemicarbazone, hydrazine carboxylate, and arylhydrazide.

In some embodiments, the conjugation of the linker/payload to the antibody or fragment may be formed through reaction with a maleimide group (which may also be referred to as a maleimide spacer). In certain embodiments, the maleimide group is maleimidocaproyl (mc); thus, the linker/payload is conjugated to the antibody or fragment through reaction between a residue on the antibody or fragment and the me group in the linker precursor. In some embodiments, the maleimide group comprises a maleimidomethyl group, such as succinimidyl-4-(N-maleimidomethyl)cyclohexane-1-carboxylate (SMCC) or sulfosuccinimidyl-4-(N-maleimidomethyl)cyclohexane-1-carboxylate (sulfo-SMCC), as described herein.

In some embodiments, the maleimide group is a maleimidoaryl group, such as a maleimidophenyl group. In some embodiments, the phenyl ring of the maleimidophenyl group is substituted. In some embodiments, the phenyl ring is substituted by one or more electron withdrawing groups. In certain embodiments, the electron withdrawing group is ortho or para to the maleimide group. Examples of electron withdrawing groups include, but are not limited to, F, Cl, Br, CO₂H, SO₃H, NO₂, CF₃, and CN. In some embodiments, the phenyl ring is substituted by one or more amino groups. In certain embodiments, the amino group is ortho to the maleimide group. In certain embodiments, the amino group is —CH₂NHR_{b}, wherein R_{b} is H or a C₁-C₈ alkyl group. In certain embodiments, R_{b} is H, Me, or isopropyl. In some embodiments, the phenyl ring is substituted by one or more PEG chains. In certain embodiments, the PEG chain is ortho to the maleimide group. In some embodiments, the phenyl ring is substituted by one or more glucuronide moieties. In certain embodiments, the glucuronide moiety is ortho to the maleimide group. In some embodiments, the phenyl ring is substituted by one or more galactose moieties or derivatives thereof. In certain embodiments, the galactose moiety or a derivative thereof is ortho to the maleimide group.

In some embodiments, the maleimide group comprises a heterocyclic group, such as a 3-8 membered heterocyclic ring. In some embodiments, the heterocyclic group contains one or more heteroatoms selected from N, O, and S. In certain embodiments, the heterocyclic group is a dioxolane ring.

In some embodiments, the maleimide group is a self-stabilizing maleimide. In some embodiments, the self-stabilizing maleimide utilizes diaminopropionic acid (DPR) to incorporate a basic amino group adjacent to the maleimide to provide intramolecular catalysis of thiosuccinimide ring hydrolysis, thereby decreasing the ability of the maleimide to undergo an elimination reaction through a retro-Michael reaction. In some embodiments, the self-stabilizing maleimide is a maleimide group described in Lyon et al., Nat Biotechnol. (2014) 32(10):1059-62. In certain embodiments, the linker precursor comprises a self-stabilizing maleimide. In certain embodiments, the linker precursor is a self-stabilizing maleimide.

In some embodiments, the linker may include a peptide moiety. In some embodiments, the peptide moiety comprises at least 2, 3, 4, 5, 6, 7, 8, or more amino acid residues, wherein one or more of these amino acid residues contain lipophilic side chains. In some embodiments, the peptide moiety is cleavable (e.g., either enzymatically or chemically). In some embodiments, the peptide moiety is non-cleavable. In some embodiments, the peptide moiety comprises Val-Cit (valine-citrulline), Val-Ala (valine-alanine), Phe-Lys (phenylalanine-lysine), Val-Lys (valine-lysine), Ala-Lys (alanine-lysine), Val-Arg (valine-arginine), Phe-Cit (phenylalanine-citrulline), Phe-Arg (phenylalanine-arginine), Leu-Cit (leucine-citrulline), Ile-Cit (isoleucine-citrulline), Trp-Cit (tryptophan-citrulline), Phe-Ala (phenylalanine-alanine), Glu-Val-Cit (glutamic acid-valine-citrulline), Gly-Phe-Lys (glycine-phenylalanine-lysine), Phe-Phe-Lys (phenylalanine-phenylalanine-lysine), Gly-Gly-Phe-Gly (SEQ ID NO: 55), Ala-Leu-Ala-Leu (SEQ ID NO: 56), or Gly-Phe-Leu-Gly (SEQ ID NO: 57). In certain embodiments, the linker comprises Val-Cit (VCit). In certain embodiments, the linker comprises tetrapeptide GGFG.

In some embodiments, the linker may include a benzoic acid or benzyloxy group, or a derivative thereof. For example, the linker may comprise para-amino-benzoic acid (PABA). In some embodiments, the linker includes a para-amino-benzyloxycarbonyl (PAB) group. In some embodiments, the linker comprises gamma-amino-butyric acid (GABA).

In some embodiments, the linkage between the Ab and D components of the immunoconjugate may be formed through reaction of the components with a linker comprising a maleimide group, a peptide moiety, and/or a benzoic acid (e.g., PABA) or benzyloxycarbonyl (PAB) group, in any combination. In certain embodiments, the maleimide group is maleimidocaproyl (mc). In certain embodiments, the peptide group is Val-Cit (VCit). In certain embodiments, the linker comprises a Val-Cit-PAB or Val-Cit-PABA group. In certain embodiments, the conjugation of the linker to the antibody or fragment may be formed from an mc-Val-Cit-PABA group. In certain embodiments, the conjugation of the linker to the antibody or fragment may be formed from an mc-Val-Cit group. In certain embodiments, the linkage between the antibody or fragment and the drug moiety may be formed from an mc-Val-Cit-PAB group. In certain embodiments, the linkage between the antibody or fragment and the drug moiety may be formed from a mal-Val-Cit-PAB group.

In some embodiments, the conjugation of the linker/payload to the antibody or fragment may be formed through reaction with a quaternary vinyl pyridinyl group. Thus, the linker/payload is conjugated to the antibody or fragment through reaction between a residue on the antibody or fragment and the vinyl group in the linker precursor.

Suitable linkers for use in the immunoconjugates of the invention may include, e.g., linkers that are intracellularly cleavable with high extracellular stability. In certain embodiments, the linker comprises a functional group that allows for attachment of the linker to any of the antibodies or fragments described herein (e.g., a maleimide derivative). In certain embodiments, the linker (or precursor) comprises 6-maleimidocaproyl (MC), maleimidopropanoyl (MP), valine-citrulline (VCit), alanine-phenylalanine (AP), p-aminobenzyloxycarbonyl (PAB), N-succinimidyl 4-(2-pyridylthio) pentanoate (SPP), N-succinimidyl 4-(N-maleimidomethyl) cyclohexane-1-carboxylate (SMCC), N-succinimidyl (4-iodo-acetyl) aminobenzoate (SIAB), 6-maleimidocaproyl-valine-citrulline (MC-VC), 6-maleimidocaproyl-valine-citrulline-p-aminobenzyloxycarbonyl (MC-VC-PAB), N-succinimidyl-1-carboxylate-valine-citrulline-p-aminobenzyloxycarbonyl (SC-VC-PAB), 6-maleimidocaproyl-polyethylene glycol-valine-citrulline (MC-PEG4-VC), 6-maleimidocaproyl-polyethylene glycol-valine-alanine (MC-PEG4-VA), or MC-PEG8-VC-PAB. In some embodiments, the linker prior to conjugation reaction (aka linker precursor) is 6-maleimidocaproyl-valine-citrulline-p-aminobenzyloxycarbonyl (MC-VC-PAB), or N-succinimidyl-1-carboxylate-valine-citrulline-p-aminobenzyloxycarbonyl (SC-VC-PAB). In some embodiments, the linker is a 6-maleimidocaproyl (MC) linker. In some embodiments, the linker is a maleimidopropanoyl (MP) linker. In some embodiments, the linker is a valine-citrulline (VC) linker. In some embodiments, the linker is an alanine-phenylalanine (AP) linker. In some embodiments, the linker is a p-aminobenzyloxycarbonyl (PAB) linker. In some embodiments, the linker is an N-succinimidyl 4-(2-pyridylthio) pentanoate (SPP) linker. In some embodiments, the linker is an N-succinimidyl 4-(N-maleimidomethyl) cyclohexane-1-carboxylate (SMCC) linker. In some embodiments, the linker is an N-succinimidyl (4-iodo-acetyl) aminobenzoate (SIAB) linker. In some embodiments, the linker is a 6-maleimidocaproyl-valine-citrulline (MC-VC) linker. In some embodiments, the linker is a 6-maleimidocaproyl-valine-citrulline-p-aminobenzyloxycarbonyl (MC-VC-PAB) linker. In some embodiments, the linker is an N-succinimidyl-1-carboxylate-valine-citrulline-p-aminobenzyloxycarbonyl (SC-VC-PAB) linker. In some embodiments, the linker further comprises a spacer between the linker and the cytotoxic moiety. In some embodiments, the spacer is a heteroatom. In some embodiments, the spacer is an alkyl chain. In some embodiments, the spacer is an alkyl chain comprising one or more heteroatoms. In some embodiments, the spacer is a carbonyl group. In some embodiments, the linker is a homobifunctional linker or a heterobifunctional linker.

In some embodiments, the linkers described herein may be attached to the antibodies or antigen-binding fragments described herein at a naturally occurring amino acid residue such as a lysine or a reduced cysteine. In some embodiments, the linkers can be attached to a non-natural amino acid (e.g., azidophenylalanine, p-acetylphenylalanine, or p-azidomethylphenylalanine) by way of an alkyne/azide “click” reaction, carbonyl condensations, Michael-type additions, and Mizoroki-Heck substitutions. Additional linker sites can be added genetically and may comprise a polypeptide motif that allows enzymatic addition of the linker. Such polypeptide motifs can comprise, e.g., a glutamine tag (e.g., LLQGA), an aldehyde tag (e.g., CxPxR, where x is any amino acid), a sortase motif (e.g., LPxTG, where x is any amino acid or NPQTN), or a BirA tag (e.g., GFEIDKVWYDLDA). Attaching linkers to a glutamine tag may be achieved by using a bacterial transglutaminase. Attaching linkers to an aldehyde tag is achieved by using formylglycine-generating enzyme (FGE), which oxidizes the cysteine residue of the consensus sequence, creating an aldehyde; this aldehyde can be reacted with an aminooxy group on a linker to form a stable oxime. Attaching linkers to a sortase motif can be achieved by using a bacterial transpeptidase that recognizes a C-terminal LPxTG sequence or NPQTN sequence, cleaves the TG or TN bond, and facilitates—via a thioacyl enzyme-threonine intermediate—the nucleophilic attack of the incoming protein alpha amine on the threonine. The attacking residue can be a glycine dimer or trimer of a linker or added to a linker. Attaching linkers to a sortase motif or a BirA tag can be achieved by using a biotin ligase. In certain embodiments, the linker is not attached at a lysine residue. In certain embodiments, the linker is only attached to a cysteine residue. In certain embodiments, the cysteine residue has been engineered into the antibody by converting one or more non-cysteine residues on the light chain and/or heavy chain to a cysteine. In certain embodiments, cysteine conversion does not interfere with antigen binding. In certain embodiments, selenocysteine is incorporated into the antibody by genetic modification. See, e.g., Sochaj et al., Biotechnology Advances (2015) 33:775-84.

In some embodiments, the linker is conjugated to the anti-ROR1 antibody or fragment by a chemical ligation process. In some embodiments, the linker is conjugated to the anti-ROR1 antibody or fragment by a native ligation. In some embodiments, the conjugation is as described in Dawson et al., Science (1994) 266:776-9; Dawson et al., J Am Chem Soc. (1997) 119:4325-9; Hackeng et al., PNAS (1999) 96:10068-73; or Wu et al., Angew Chem Int Ed. (2006) 45:4116-25. In some embodiments, the conjugation is as described in U.S. Pat. No. 8,936,910. In some embodiments, the linker is conjugated to the anti-ROR1 antibody or fragment either site-specifically or non-specifically via native ligation chemistry.

In some embodiments, the linker is conjugated to the anti-ROR1 antibody or antigen-binding fragment by a site-directed method utilizing a “traceless” coupling technology (Philochem). In some embodiments, the “traceless” coupling technology utilizes an N-terminal 1,2-aminothiol group on the binding moiety which is then conjugated with the antibody or fragment thereof containing an aldehyde group. See, e.g., Casi et al., JACS (2012) 134(13):5887-92. In some embodiments, the linker is conjugated to the anti-ROR1 antibody or fragment by a site-directed method utilizing an unnatural amino acid incorporated into the binding moiety. In some embodiments, the unnatural amino acid comprises p-acetylphenylalanine (pAcPhe). In some embodiments, the keto group of pAcPhe is selectively coupled to an alkoxy-amine derivatived conjugating moiety to form an oxime bond. See, e.g., Axup et al., PNAS (2012) 109(40):16101-6.

In some embodiments, the linker is conjugated to the anti-ROR1 antibody or antigen-binding fragment by a sis conjugated to the anti-ROR1 antibody by a site-directed method utilizing an enzyme-catalyzed process. In some embodiments, the site-directed method utilizes SMARTag™ technology (Redwood). In some embodiments, the SMARTag™ technology comprises generation of a formylglycine (FGly) residue from cysteine by formylglycine-generating enzyme (FGE) through an oxidation process under the presence of an aldehyde tag and the subsequent conjugation of FGly to an alkylhydraine-functionalized amino acid molecule via hydrazino-Pictet-Spengler (HIPS) ligation. See, e.g., Wu et al., PNAS (2009) 106(9):3000-5 and Agarwal et al., PNAS (2013) 110(1):46-51).

In some embodiments, the enzyme-catalyzed process comprises microbial transglutaminase (mTG). In certain embodiments, the linker is conjugated to the anti-ROR1 antibody or fragment by utilizing a microbial transglutaminase catalyzed process. In some embodiments, mTG catalyzes the formation of a covalent bond between the amide side chain of a glutamine within the recognition sequence and a primary amine of a functionalized amino acid molecule. In some embodiments, mTG is produced from Streptomyces mobarensis. See, e.g., Strop et al., Chemistry and Biology (2013) 20(2):161-7. In some embodiments, the linker is conjugated to the anti-ROR1 antibody by a carbohydrate-based chemical reaction. In the strategy of carbohydrate-based conjugation, the first step is usually to introduce new bioorthogonal functionalities to facilitate conjugation of the antibody to a drug. Strategies that can be used to introduce bioorthogonal functionalities onto the carbohydrate moiety for bioconjugation (glyco-conjugation) include but are not limited to chemical oxidation of glycans; enzymatic and chemo-enzymatic modification of glycans; and metabolic engineering of the carbohydrate moiety. Chemical approaches may use sodium periodate (NaIO4) to oxidize cis-glycol groups of e.g., galactose or sialic acid to generate aldehydes, which then can be coupled with hydrazide- or primary amine functionalized molecules to create acid-labeled hydrazones or with aminooxy groups to form oximes. Enzymatic and chemo-enzymatic approaches treat the sugar residue with neuraminidase (Neu) and galactose oxidase (Gal Oxi) to formate aldehyde functionalities. Continuous treatment of the antibody with β1,4-galactosyltransferase (Gal T)/α2,6-sialyltransferase (Sial T) can yield homogeneously sialylated antibodies. The resulting antibodies then can be selectively oxidized to the corresponding aldehyde functionalities, and if sialic acid derivatives are used, these antibodies can be used as selective bioorthogonal handles. Similarity, the antibody may be treated with β-galactosidase (Gal) and a mutant Gal T to mediate the attachment of bioorthogonal azide- or keto-galactoses to generate homogeneous G2 glycan patterns which possess non-natural functionalities. Another method makes non-canonical thio-fucose derivatives that can be incorporated into the glycan of antibodies by feeding the cells with the bioorthogonal sugar generating expressed antibodies that display thiol functionalities.

Linkers can be conjugated to the anti-ROR1 antibodies and antigen-binding fragments of the current disclosure in multiple ways. Generally, a linker and a cytotoxic moiety are synthesized and conjugated before attachment to an antibody. One method of attaching a linker-drug conjugate to an antibody involves reduction of solvent-exposed disulfides with dithiothreitol (DTT) or tris (2-carboxyethyl)phosphine (TCEP), followed by modification of the resulting thiols with maleimide-containing linker-drug moieties (e.g., 6-maleimidocaproyl-valine-citrulline-p-aminobenzyloxycarbonyl (MC-VC-PAB)). A native antibody contains 4 inter-chain disulfide bonds and 12 intra-chain disulfide bonds, as well as unpaired cysteines. Thus, antibodies modified in this way can comprise greater than one linker-drug moiety per antibody. In certain embodiments, the immunoconjugates described herein comprise at least 1, 2, 3, 4, 5, 6, 7, or 8, 9, or 10 linker/drug moieties. In certain embodiments, the immunoconjugates described herein comprise 1 to 10, 1 to 9, 1 to 8, 1 to 7, 1 to 6, 1 to 5, 1 to 4, 1 to 3, 1 to 2, linker/drug moieties, or 1 linker/drug moiety. In cases where the linker is branched and can each attach to multiple drug moieties, the ratio of the drug moiety to the antibody will be higher than using an unbranched linker.

The term “linkage,” as used herein, refer to a bond or chemical moiety formed from a chemical reaction between the functional group of one molecular entity and another molecule entity. Such bonds may include, but are not limited to, covalent and non-covalent bonds, while such chemical moieties may include, but are not limited to, esters, carbonates, carbamates, imines phosphate esters, hydrazones, acetals, orthoesters, peptide linkages, and oligonucleotide linkages. Hydrolytically stable linkage means that the linkage is substantially stable in water and does not react with water at useful pH values, including but not limited to under physiological conditions, for an extended period of time, perhaps even indefinitely. Hydrolytically unstable or degradable linkage means that the linkage is degradable in water or in aqueous solutions, including, for example, blood. Enzymatically unstable or degradable linkage means that the linkage can be degraded by one or more enzymes. By way of example only, PEG and related polymers may include degradable linkages in the polymer backbone or in the linker group between the polymer backbone and one or more of the terminal functional groups of the polymer molecule. Such degradable linkages include, but are not limited to, ester linkages formed by the reaction of PEG carboxylic acids or activated PEG carboxylic acids with alcohol groups on a biologically active agent, wherein such ester groups generally hydrolyze under physiological conditions to release the biologically active agent. Other hydrolytically degradable linkages include but are not limited to carbonate linkages; imine linkages resulted from reaction of an amine and an aldehyde; phosphate ester linkages formed by reacting an alcohol with a phosphate group; hydrazone linkages which are reaction product of a hydrazide and an aldehyde; acetal linkages that are the reaction product of an aldehyde and an alcohol; orthoester linkages that are the reaction product of a formate and an alcohol; peptide linkages formed by an amine group, including but not limited to, at an end of a polymer such as PEG, and a carboxyl group of a peptide; and oligonucleotide linkages formed by a phosphoramidite group, including but not limited to, at the end of a polymer, and a 5′ hydroxyl group of an oligonucleotide.

7. Exemplary ROR1 Immunoconjugates

In some embodiments, the ADC has the structure:

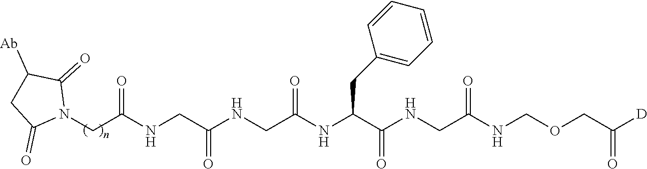

wherein Ab is an antibody or antigen-binding fragment thereof, n is an integer from 1-6, and D is an exatecan moiety or an analog thereof.

In some embodiments, the ADC has the structure:

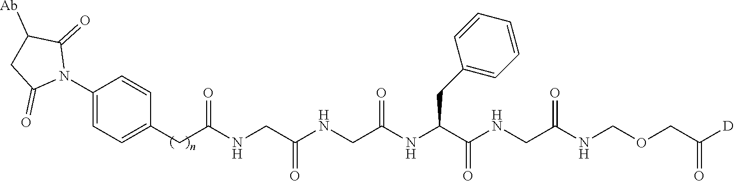

wherein Ab is an antibody or antigen-binding fragment thereof, n is an integer from 1-6, and D is an exatecan moiety or an analog thereof.

In some embodiments, the ADC has the structure:

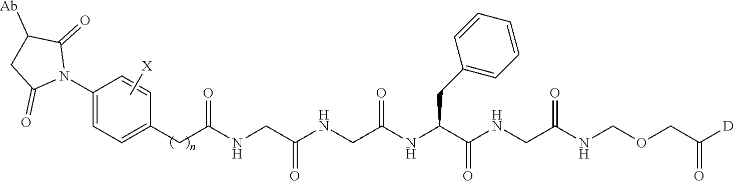

wherein Ab is an antibody or antigen-binding fragment thereof, X is an electron withdrawing group, n is an integer from 1-6, and D is an exatecan moiety or an analog thereof.

In some embodiments, the ADC has the structure:

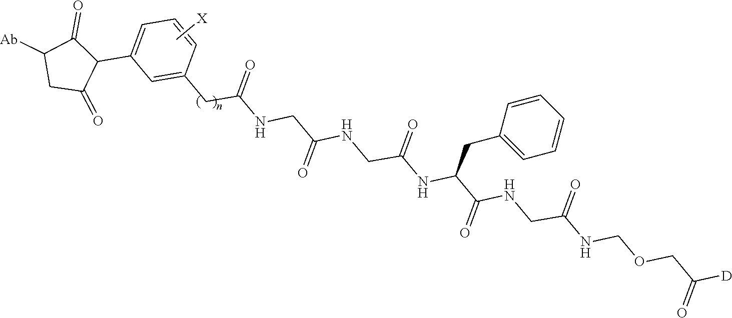

wherein Ab is an antibody or antigen-binding fragment thereof, X is an electron withdrawing group, n is an integer from 1-6, and D is an exatecan moiety or an analog thereof.

In some embodiments, the ADC has the structure:

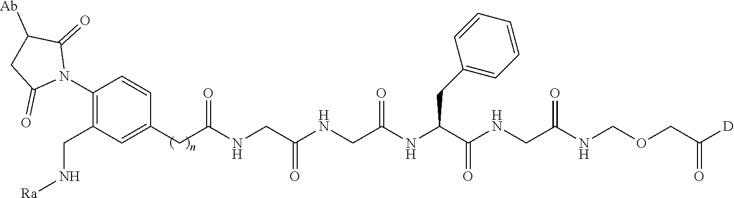

wherein Ab is an antibody or antigen-binding fragment thereof, Ra is H, Me or isopropyl, n is an integer from 1-6, and D is an exatecan moiety or an analog thereof.

In some embodiments, the ADC has the structure:

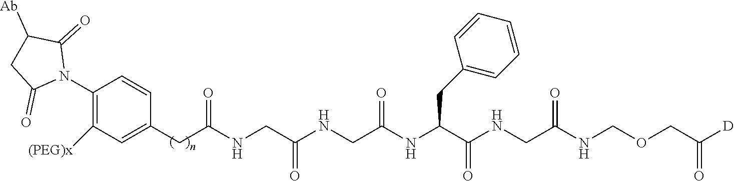

wherein Ab is an antibody or antigen-binding fragment thereof, x is an integer from 1-10, n is an integer from 1-6, and D is an exatecan moiety or an analog thereof.

In some embodiments, the ADC has the structure:

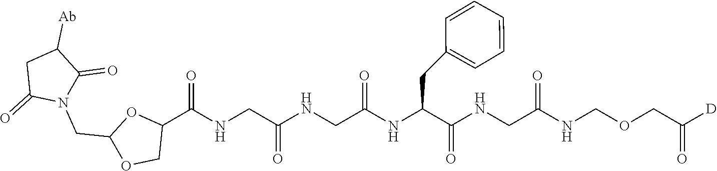

wherein Ab is an antibody or antigen-binding fragment thereof, and D is an exatecan moiety or an analog thereof.

In some embodiments, the ADC has the structure:

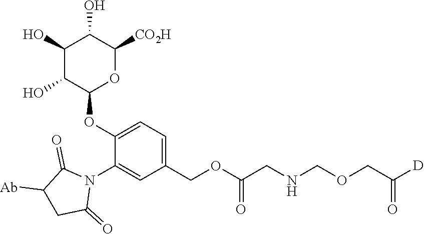

wherein Ab is an antibody or antigen-binding fragment thereof, and D is an exatecan moiety or an analog thereof.

In some embodiments, the ADC has the structure:

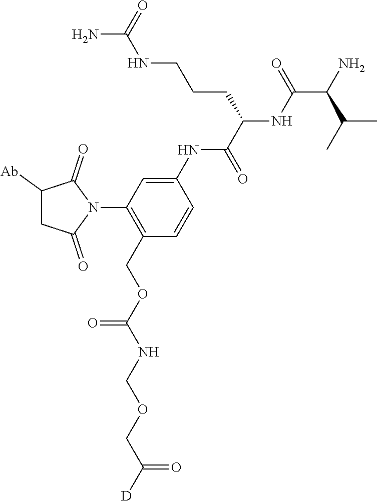

wherein Ab is an antibody or antigen-binding fragment thereof, and D is an exatecan moiety or an analog thereof.

In some embodiments, the ADC has the structure:

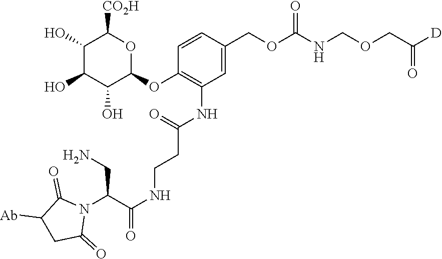

wherein Ab is an antibody or antigen-binding fragment thereof, and D is an exatecan moiety or an analog thereof.

In some embodiments, the ADC has the structure:

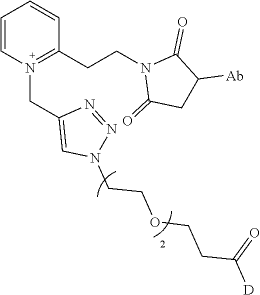

wherein Ab is an antibody or antigen-binding fragment thereof, and D is an exatecan moiety or an analog thereof.

In some embodiments, the ADC has the structure:

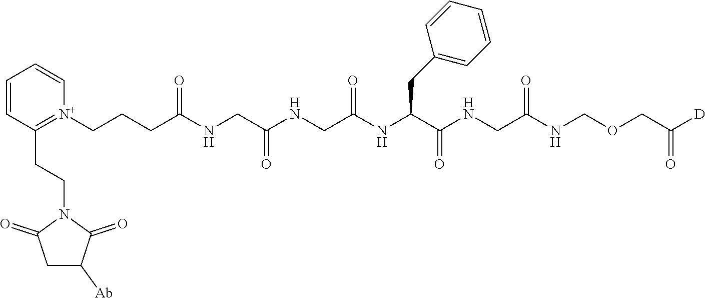

wherein Ab is an antibody or antigen-binding fragment thereof, and D is an exatecan moiety or an analog thereof.

In some embodiments, the ADC has the structure:

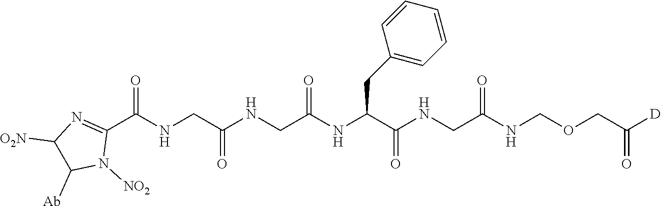

wherein Ab is an antibody or antigen-binding fragment thereof, and D is an exatecan moiety or an analog thereof.

In some embodiments, the ADC has the structure:

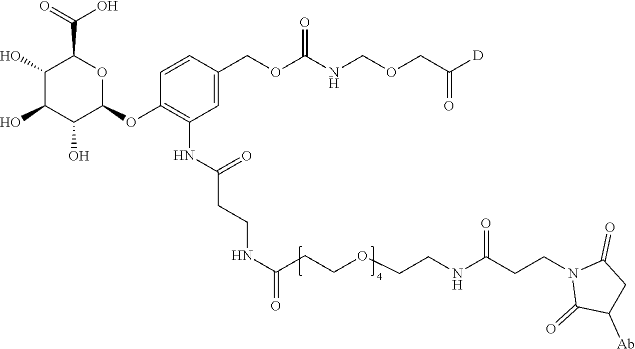

wherein Ab is an antibody or antigen-binding fragment thereof, and D is an exatecan moiety or an analog thereof.

In some embodiments, the ADC has the structure:

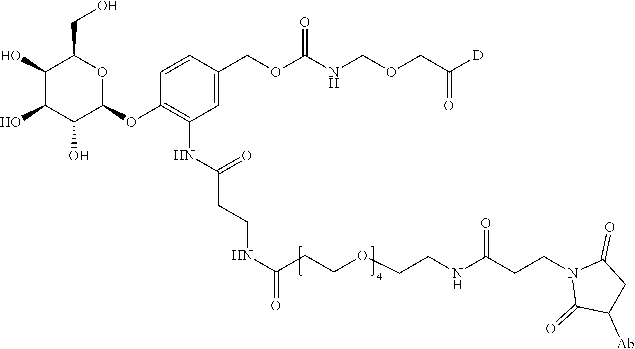

wherein Ab is an antibody or antigen-binding fragment thereof, and D is an exatecan moiety or an analog thereof.

In some embodiments, the ADC has the structure:

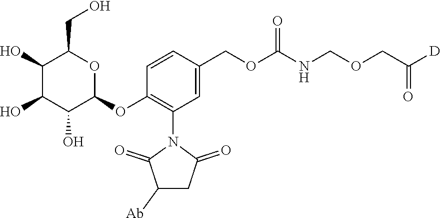

wherein Ab is an antibody or antigen-binding fragment thereof, and D is an exatecan moiety or an analog thereof.

In some embodiments, the ADC has the structure:

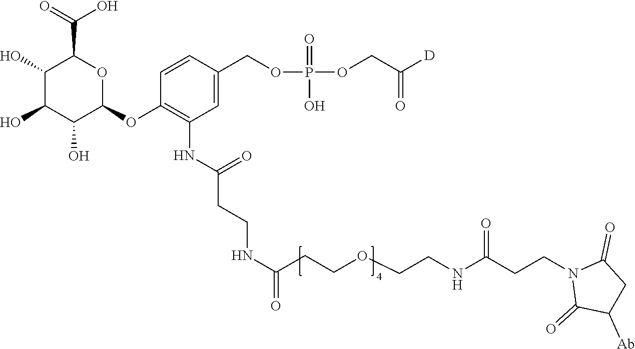

wherein Ab is an antibody or antigen-binding fragment thereof, and D is an exatecan moiety or an analog thereof.

In some embodiments, the ADC has the structure:

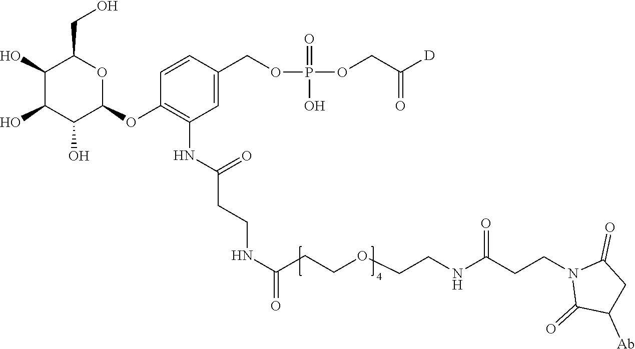

wherein Ab is an antibody or antigen-binding fragment thereof, and D is an exatecan moiety or an analog thereof.

In some embodiments, the ADC has the structure:

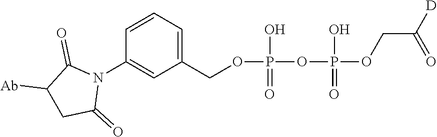

wherein Ab is an antibody or antigen-binding fragment thereof, and D is an exatecan moiety or an analog thereof.

In some embodiments, the ADC has the structure:

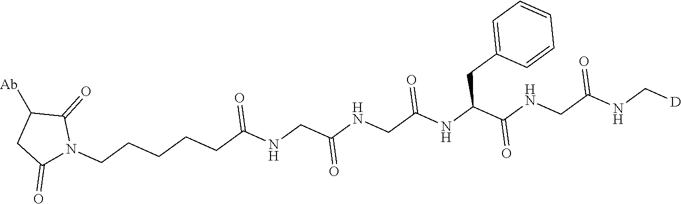

wherein Ab is an antibody or antigen-binding fragment thereof, and D is an exatecan moiety or an analog thereof.

In some embodiments, the ADC has the structure:

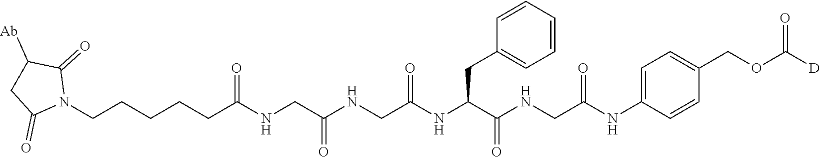

wherein Ab is an antibody or antigen-binding fragment thereof, and D is an exatecan moiety or an analog thereof.

In some embodiments, the ADC has the structure:

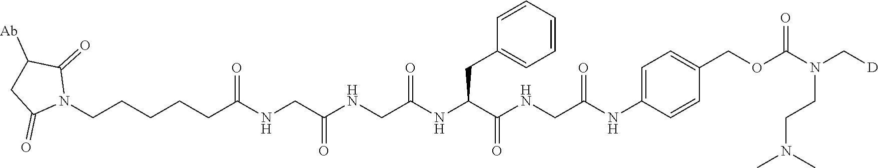

wherein Ab is an antibody or antigen-binding fragment thereof, and D is an exatecan moiety or an analog thereof.

In some embodiments, the ADC has the structure:

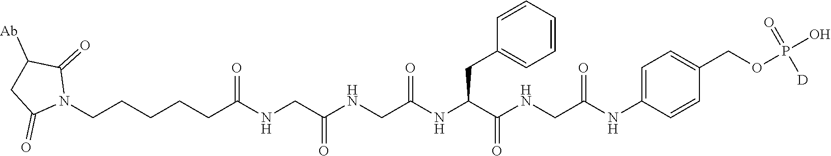

wherein Ab is an antibody or antigen-binding fragment thereof, and D is an exatecan moiety or an analog thereof.

In some embodiments, the ADC has the structure:

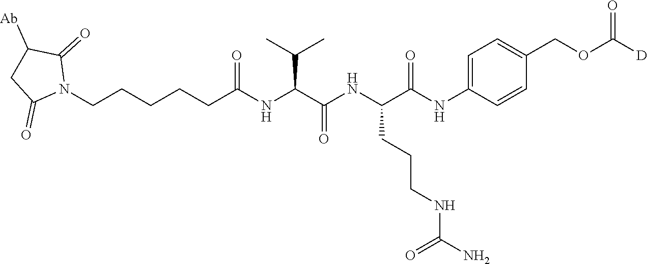

wherein Ab is an antibody or antigen-binding fragment thereof, and D is an exatecan moiety or an analog thereof.

In some embodiments, the ADC has the structure:

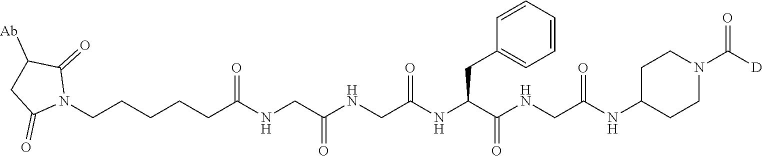

wherein Ab is an antibody or antigen-binding fragment thereof, and D is an exatecan moiety or an analog thereof.

In some embodiments, the ADC has the structure:

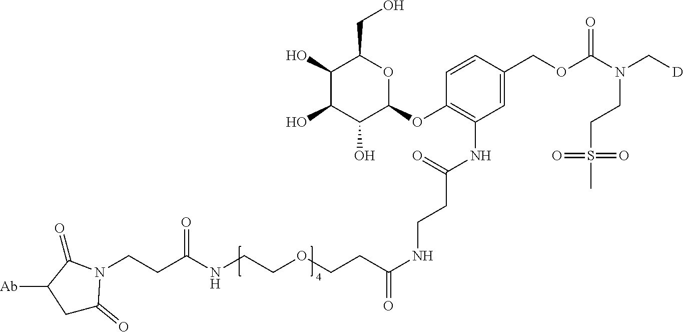

wherein Ab is an antibody or antigen-binding fragment thereof, and D is an exatecan moiety or an analog thereof.

In some embodiments, the ADC has the structure:

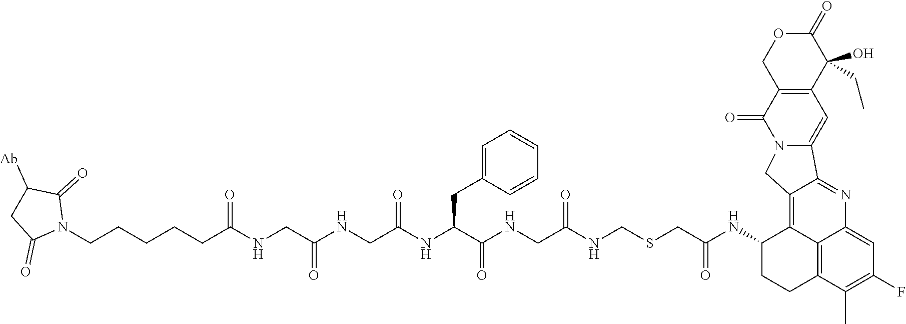

wherein Ab is an antibody or antigen-binding fragment thereof.

In some embodiments, the ADC has the structure:

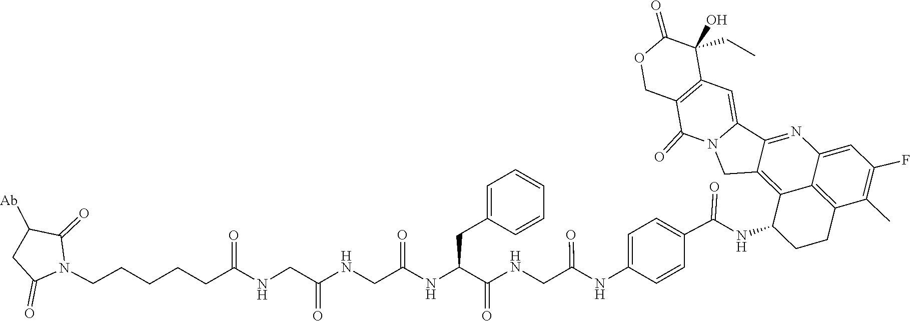

wherein Ab is an antibody or antigen-binding fragment thereof.

In some embodiments, the ADC has the structure:

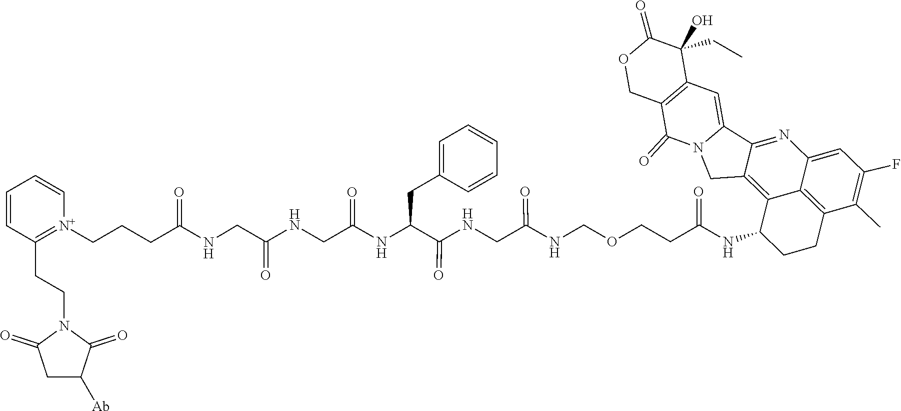

wherein Ab is an antibody or antigen-binding fragment thereof.

In some embodiments, the ADC has the structure:

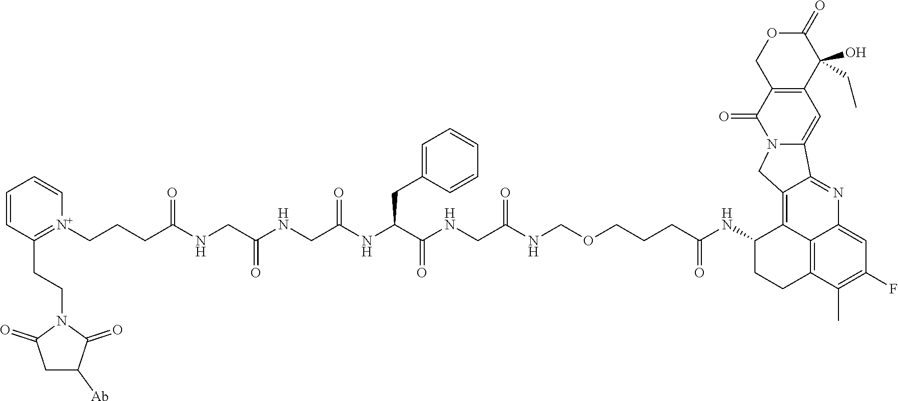

wherein Ab is an antibody or antigen-binding fragment thereof.

In some embodiments, the ADC has the structure:

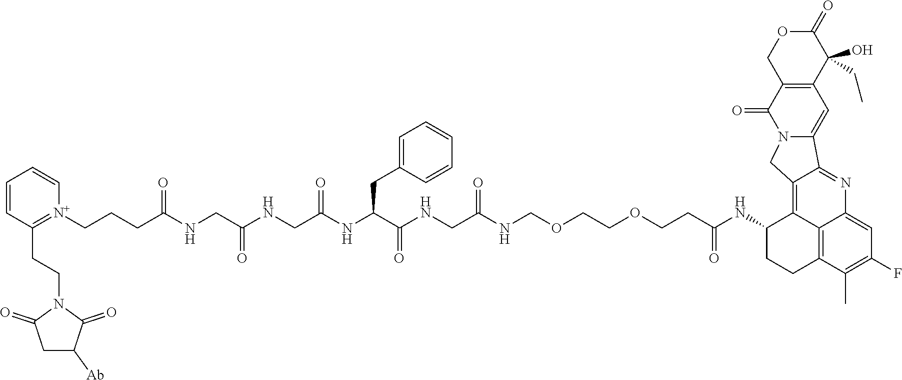

wherein Ab is an antibody or antigen-binding fragment thereof.

In some embodiments, the ADC has the structure:

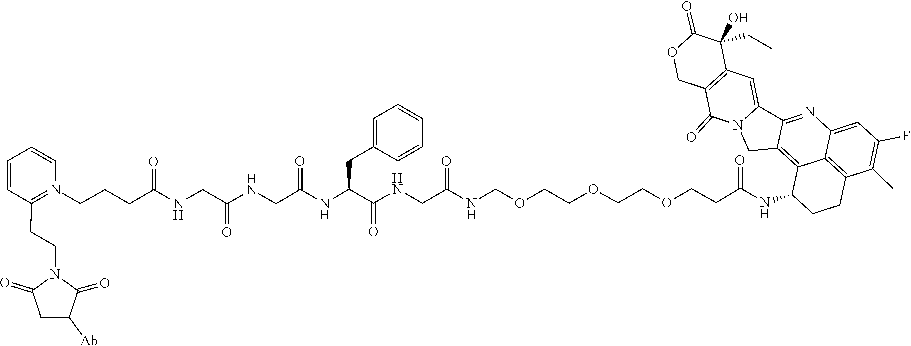

wherein Ab is an antibody or antigen-binding fragment thereof.

In some embodiments, the ADC has the structure:

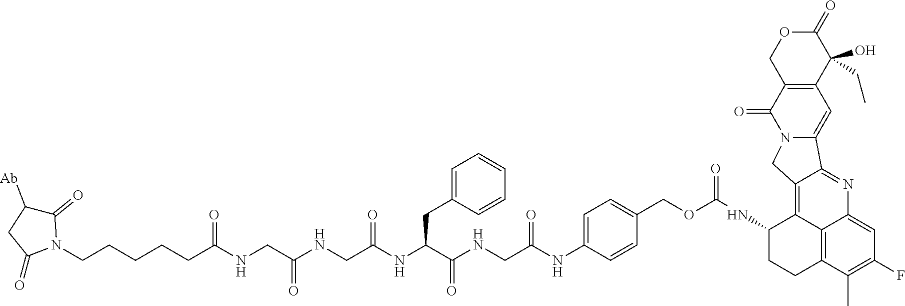

wherein Ab is an antibody or antigen-binding fragment thereof.

In some embodiments, the ADC has the structure:

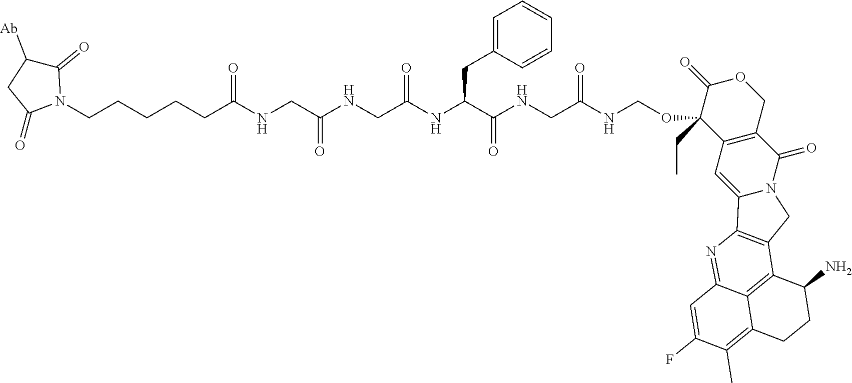

wherein Ab is an antibody or antigen-binding fragment thereof.

In some embodiments, the ADC has the structure:

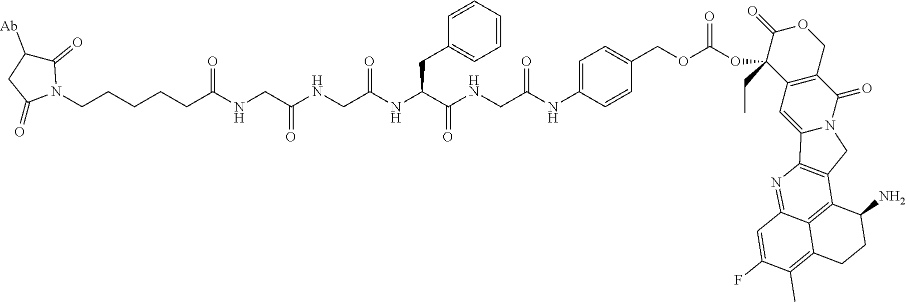

wherein Ab is an antibody or antigen-binding fragment thereof.

In some embodiments, the ADC has the structure:

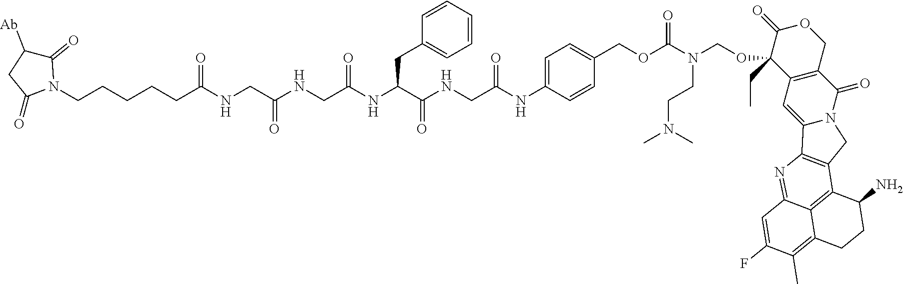

wherein Ab is an antibody or antigen-binding fragment thereof.

In some embodiments, the ADC has the structure:

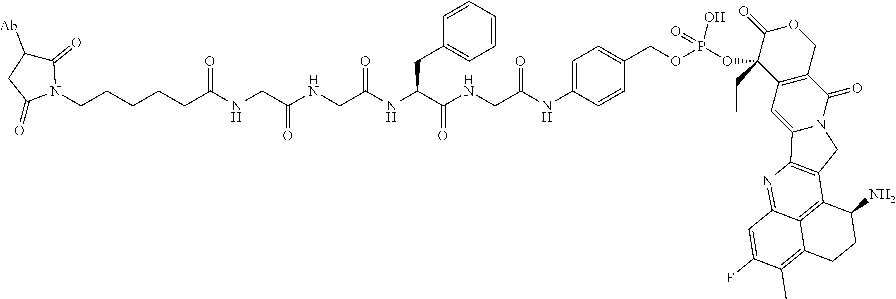

wherein Ab is an antibody or antigen-binding fragment thereof.

In some embodiments, the ADC has the structure:

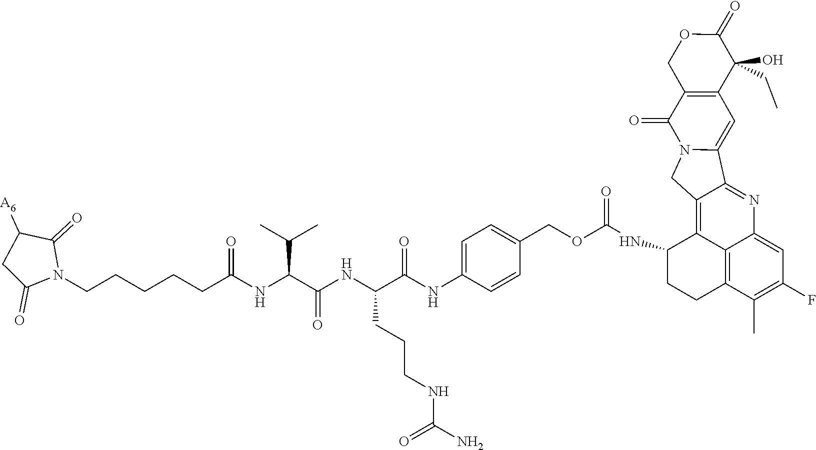

wherein Ab is an antibody or antigen-binding fragment thereof.

In some embodiments, the ADC has the structure:

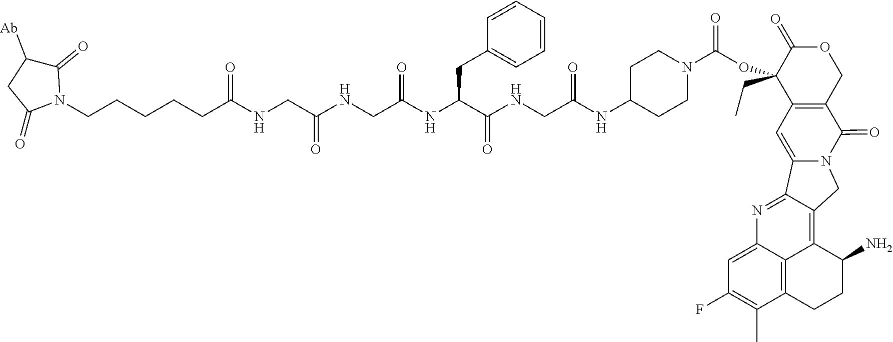

wherein Ab is an antibody or antigen-binding fragment thereof.

In some embodiments, the ADC has the structure:

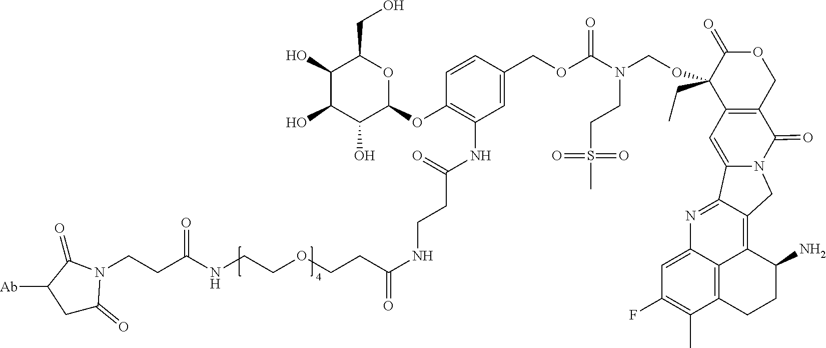

wherein Ab is an antibody or antigen-binding fragment thereof.

Further exemplary immunoconjugates of the present invention are shown in the table below:

TABLE 2
Exemplary Immunoconjugates

| | | | | | | Target |
|---|---|---|---|---|---|---|
| Construct | Conjugation | Linker Components | | | Payload | DAR |
| ADC-U | MAL | —C5—(C═O)— | GGFG | Spacer* | exatecan | 1-4 |
| ADC-V | MAL | —C5—(C═O)— | GGFG | Spacer* | exatecan | 5-8 |
| ADC-W | MAL | —Ph—C3—(C═O)— | GGFG | Spacer* | exatecan | 1-4 |
| ADC-X | MAL | —Ph—C3—(C═O)— | GGFG | Spacer* | exatecan | 5-8 |

In the above table, the abbreviations are used as follows:
MAL=maleimide chemistry;
C5=—(CH₂)₅—;
GGFG (SEQ ID NO:55)=tetrapeptide glycine-glycine-phenylalanine-glycine;
-Ph-C3=phenyl-CH₂—CH₂—CH₂—; and
Spacer*=—(NH)—CH₂—O—CH₂—(C═O)—.

The chemical structures of immunoconjugates in Table 3 are shown in the table below. The difference between ADC-U and ADC-V and between ADC-W and ADC-X lies in the DAR (see Example 1 below).

TABLE 3
Chemical Structures of Exemplary Immunoconjugates

| ADC | Structure |
|---|---|
| U/V | 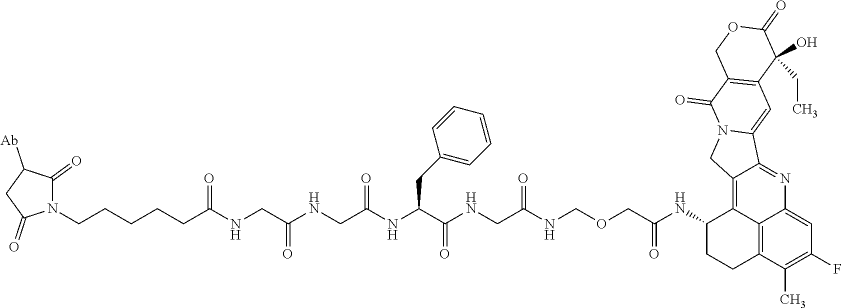 |
| W/X | 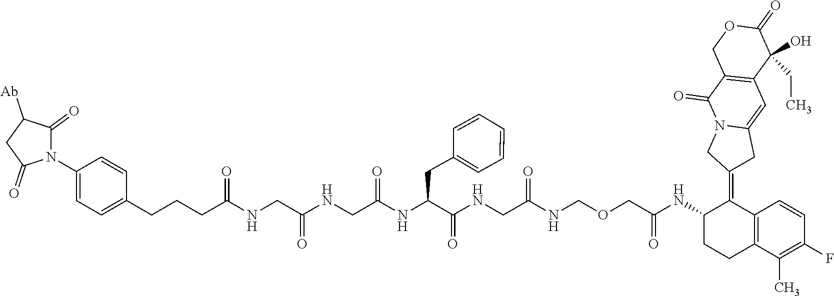 |

### II. Synthesis of Immunoconjugates

Exemplary synthesis methods for the ADCs shown in Table 3 are illustrated in Example 1 below. In ADC-U, -V, -W, and -X, the antibody is covalently bonded to the linker/payload moiety via cysteine residues.

### III. Immunoconjugate Therapy

The immunoconjugates described herein are useful for treating a variety of cancers. ROR1 has been shown to express across many types of tumors, including lymphomas and solid tumors. High proportions of human cancers express ROR1. For example, Zhang et al. showed that 54% ovarian cancers, 57% colon cancers, 77% lung cancers, 90% lymphomas, 89% skin cancers, 83% pancreatic cancers, 73% testicular cancers, 43% bladder cancers, 96% uterus cancers, 90% prostate cancers, and 83% adrenal cancers that they examined had moderate-to-strong staining with the anti-ROR1 antibody 4A5 (Zhang et al., Am J Pathol. (2012) 181(6):1903-10). Daneshmanesh et al. similarly found near universal expression of ROR1 in CLL and hairy cell leukemia (HCL) and varying degrees of expression in other lymphoid cancers such as mantle cell lymphoma (MCL), diffuse large B-cell lymphoma (DLBCL)/marginal zone lymphoma (MZL), follicular lymphoma (FL), chronic myeloid leukemia (CML), acute myeloid lymphoma (AML), and myeloma (Daneshmanesh et al., Leuk Lymphoma (2013) 54(4):843-50). Our own studies similarly have shown that substantial proportions of patients with hepatocellular cancers (HCC) or non-small-cell lung cancer (NSCLC) are ROR1-positive. This broad tumor expression pattern of ROR1 renders the immunoconjugates of the present invention useful in treating many hematological cancers and solid tumors, such as those aforementioned. Further, it has been shown that ROR1 expression increases in aggressive cancers and correlates with poor prognosis; thus, immunoconjugates of the present invention are particularly well suited to treat aggressive or advanced cancers. In some embodiments, the immunoconjugates of the invention lead to partial or complete tumor regression. In particular embodiments, the partial or complete tumor regression may be sustained beyond the final dose of immunoconjugate treatment.

The ROR1 immunoconjugates of the invention, such as those made with an antibody that binds to a ROR1 epitope set forth in SEQ ID NO:1 or 2 (e.g., Ab1, Ab2, Ab3, or Ab4), are effective in treating cancers such as solid tumors that are heterogeneous in ROR1 expression. Tumors having as little as 20% of their cells expressing ROR1 can be treated effectively by the ROR1 immunoconjugates; for example, the tumors may have 20% or more, 30% or more, 40% or more, 50% or more, 60% or more, or 70% or more of their cells expressing ROR1. Without wishing to be bound by theory, it is contemplated that the ROR1 immunoconjugates of the invention may cause cell death in ROR1-negative tumor cells through bystander toxicity effect (i.e., the payload released from a dead tumor cell causes cytotoxicity to a neighboring tumor cell), or by enhancing the anti-tumor immunity of the immune system, or both.

“Treat”, “treating” and “treatment” refer to a method of alleviating or abrogating a biological disorder and/or at least one of its attendant symptoms. As used herein, to “alleviate” a disease, disorder or condition means reducing the severity and/or occurrence frequency of the symptoms of the disease, disorder, or condition. Further, references herein to “treatment” include references to curative, palliative and prophylactic treatment. Treatment of cancer encompasses inhibiting cancer growth (including causing partial or complete cancer regression), inhibiting cancer progression or metastasis, preventing cancer recurrence or residual disease, and/or prolonging the patient's survival.

In some embodiments, the cancer treatable by the immunoconjugates described herein is a ROR1-expressing cancer. The ROR1-expressing cancer can be determined by any suitable method of determining gene or protein expression, for example, by histology, flow cytometry, RT-PCR, or RNA-Seq. The cancer cells used for the determination may be obtained through tumor biopsy or through collection of circulating tumor cells. In certain embodiments, if an antibody-based assay such as flow cytometry or immunohistochemistry is used, ROR1-expressing cancers are any cancers with cells that show anti-ROR1 antibody reactivity greater than that of an isotype control antibody. In certain embodiments, if an RNA-based assay is used, ROR1-expressing cancers are those that show an elevated level of ROR1 RNA compared to a negative control cell or cancer that does not express ROR1.

In certain embodiments, the antibodies and immunoconjugates are for use in treating hematological malignancies. In certain embodiments, the antibodies and immunoconjugates are for use in treating solid tumors. The cancer to be treated may be selected from, e.g., lymphoma, small lymphocytic lymphoma, marginal zone lymphoma, marginal cell B-cell lymphoma, Burkitt's lymphoma, mantle cell lymphoma, follicular lymphoma, diffuse large B-cell lymphoma, a non-Hodgkin lymphoma that has undergone Richter's transformation, T cell non-Hodgkin lymphoma, lymphoplasmacytoid lymphoma, Waldenström macroglobulinemia, acute lymphoblastic leukemia, acute myeloid leukemia, chronic lymphocytic leukemia, small lymphocytic leukemia, T cell leukemia, sarcoma, osteosarcoma, Ewing sarcoma, renal cell carcinoma, hepatocellular carcinoma, colon cancer, colorectal cancer, breast cancer, epithelial squamous cell cancer, glioblastoma, melanoma, myeloma, multiple myeloma, stomach cancer, brain cancer, lung cancer, non-small cell lung cancer, pancreatic cancer, cervical cancer, ovarian cancer, liver cancer, bladder cancer, prostate cancer, testicular cancer, thyroid cancer, and head and neck cancer. In certain embodiments, the cancer to be treated can be a cancer that is refractory to other therapeutics (for example, triple negative breast cancer).

In certain embodiments, the methods for treating cancer described herein comprise treatment with an immunoconjugate of the invention and treatment with an additional therapeutic agent or biologically active molecule. Examples of biologically active molecules include, but are not limited to, peptides, proteins, enzymes, small molecule drugs, prodrugs, carbohydrates, imaging agents, lipids, nucleosides, radionuclides, oligonucleotides, toxins, cells, antibiotics, fungicides, anti-viral agents, anti-inflammatory agents, anti-tumor agents, cardiovascular agents, anti-anxiety agents, hormones, growth factors, steroidal agents, microbially derived toxins, and the like.

Further examples of biologically active molecules include, but are not limited to: NCA1, auristatin, auristatin E, DNA minor groove binding agents, DNA minor groove alkylating agents, enediyne, lexitropsin, duocarmycin, taxane, puromycin, dolastatin, maytansinoid, vinca alkaloid, AFP, MMAF, MMAE, AEB, AEVB, taxoids (e.g., paclitaxel and paclitaxel derivatives (TAXOL®, Bristol-Myers Squibb Oncology, Princeton, N.J.), ABRAXANE® (American Pharmaceutical Partners, Schaumberg, Ill.), as well as docetaxel and docetaxel derivatives), CC-1065, SN-38, topotecan, morpholino-doxorubicin, rhizoxin, cyanomorpholino-doxorubicin, dolastatin-10, echinomycin, combretatstatin, chalicheamicin, maytansine, DM-1, netropsin, podophyllotoxin (e.g., etoposide and teniposide), baccatin and its derivatives, anti-tubulin agents, cryptophysin, combretastatin, vincristine, vincristine sulfate, vinblastine, vindesine, vinorelbine, VP-16, camptothecin, epothilone A, epothilone B, nocodazole, colchicines, colcimid, estramustine, cemadotin, discodermolide, eleutherobin, mechlorethamine, cyclophosphamide, melphalan, carmustine, lomustine, semustine, streptozocin, chlorozotocin, uracil mustard, chlormethine, chlorambucil, pipobroman, triethylenemelamine, triethylenethiophosphoramine, busulfan, dacarbazine, temozolomide, ytarabine, cytosine arabinoside, fluorouracil, 5-fluorouracil (5-FU), floxuridine, 6-thioguanine, 6-mercaptopurine, pentostatin, methotrexate, 10-propargyl-5,8-dideazafolate, 5,8-dideazatetrahydrofolic acid, leucovorin, fludarabine phosphate, pentostatine, gemcitabine, Ara-C, deoxycoformycin, mitomycins such as mitomycin-C, L-asparaginase, azathioprine, brequinar, antibiotics (e.g., anthracycline, gentamicin, cefalotin, vancomycin, telavancin, daptomycin, azithromycin, erythromycin, rocithromycin, furazolidone, amoxicillin, ampicillin, carbenicillin, flucloxacillin, methicillin, penicillin, ciprofloxacin, moxifloxacin, ofloxacin, doxycycline, minocycline, oxytetracycline, tetracycline, streptomycin, rifabutin, ethambutol, and rifaximin), enediyne antibiotics (e.g., calicheamicin, calicheamicin gammaII and calicheamicin omegaII, and dynemicin, including dynemicin A), antiviral drugs (e.g., abacavir, acyclovir, ampligen, cidofovir, delavirdine, didanosine, efavirenz, entecavir, fosfonet, ganciclovir, ibacitabine, immunovir, idoxuridine, inosine, lopinavir, methisazone, nexavir, nevirapine, oseltamivir, penciclovir, stavudine, trifluridine, truvada, valaciclovir, and zanamivir), daunorubicin hydrochloride, daunoriycin, rubidomycin, cerubidine, idarubicin, doxorubicin, epirubicin and morpholino derivatives, phenoxizone biscyclopeptides (e.g., dactinomycin), basic glycopeptides (e.g., bleomycin), anthraquinone glycosides (e.g., plicamycin and mithramycin), anthracenediones (e.g., mitoxantrone), azirinopyrrolo indolediones (e.g., mitomycin), macrocyclic immunosuppressants (e.g., cyclosporine, FK-506, tacrolimus, prograf, and rapamycin), navelbene, CPT-11, anastrazole, letrazole, capecitabine, reloxafine, droloxafine, allocolchicine, Halichondrin B, colchicine and colchicine derivatives, rhizoxin, thiocolchicine, trityl cysterin, vinblastine sulfate, hydroxyurea, N-methylhydrazine, epidophyllotoxin, procarbazine, mitoxantrone, leucovorin, and tegafur. “Taxanes” include paclitaxel, as well as any active taxane derivative or pro-drug. Chemotherapeutic agents such as erlotinib (TARCEVA®, Genentech/OSI Pharm.), bortezomib (VELCADE®, Millenium Pharm.), fulvestrant (FASLODEX®, AstraZeneca), sunitinib (Sutent®, Pfizer), letrozole (FEMARA®, Novartis), imatinib mesylate (GLEEVEC®, Novartis), PTK787/ZK 222584 (Novartis), oxaliplatin (Eloxatin®, Sanofi), leucovorin, lapatinib (TYKERB®, GSK572016, GlaxoSmithKline), lonafamib (SCH 66336), sorafenib (BAY43-9006, Bayer Labs.), and gefitinib (IRESSA®, AstraZeneca), AG1478, AG1571 (SU 5271; Sugen), alkylating agents such as thiotepa and cyclosphosphamide (CYTOXAN®); alkyl sulfonates such as busulfan, improsulfan and piposulfan; antifolate antineoplastic such as pemetrexed (ALIMTA® Eli Lilly); aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (such as bullatacin and bullatacinone); a camptothecin (including the synthetic analogue topotecan); bryostatin; callystatin; CC-1065 (including its synthetic analogues adozelesin, carzelesin and bizelesin); cryptophycins (such as cryptophycin 1 and cryptophycin 8); dolastatin; duocarmycin (including its synthetic analogues KW-2189 and CBT-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosoureas such as carmustine, chlorozotocin, fotemustine, lomustine, nimustine, and ranimnustine; bisphosphonates, such as clodronate; an esperamicin; as well as neocarzinostatin chromophore and related chromoprotein enediyne antibiotic chromophores, aclacinomysins, actinomycin, anthramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (ADRIAMYCIN®) (including morpholino-doxorubicin, cyanomorpholino-doxorubicin, 2-pyrrolino-doxorubicin and deoxydoxorubicin), epirubicin, esorubicin, idarubicin, marcellomycin, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-FU; folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel (TAXOL®, Bristol-Myers Squibb Oncology, Princeton, N.J.), ABRAXANE™ Cremophor-free, albumin, nanoparticle formulation of paclitaxel (American Pharmaceutical Partners, Schaumberg, Ill.), and TAXOTERE® doxetaxel (Rhone-Poulenc Rorer, Antony, France); chlorambucil; GEMZAR® gemcitabine; 6-thioguanine; mercaptopurine; methotrexate; platinum analogs such as cisplatin and carboplatin; vinblastine; platinum; etoposide (VP-16); mitoxantrone; NAVELBINE® vinorelbine; novantrone; teniposide; edatrexate; daunomycin; aminopterin; xeloda; ibandronate; topoisomerase inhibitor RFS 2000; difluoromethylornithine (DMFO); retinoids such as retinoic acid; and pharmaceutically acceptable salts, esters, acids, prodrugs, or derivatives of any of the above.

In certain embodiments, the immunoconjugate and the additional therapeutic agent or biologically active molecule are administered at the same time, e.g., in the same formulation. In certain embodiments, they are administered separately, on the same or different dosing schedules. In some embodiments, the additional therapeutic agent is a vascular endothelial growth factor (VEGF) inhibitor, a Bruton's tyrosine kinase (BTK) inhibitor, an inhibitor of the mammalian target of rapamycin (mTOR), a phosphoinositide 3-kinase (PI3K) inhibitor, a Janus kinase/signal transducers and activators of transcription (Jak/STAT) signaling inhibitor, a B-cell lymphoma 2 (Bcl-2) inhibitor, a spleen tyrosine kinase (SYK) inhibitor, a microtubule inhibitor, an epithelial growth factor receptor (EGFR) inhibitor, a poly ADP ribose polymerase (PARP) inhibitor, an anaplastic lymphoma kinase (ALK) inhibitor, a DNA-repair inhibitor, a DNA cross-linker, a nucleoside analog, or an immunomodulatory agent.

In some embodiments, the additional therapeutic agent is
a) an antibody such as rituximab (anti-CD20) or bevacizumab (anti-VEGF);
b) a Bruton's tyrosine kinase inhibitor such as acalabrutinib or ibrutinib;
c) an mTOR inhibitor such as sapanisertib, everolimus or BEZ235;
d) a PI3K inhibitor such as idelalisib or buparlisib;
e) a Jak/STAT signaling inhibitor such as ruxolitinib;
f) a Bcl-2 inhibitor such as ABT-199/venetoclax, Bcl-2i-1, or Bcl-2i-2;
g) a SYK inhibitor such as fostamatinib;
h) a microtubule inhibitor such as paclitaxel or vincristine;
i) an EGFR inhibitor such as erlotinib;
j) a PARP inhibitor such as olaparib;
k) an ALK inhibitor such as crizotinib;
l) a DNA-repair inhibitor such as carboplatin;
m) a DNA cross-linker such as oxaliplatin/cisplatin;
n) a nucleoside analog such as gemcitabine; or
o) an immunomodulatory drug (IMiD) such as lenalidomide or pomalidomide.

In certain embodiments, an immunoconjugate of the invention and an additional therapeutic agent or biologically active molecule are used in combination to treat CLL, MCL, or a non-Hodgkin lymphoma that has undergone Richter's transformation. In particular embodiments, the additional therapeutic agent or biologically active molecule is, e.g., ibrutinib, acalabrutinib, venetoclax, Bcl-2i-1, Bcl-2i-2, everolimus, sapanisertib, or idelalisib.

Additional examples of the additional therapeutic agent are pacritinib, buparlisib, BEZ235, ruxolitinib, fostamatinib, rituximab, lenalidomide, pomalidomide, paclitaxel, vincristine, erlotinib, crizotinib, carboplatin, oxaliplatin/cisplatin, bevacizumab, and gemcitibine.

In certain embodiments, an immunoconjugate of the invention is used in combination with an immune checkpoint modulator that enhances the patient's immune system. For example, the conjugate is used with an immune checkpoint inhibitor such as an antibody or antibody derivative, an antisense oligonucleotide, a small interfering RNA, an aptamer, or a peptide, targeting programmed death-ligand 1 (PD-L1, also known as B7-H1, CD274), programmed death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, ID02, ICOS (inducible T cell costimulator), KIR, LAIR1, LIGHT, MARCO (macrophage receptor with collagenous structure), PS (phosphatidylserine), OX-40, SLAM, TIGHT, VISTA, VTCN1, or any combination thereof.

It is understood that the immunoconjugates of the invention may be used in a method of treatment as described herein, may be for use in a treatment as described herein, and/or may be for use in the manufacture of a medicament for a treatment as described herein. The invention also provides kits and articles of manufacture comprising the immunoconjugates of the invention as described herein.

### IV. Pharmaceutical Compositions

In some embodiments, the immunoconjugate of the invention may be comprised in a pharmaceutical composition further comprising one or more pharmaceutically acceptable excipients, carriers, and diluents. For example, the antibodies and immunoconjugates of the invention may be administered suspended in a sterile solution (e.g., a solution comprising 0.9% NaCl). In certain embodiments, the solution further comprises one or more of the following: buffers (e.g., acetate, citrate, histidine, succinate, phosphate, bicarbonate and hydroxymethylaminomethane (Tris) buffers); surfactants (e.g., polysorbate 80 (Tween 80), polysorbate 20 (Tween 20), and poloxamer 188); polyols/disaccharide/polysaccharides (e.g., glucose, dextrose, mannose, mannitol, sorbitol, sucrose, trehalose, and dextran 40); amino acids (e.g., glycine and arginine); antioxidants (e.g., ascorbic acid and methionine); and/or chelating agents (e.g., EDTA and EGTA). Any combination of these excipients is also contemplated. In certain embodiments, the immunoconjugates of the invention are shipped/stored lyophilized and reconstituted before administration. In certain embodiments, lyophilized antibody or immunoconjugate formulations comprise a bulking agent such as mannitol, sorbitol, sucrose, trehalose, and/or dextran 40. The lyophilized formulation can be contained in a vial, such as a glass vial. The immunoconjugates, when formulated, whether reconstituted or not, can be buffered at a certain pH, e.g., less than 7.0 (such as a pH between 4.5 and 6.5, between 4.5 and 6.0, between 4.5 and 5.5, between 4.5 and 5.0, or between 5.0 and 6.0).

The immunoconjugates of the invention, as used herein, encompass pharmaceutically acceptable salts or esters of the conjugates. The pharmaceutically acceptable salts may be formed when an acidic proton present in the polypeptides either is replaced by a metal ion, by way of example an alkali metal ion, an alkaline earth ion, or an aluminum ion; or coordinates with an organic base. In addition, the salt forms of the immunoconjugates can be prepared using salts of the starting materials or intermediates. The immunoconjugates described herein may be prepared as pharmaceutically acceptable acid addition salts (which are a type of a pharmaceutically acceptable salt) by reacting the free base form of the polypeptides described herein with a pharmaceutically acceptable inorganic or organic acid. Alternatively, the immunoconjugates described herein may be prepared as pharmaceutically acceptable base addition salts (which are a type of a pharmaceutically acceptable salt) by reacting the free acid form of amino acids in polypeptides described herein with a pharmaceutically acceptable inorganic or organic base.

Pharmaceutically acceptable salts include, but are not limited to: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; and (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate, sodium hydroxide, and the like. The corresponding counterions of the pharmaceutically acceptable salts may be analyzed and identified using various methods including, but not limited to, ion exchange chromatography, ion chromatography, capillary electrophoresis, inductively coupled plasma, atomic absorption spectroscopy, mass spectrometry, or any combination thereof.

In some embodiments, a pharmaceutical composition of the invention includes multiparticulate formulations. In some embodiments, the pharmaceutical composition includes nanoparticle formulations. In some embodiments, nanoparticles comprise cMAP, cyclodextrin, and/or lipids. In some cases, nanoparticles comprise solid lipid nanoparticles, polymeric nanoparticles, self-emulsifying nanoparticles, liposomes, microemulsions, and/or micellar solutions. Additional exemplary nanoparticles include, but are not limited to, paramagnetic nanoparticles, superparamagnetic nanoparticles, metal nanoparticles, fullerene-like materials, inorganic nanotubes, dendrimers (such as with covalently attached metal chelates), nanofibers, nanohoms, nano-onions, nanorods, nanoropes and/or quantum dots. In some embodiments, a nanoparticle is a metal nanoparticle, e.g., a nanoparticle of scandium, titanium, vanadium, chromium, manganese, iron, cobalt, nickel, copper, zinc, yttrium, zirconium, niobium, molybdenum, ruthenium, rhodium, palladium, silver, cadmium, hafnium, tantalum, tungsten, rhenium, osmium, iridium, platinum, gold, gadolinium, aluminum, gallium, indium, tin, thallium, lead, bismuth, magnesium, calcium, strontium, barium, lithium, sodium, potassium, boron, silicon, phosphorus, germanium, arsenic, antimony, and combinations, alloys or oxides thereof.

Any method for administering immunoconjugates accepted in the art may be employed. The pharmaceutical compositions of the invention are typically suitable for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intratumoral, and intrasynovial injection or infusions; and kidney dialytic infusion techniques. Regional perfusion is also contemplated. Particular embodiments include the intravenous and subcutaneous routes.

### V. Articles of Manufacture and Kits

The present invention also provides articles of manufacture, e.g., kits, comprising a container (e.g., a single-use or multi-use container) containing a pharmaceutical composition of the present immunoconjugate), optionally an additional biologically active molecule (e.g., another therapeutic agent), and instructions for use. The immunoconjugate and additional biologically active molecule can be packaged separately in suitable packing such as a vial or ampule made from non-reactive glass or plastic. In certain embodiments, the vial or ampule holds lyophilized powder comprising the immunoconjugate or additional therapeutic agent or biologically active molecule. In certain embodiments, the vial or ampule holds a concentrated stock (e.g., 2×, 5×, 10× or more) of the immunoconjugate or biologically active molecule. In certain embodiments, the articles of manufacture such as kits include a medical device for administering the immunoconjugate and/or biologically active molecule (e.g., a syringe and a needle); and/or an appropriate diluent (e.g., sterile water and normal saline). The present invention also includes methods for manufacturing said articles.

Unless the context requires otherwise, throughout the specification and claims, the word “comprise” and variations thereof, such as, “comprises” and “comprising,” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. As used herein the term “about” refers to a numerical range that is 10%, 5%, or 1% plus or minus from a stated numerical value within the context of the particular usage. Further, headings provided herein are for convenience only and do not interpret the scope or meaning of the claimed embodiments. All publications and patents mentioned herein are incorporated herein by reference in their entirety for the purpose of describing and disclosing, for example, the constructs and methodologies that are described in the publications, which might be used in connection with the presently described inventions. The publications discussed herein are provided solely for their disclosure prior to the filing date of the present application. Nothing herein is to be construed as an admission that the inventors of the subject invention are not entitled to antedate such disclosure by virtue of prior invention or for any other reason. Unless defined otherwise, all technical and scientific terms used herein have the same meaning as commonly understood to one of ordinary skill in the art to which the inventions described herein belong. Any methods, devices, and materials similar or equivalent to those described herein can be used in the practice or testing of the inventions described herein.

### EXAMPLES

The following examples illustrate representative embodiments of the present invention and are not meant to be limiting in any way.

### Example 1: Synthesis of Exemplary Immunoconjugates

Conjugation of Ab1 cysteines in ADC-U, ADC-V, ADC-W, and ADC-X

Conjugation of Ab1 with MC-VC-PAB-MMAE (ADC-A) was performed at multiple scales (2 mg, 30 mg, and 350 mg) with similar results. For the smallest scale, 2 mg Ab1 (10 mg/mL in PBS, pH 6.5) was treated with 2.50 eq of tris(2-carboxyethyl)phosphine (TCEP, 5 mM) in conjugation buffer solution in a water bath at 37° C. for 2 h. PBS is 15.75 mM Na₂HPO₄, 34.25 mM NaH₂PO₄, 2 mM EDTA, 50 mM NaCl, pH 6.5. Subsequently, the reaction was cooled to 4° C. Next, 7 eq MC-VC-PAB-MMAE in N,N-dimethylacetamide (DMA) was added and the mixture was left at 4° C. for an additional 1 hour. The buffer was exchanged with 20 mM histidine, pH 5.5 (MMAE buffer) by using a spin desalting column (40 kD, 0.5 mL). The number of drug molecules linked per antibody molecule (DAR) was determined using RP-HPLC and is summarized in Table 4. See also WO 2018/237335.

In a similar fashion, other linker/payloads (ADC-U, -V, -W, and -X; summarized in Table 5) were also conjugated to Ab1 and the resulting DARs achieved are summarized in Table 4. The ADCs were synthesized with high and intermediate DAR levels (>7 and ˜4, respectively). Additionally, two different linker systems were synthesized and characterized for potency and stability.

TABLE 4
Average DAR for ADC constructs

| ID | DAR |
|---|---|
| ADC-A | 4 |
| ADC-U | 4.2 |
| ADC-V | 7.1 |
| ADC-W | 3.9 |
| ADC-X | 6.7 |

### Example 2: Potency of Immunoconjugates In Vitro

The ADC-A, ADC-U, ADC-V, ADC-W and ADC-X immunoconjugates, which have different cytotoxic moieties, linker chemistry and DAR were analyzed as described below. Summarily, the potencies of the various ADCs were tested in cell culture using different cell lines, including Mantle cell lymphoma lines JeKo-1 and Mino; triple negative breast cancer cell line MDA-MB-468, and the Mec cell line, which is derived from B-chronic lymphocytic leukemia in prolymphocytic transformation. The Mec cell line was transfected with either an expression vector encoding human ROR1 or a control vector, and stable cell lines were created using selection media containing G418.

Cells were cultured in log phase growth and distributed into 96-well plates. Each cell line was plated at a slightly different cell density, ranging from 5×10³ to 50×10⁴ cells/well. Cells in duplicate were incubated with 3-fold serial dilution of a particular immunoconjugate (10, 3.33, 1.11, 0.37, 0.12, 0.041, 0.014, and 0.0045 μM) for 72 hours at 37° C. and 5% CO₂. After treatment, cells were incubated with an equal volume of CellTiter-Glo® reagent (Promega Inc.) for 15 minutes at room temperature and viability was determined by a luminometer. Curves and EC₅₀ values were generated in GraphPad Prism using a sigmoidal dose response non-linear regression fit. The data from these experiments are summarized in Table 5.

TABLE 5
Cytotoxicity of Exemplary Immunoconjugates in vitro

| | EC₅₀ (nM) | | | | |
|---|---|---|---|---|---|
| | ADC-A | ADC-U | ADC-V | ADC-W | ADC-X |
| Cell line | (DAR 4) | (DAR 4.2) | (DAR 3.9) | (DAR 7.1) | (DAR 6.7) |
| Mec-vector | 558 | >1000 | >1000 | >1000 | >1000 |
| (ROR1 | | | | | |
| negative) | | | | | |
| Mec-ROR1 | 208 | >1000 | 614 | 763 | 256 |
| JeKo-1 | 22-40** | 216 | 88 | 119 | 40 |
| Mino | 19-28** | 156 | 65 | 90 | 36 |
| MDA-MB- | 21-24** | 325 | 145 | 177 | 72 |
| 468 | | | | | |

**96 h incubation, as opposed to 72 h.

The cytotoxicity of all the ADCs was highly dependent on ROR1 as little to no killing of non-transfected Mec cells was observed. The exatecan-containing ADCs (ADC-U, -V, -W, and -X) were all constructed with cleavable linkers. In general, the exatecan-containing ADCs displayed similar potency to ADC-A, which contained an MMAE payload. For the exatecan derivatives (ADC-U, -V, -W, and -X), the higher DAR species displayed greater potency than the corresponding low DAR species (Table 3, compare ADC-V vs. -U and ADC-X vs -W). Moreover, the exatecan ADCs with the phenyl-stabilized linkers (ADC-V and ADC-X) displayed greater potency than the corresponding ADCs lacking the phenyl-stabilized linker (ADC-U and ADC-W, respectively).

### Example 3: Plasma Stability of Immunoconjugates In Vitro

The stability of ADC-U, ADC-V, ADC-W and ADC-X was characterized in the plasma of humans (H), cynomolgus monkeys (C), and mice (M). An immunoaffinity approach was used to enrich each of the ADCs from plasma using agarose beads coated with Protein A. Since the ADCs are too large for practical direct quantitative analysis using LC/MS/MS technology, the bound proteins were subjected to on-bead enzymatic hydrolysis with papain, thereby releasing the conjugated toxin/drug. The released toxin was used as a surrogate for the Total ADC concentrations. For each ADC, the mean time 0 peak area ratio was used as the nominal value. For each subsequent timepoint, the data was expressed as percent of time 0.

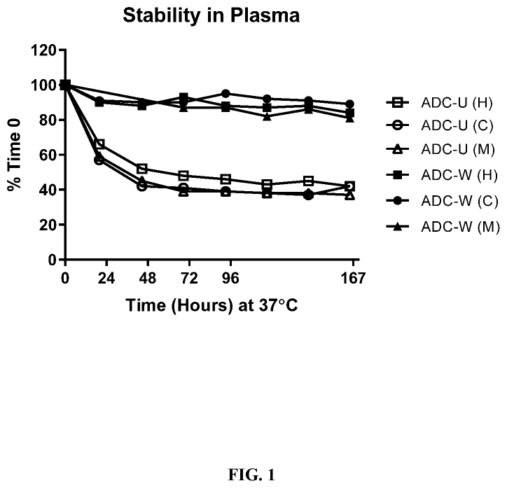

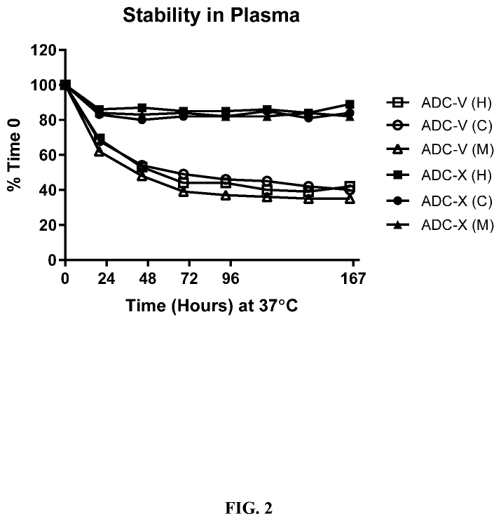

The addition of the phenyl-stabilizing group to the modified linker had a significant impact on the stability of the intact ADC in the plasma of all three species (FIGS. 1 and 2). For example, after incubation in human plasma at 37° C. for ˜7 days (165 h), greater than 84% of the phenyl-stabilized ADCs, ADC-W and ADC-X, remained intact while only 42% of the non-stabilized ADCs, ADC-U and ADC-V remained.

### Example 4: Efficacy of ADC-U and ADC-W in a JeKo-1 Human MCL Xenograft Mouse Model

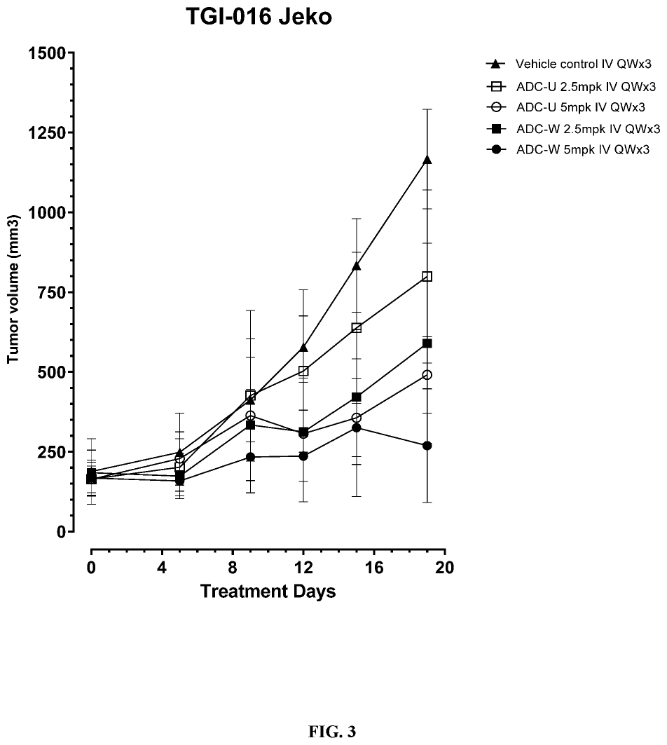

Jeko-1 (MCL) cells were engrafted subcutaneously into mice. When the tumor size reached 200 mm³, the mice were randomized into 5 groups, each group containing 5 mice. The groups were vehicle control, 2.5- and 5 mg/kg ADC-U (DAR 4.2), and 2.5- and 5 mg/kg ADC-W (DAR 3.9). The mice were dosed intravenously (IV) weekly for three weeks. The results of this study are shown in FIG. 3.

All treatments slowed tumor progression compared to vehicle control (closed triangles) and the 5 mg/kg doses (circles) were more efficacious than the 2.5 mg/kg doses (squares). In addition, ADC-W, the ADC with the more stable linker (closed circles and squares) was more efficacious than an equivalent dose of ADC-U (open circle and squares).

### Example 5: Efficacy of ADC-V and ADC-X in a JeKo-1 Human MCL Xenograft Mouse Model

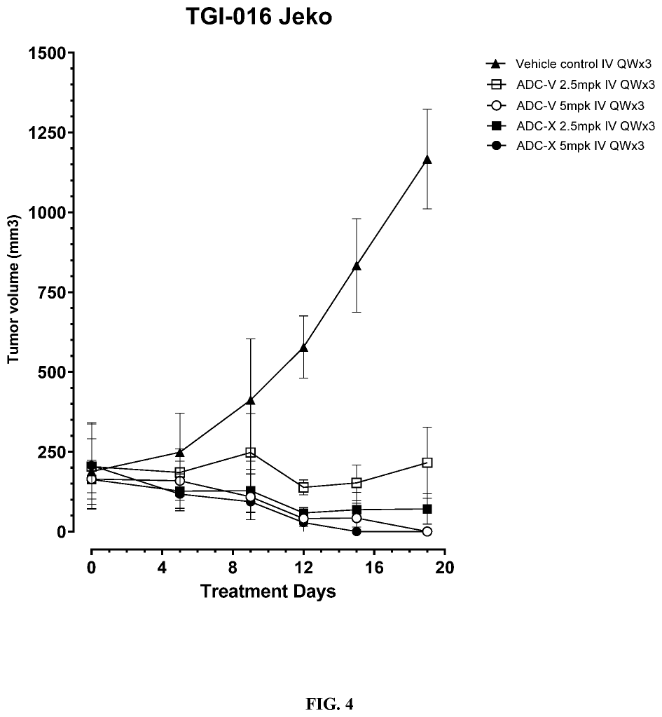

Jeko-1 (MCL) cells were engrafted subcutaneously into mice. When the tumor size reached 200 mm³, the mice were randomized into five groups, 5 mice/group. The groups were vehicle control, 2.5- and 5 mg/kg ADC-V (DAR 7.1), and 2.5- and 5 mg/kg ADC-X (DAR 6.7). The mice were dosed intravenously (IV) weekly for three weeks. The results of this study are shown in FIG. 4. All treatments caused tumor regression compared to vehicle control (closed triangles) and the 5 mg/kg doses (circles) were more efficacious than 2.5 mg/kg doses (squares). In addition, ADC-X, the ADC with the more stable linker (closed squares) was more efficacious than ADC-V at the 2.5 mg/kg doses (open squares).

Collectively, the in vivo studies demonstrated that the constructs with higher DAR (ADC-V and ADC-X) were more potent than the corresponding lower DAR constructs (ADC-U and ADC-W). Additionally, the constructs with stabilized linkers displayed equivalent or superior efficacy to the corresponding constructs that did not contain the stabilized linker.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

### LIST OF SEQUENCES

The amino acid sequences and nucleotide sequences described herein are listed in Table 6 below (SEQ: SEQ ID NO).

TABLE 6
List of Sequences

| SEQ | Description | SEQUENCE |
|---|---|---|
| 1 | Human ROR1 | VATNGKEVVS STGVLFVKFG |
| | fragment | PC |
| 2 | Human ROR1 | EVVSSTGVLF VKFGPC |
| | fragment | |
| 3 | Ab1 heavy | QVQLQESGPGLVKPSQTLSL |
| | chain | TCTVSGYAFTAYNIHWVRQA |
| | | PGQGLEWMGSFDPYDGGSSY |
| | | NQKFKDRLTISKDTSKNQVV |
| | | LTMTNMDPVDTATYYCARGW |
| | | YYFDYWGHGTLVTVSSASTK |
| | | GPSVFPLAPSSKSTSGGTAA |
| | | LGCLVKDYFPEPVTVSWNSG |
| | | ALTSGVHTFPAVLQSSGLYS |
| | | LSSVVTVPSSSLGTQTYICN |
| | | VNHKPSNTKVDKKVEPKSCD |
| | | KTHTCPPCPAPELLGGPSVF |
| | | LFPPKPKDTLMISRTPEVTC |
| | | VVVDVSHEDPEVKFNWYVDG |
| | | VEVHNAKTKPREEQYNSTYR |
| | | VVSVLTVLHQDWLNGKEYKC |
| | | KVSNKALPAPIEKTISKAKG |
| | | QPREPQVYTLPPSRDELTKN |
| | | QVSLTCLVKGFYPSDIAVEW |
| | | ESNGQPENNYKTTPPVLDSD |
| | | GSFFLYSKLTVDKSRWQQGN |
| | | VFSCSVMHEALHNHYTQKSL |
| | | SLSPGK |
| 4 | Ab1 light | DIVMTQTPLSLPVTPGEPAS |
| | chain | ISCRASKSISKYLAWYQQKP |
| | | GQAPRLLIYSGSTLQSGIPP |
| | | RFSGSGYGTDFTLTINNIES |
| | | EDAAYYFCQQHDESPYTFGE |
| | | GTKVEIKRTVAAPSVFIFPP |
| | | SDEQLKSGTASVVCLLNNFY |
| | | PREAKVQWKVDNALQSGNSQ |
| | | ESVTEQDSKDSTYSLSSTLT |
| | | LSKADYEKHKVYACEVTHQG |
| | | LSSPVTKSFNRGEC |
| 5 | Ab1 VH | QVQLQESGPGLVKPSQTLSL |
| | | TCTVSGYAFTAYNIHWVRQA |
| | | PGQGLEWMGSFDPYDGGSSY |
| | | NQKFKDRLTISKDTSKNQVV |
| | | LTMTNMDPVDTATYYCARGW |
| | | YYFDYWGHGTLVTVSS |
| 6 | Ab1 VL | DIVMTQTPLSLPVTPGEPAS |
| | | ISCRASKSISKYLAWYQQKP |
| | | GQAPRLLIYSGSTLQSGIPP |
| | | RFSGSGYGTDFTLTINNIES |
| | | EDAAYYFCQQHDESPYTFGE |
| | | GTKVEIK |
| 7 | Ab1 HCDR1 | GYAFTAYN |
| 8 | Ab1 HCDR2 | FDPYDGGS |
| 9 | Ab1 HCDR3 | GWYYFDY |
| 10 | Ab1 LCDR1 | KSISKY |
| 11 | Ab1 LCDR2 | SGS |
| 12 | Ab1 LCDR3 | QQHDESPY |
| 13 | Ab1 VH frag. | SGYAFTAYNIHWVRQ |
| 14 | Ab1 VH frag. | GSFDPYDGGSSYNQKF |
| 15 | Ab1 VH frag. | YYCARGWYYFDYWGHGTLVT |
| | | VSS |
| 16 | Ab1 VL frag. | CRASKSISKYLAWY |
| 17 | Ab1 VL frag. | LLIYSGSTLQSG |
| 18 | Ab1 VL frag. | CQQHDESPYTFGEGTKVEIK |
| 19 | Ab1 heavy | AAGCTTACCGCCACCATGGG |
| | chain coding | CTGGAGCTGTATCATCCTCT |
| | sequence | TCCTGGTGGCGACCGCGACG |
| | | GGTGTCCACTCCCAGGTGCA |
| | | GCTCCAGGAGTCCGGCCCCG |
| | | GGCTTGTGAAGCCGTCACAA |
| | | ACCCTGTCCCTGACGTGCAC |
| | | GGTCTCCGGCTACGCCTTCA |
| | | CGGCCTACAACATACATTGG |
| | | GTCCGGCAGGCGCCGGGCCA |
| | | GG |
| | | GGCTGGAGTGGATGGGTTCC |
| | | TTCGACCCGTACGATGGCGG |
| | | GAGCTCGTACAACCAGAAGT |
| | | TCAAAGACCGCCTGACGATC |
| | | TCCAAGGACACCTCGAAAAA |
| | | CCAGGTCGTCCTGACCATGA |
| | | CCAACATGGACCCGGTGGAC |
| | | ACGGCGACCTACTATTGCGC |
| | | CCGCGGCTGGTACTACTTCG |
| | | ACTACTGGGGCCACGGGACC |
| | | CTGGTCACCGTGTCTTCCGC |
| | | TTCGACCAAGGGCCCCAGCG |
| | | TCTTCCCGCTCGCGCCCTCC |
| | | TCGAAGTCCACCTCGGGCGG |
| | | CACTGCCGCCCTGGGCTGCC |
| | | TGGTCAAGGACTACTTCCCG |
| | | GAGCCGGTGACCGTCTCGTG |
| | | GAACAGCGGGGCACTCACCT |
| | | CCGGCGTGCACACCTTCCCG |
| | | GCCGTGCTGCAGTCCTCGGG |
| | | GCTGTATTCACTCAGCTCGG |
| | | TCGTCACCGTCCCCTCGTCG |
| | | TCCCTCGGCACGCAGACGTA |
| | | CATCTGCAACGTCAACCACA |
| | | AGCCCTCGAACACCAAGGTG |
| | | GACAAGAAGGTCGAGCCGAA |
| | | GTCCTGCGATAAGACCCACA |
| | | CCTGCCCCCCGTGCCCGGCC |
| | | CCCGAGCTCCTGGGCGGTCC |
| | | GTCCGTGTTCCTCTTCCCGC |
| | | CCAAGCCCAAGGACACCCTG |
| | | ATGATCAGCCGCACGCCCGA |
| | | GGTGACCTGCGTCGTCGTGG |
| | | ACGTCTCCCACGAGGATCCC |
| | | GAGGTGAAGTTCAACTGGTA |
| | | CGTGGACGGGGTGGAGGTCC |
| | | ACAACGCCAAGACAAAGCCG |
| | | CGGGAAGAGCAGTACAACTC |
| | | GACCTACCGCGTCGTCAGCG |
| | | TGCTGACGGTCCTCCACCAG |
| | | GACTGGCTGAACGGCAAGGA |
| | | GTACAAATGCAAGGTGTCCA |
| | | ACAAGGCCCTGCCCGCGCCC |
| | | ATCGAGAAGACCATCTCCAA |
| | | GGCCAAGGGACAGCCGCGCG |
| | | AGCCGCAGGTCTACACGCTG |
| | | CCTCCCTCCCGGGACGAGCT |
| | | CACGAAGAACCAGGTATCGC |
| | | TCACCTGCCTCGTGAAGGGC |
| | | TTCTACCCGAGCGACATCGC |
| | | CGTCGAGTGGGAGAGCAACG |
| | | GCCAGCCCGAGAACAACTAC |
| | | AAAACCACGCCGCCGGTCCT |
| | | CGACTCTGACGGGTCCTTCT |
| | | TCCTGTACTCCAAGCTGACC |
| | | GTGGACAAGTCGCGGTGGCA |
| | | GCAGGGGAACGTGTTCTCGT |
| | | GCTCGGTCATGCACGAGGCG |
| | | TTGCACAACCACTACACCCA |
| | | GAAGTCACTCTCCCTGAGCC |
| | | CGGGCAAGTGATAATCTAGA |
| | | GTCGGGGCGGCCGGCC |
| 20 | Ab1 light | AAGCTTACCGCCACCATGGG |
| | chain coding | CTGGTCATGCATCATCCTGT |
| | sequence | TCCTGGTCGCCACCGCGACG |
| | | GGGGTCCACAGTGATATCGT |
| | | CATGACGCAGACGCCGCTGA |
| | | GCCTCCCGGTGACGCCCGGC |
| | | GAGCCCGCCAGCATCTCCTG |
| | | CCGCGCTTCCAAGTCCATCT |
| | | CGAAGTACCTGGCGTGGTAT |
| | | CAGCAGAAGCCCGGCCAGGC |
| | | CCCGCGCCTGCTCATCTACT |
| | | CTGGTTCCACGCTCCAGTCG |
| | | GGCATCCCGCCCCGGTTCTC |
| | | GGGTTCGGGATACGGCACCG |
| | | ACTTCACCCTGACCATCAAC |
| | | AACATCGAGAGCGAAGACGC |
| | | GGCGTACTACTTCTGCCAGC |
| | | AGCACGACGAGTCCCCGTAC |
| | | ACCTTCGGCGAGGGGACCAA |
| | | GGTCGAGATCAAGCGTACCG |
| | | TCGCGGCACCGAGCGTCTTC |
| | | ATCTTCCCCCCGTCCGACGA |
| | | GCAGCTCAAGTCTGGCACCG |
| | | CCTCGGTCGTCTGTCTCCTG |
| | | AACAACTTCTACCCCAGGGA |
| | | AGCCAAGGTCCAGTGGAAGG |
| | | TGGACAACGCGCTGCAGTCC |
| | | GGGAATAGCCAGGAGTCGGT |
| | | GACGGAGCAGGACTCCAAGG |
| | | ACTCCACGTACTCGCTCTCG |
| | | TCCACCCTGACCCTCTCCAA |
| | | GGCGGACTACGAAAAGCACA |
| | | AGGTCTACGCCTGCGAGGTG |
| | | ACGCACCAAGGCCTGTCCTC |
| | | CCCAGTGACCAAGTCGTTCA |
| | | ACCGCGGCGAGTGCTGATAA |
| | | TCTAGAGTCGGGGCGGCCGG |
| | | CC |
| 21 | Ab1 VH | AAGCTTACCGCCACCATGGG |
| | coding | CTGGAGCTGTATCATCCTCT |
| | sequence l | TCCTGGTGGCGACCGCGACG |
| | | GGTGTCCACTCCCAGGTGCA |
| | | GCTCCAGGAGTCCGGCCCCG |
| | | GGCTTGTGAAGCCGTCACAA |
| | | ACCCTGTCCCTGACGTGCAC |
| | | GGTCTCCGGCTACGCCTTCA |
| | | CGGCCTACAACATACATTGG |
| | | GTCCGGCAGGCGCCGGGCCA |
| | | GGGGCTGGAGTGGATGGGTT |
| | | CCTTCGACCCGTACGATGGC |
| | | GGGAGCTCGTACAACCAGAA |
| | | GTTCAAAGACCGCCTGACGA |
| | | TCTCCAAGGACACCTCGAAA |
| | | AACCAGGTCGTCCTGACCAT |
| | | GACCAACATGGACCCGGTGG |
| | | ACACGGCGACCTACTATTGC |
| | | GCCCGCGGCTGGTACTACTT |
| | | CGACTACTGGGGCCACGGGA |
| | | CCCTGGTCACCGTGTCTTCC |
| 22 | Ab1 VL | GATATCGTCATGACGCAGAC |
| | coding | GCCGCTGAGCCTCCCGGTGA |
| | sequence 1 | CGCCCGGCGAGCCCGCCAGC |
| | | ATCTCCTGCCGCGCTTCCAA |
| | | GTCCATCTCGAAGTACCTGG |
| | | CGTGGTATCAGCAGAAGCCC |
| | | GGCCAGGCCCCGCGCCTGCT |
| | | CATCTACTCTGGTTCCACGC |
| | | TCCAGTCGGGCATCCCGCCC |
| | | CGGTTCTCGGGTTCGGGATA |
| | | CGGCACCGACTTCACCCTGA |
| | | CCATCAACAACATCGAGAGC |
| | | GAAGACGCGGCGTACTACTT |
| | | CTGCCAGCAGCACGACGAGT |
| | | CCCCGTACACCTTCGGCGAG |
| | | GGGACCAAGGTCGAGATCAA |
| | | G |
| 23 | Abl VH | CAGGTGCAGCTGCAGGAGTC |
| | coding | GGGCCCAGGACTGGTGAAGC |
| | sequence 2 | CTTCACAGACCCTGTCCCTC |
| | | ACCTGCACTGTCTCTGGTTA |
| | | TGCATTCACTGCCTACAACA |
| | | TACACTGGGTGCGACAGGCC |
| | | CCTGGACAAGGGCTTGAGTG |
| | | GATGGGTTCTTTTGATCCTT |
| | | ACGATGGTGGTAGTAGTTAC |
| | | AACCAGAAGTTCAAGGACAG |
| | | ACTCACCATCTCCAAGGACA |
| | | CCTCCAAAAACCAGGTGGTC |
| | | CTTACAATGACCAACATGGA |
| | | CCCTGTGGACACAGCCACGT |
| | | ATTACTGTGCAAGAGGGTGG |
| | | TACTACTTTGACTACTGGGG |
| | | CCACGGAACCCTGGTCACCG |
| | | TCTCCTCA |
| 24 | Ab1 VL | GATATTGTGATGACCCAGAC |
| | coding | TCCACTCTCCCTGCCCGTCA |
| | sequence 2 | CCCCTGGAGAGCCGGCCTCC |
| | | ATCTCCTGCAGGGCAAGTAA |
| | | GAGCATTAGCAAATATTTAG |
| | | CCTGGTACCAGCAGAAACCT |
| | | GGCCAGGCTCCCAGGCTCCT |
| | | CATCTATTCTGGATCCACTT |
| | | TGCAATCTGGGATCCCACCT |
| | | CGATTCAGTGGCAGCGGGTA |
| | | TGGAACAGATTTTACCCTCA |
| | | CAATTAATAACATAGAATCT |
| | | GAGGATGCTGCATATTACTT |
| | | CTGTCAACAGCATGATGAAT |
| | | CCCCGTACACGTTCGGCGAG |
| | | GGGACCAAGGTGGAAATCAA |
| | | A |
| 25 | D10 VH | QVOLKESGPGLVAPSQTLSI |
| | | TCTVSGFSLTSYGVHWVRQP |
| | | PGKGLEWLGVIWAGGFTNYN |
| | | SALKSRLSISKDNSKSQVLL |
| | | KMTSLQTDDTAMYYCARRGS |
| | | SYSMDYWGQGTSVIVSS |
| 26 | D10 VL | EIVLSQSPAITAASLGQKVT |
| | | ITCSASSNVSYIHWYQQRSG |
| | | TSPRPWIYEISKLASGVPVR |
| | | FSGSGSGTSYSLTISSMEAE |
| | | DAAIYYCQQWNYPLITFGSG |
| | | TKLEIQ |
| 27 | D10 HCDR1 | GFSLTSYG |
| 28 | D10 HCDR2 | WAGGFT |
| 29 | D10 HCDR3 | RGSSYSMDY |
| 30 | D10 LCDR1 | SNVSYI |
| 31 | D10 LCDR2 | EIS |
| 32 | D10 LCDR3 | QQWNYPLI |
| 33 | D10 VH | CAGGTGCAGCTGAAGGAGTC |
| | coding | AGGACCTGGCCTGGTGGCGC |
| | sequence | CCTCACAGACTCTGTCCATC |
| | | ACTTGCACTGTCTCTGGGTT |
| | | TTCATTAACCAGTTATGGTG |
| | | TACACTGGGTTCGCCAGCCT |
| | | CCAGGAAAGGGTCTGGAGTG |
| | | GCTGGGAGTAATATGGGCTG |
| | | GTGGATTCACAAATTATAAT |
| | | TCGGCTCTCAAGTCCAGACT |
| | | GAGCATCAGCAAAGACAACT |
| | | CCAAGAGCCAAGTTCTCTTA |
| | | AAAATGACCAGTCTGCAAAC |
| | | TGATGACACAGCCATGTACT |
| | | ACTGTGCCAGGAGAGGTAGT |
| | | TCCTATTCTATGGACTATTG |
| | | GGGTCAAGGAACCTCAGTCA |
| | | CCGTCTCCTCA |
| 34 | D10 VL | GAAATTGTGCTCTCTCAGTC |
| | coding | TCCAGCCATCACAGCTGCAT |
| | sequence | CTCTGGGCCAAAAGGTCACC |
| | | ATCACCTGCAGTGCCAGTTC |
| | | AAATGTAAGTTACATCCACT |
| | | GGTACCAGCAGAGGTCAGGC |
| | | ACCTCCCCCAGACCATGGAT |
| | | TTATGAAATATCCAAACTGG |
| | | CTTCTGGAGTCCCAGTTCGC |
| | | TTCAGTGGCAGTGGGTCTGG |
| | | GACCTCTTACTCTCTCACAA |
| | | TCAGCAGCATGGAGGCTGAA |
| | | GATGCTGCCATTTATTATTG |
| | | TCAGCAGTGGAATTATCCTC |
| | | TTATCACGTTCGGCTCGGGG |
| | | ACAAAGTTGGAAATACAA |
| 35 | 4A5 VH | EVKLVESGGGLVKPGGSLKL |
| | | SCAASGFTFSSYAMSWVRQI |
| | | PEKRLEWVASISRGGTTYYP |
| | | DSVKGRFTISRDNVRNILYL |
| | | QMSSLRSEDTAMYYCGRYDY |
| | | DGYYAMDYWGQGTSVTVSS |
| 36 | 4A5 VL | DIKMTOSPSSMYASLGERVT |
| | | ITCKASPDINSYLSWFQQKP |
| | | GKSPKTLIYRANRLVDGVPS |
| | | RFSGGGSGODYSLTINSLEY |
| | | EDMGIYYCLQYDEFPYTFGG |
| | | GTKLEMK |
| 37 | 4A5 HCDR1 | GFTFSSYA |
| 38 | 4A5 HCDR2 | ISRGGTT |
| 39 | 4A5 HCDR3 | YDYDGYYAMDY |
| 40 | 4A5 LCDR1 | PDINSY |
| 41 | 4A5 LCDR2 | RAN |
| 42 | 4AS LCDR3 | LQYDEFPYT |
| 43 | 4A5 VH | GAAGTGAAACTGGTGGAGTC |
| | coding | TGGGGGAGGCTTAGTGAAGC |
| | sequence | CTGGAGGGTCCCTGAAACTC |
| | | TCCTGTGCAGCCTCTGGATT |
| | | CACTTTCAGTAGCTATGCCA |
| | | TGTCTTGGGTTCGCCAGATT |
| | | CCAGAGAAGAGGCTGGAGTG |
| | | GGTCGCATCCATTAGTCGTG |
| | | GTGGTACCACCTACTATCCA |
| | | GACAGTGTGAAGGGCCGATT |
| | | CACCATCTCCAGAGATAATG |
| | | TCAGGAACATCCTGTACCTG |
| | | CAAATGAGCAGTCTGAGGTC |
| | | TGAGGACACGGCCATGTATT |
| | | ACTGTGGAAGATATGATTAC |
| | | GACGGGTACTATGCAATGGA |
| | | CTACTGGGGTCAAGGAACCT |
| | | CAGTCACCGTCTCCTCA |
| 44 | 4A5 VL | GACATCAAGATGACCCAGTC |
| | coding | TCCATCTTCCATGTATGCAT |
| | sequence | CTCTAGGAGAGAGAGTCACT |
| | | ATCACTTGCAAGGCGAGTCC |
| | | GGACATTAATAGCTATTTAA |
| | | GCTGGTTCCAGCAGAAACCA |
| | | GGGAAATCTCCTAAGACCCT |
| | | GATCTATCGTGCAAACAGAT |
| | | TGGTTGATGGGGTCCCATCA |
| | | AGGTTCAGTGGCGGTGGATC |
| | | TGGGCAAGATTATTCTCTCA |
| | | CCATCAACAGCCTGGAGTAT |
| | | GAAGATATGGGAATTTATTA |
| | | TTGTCTACAGTATGATGAAT |
| | | TTCCGTACACGTTCGGAGGG |
| | | GGGACCAAGCTGGAAATGAA |
| | | AC |
| 45 | 99961 VH | EIQLQQSGPVLVKPGASVKV |
| | | SCKASGYAFTAYNIHWVRQS |
| | | HGKRLEWIGSFDPYDGGSSY |
| | | NQKFKDKATLTVDKSSTTAY |
| | | MHLNSLTSEDSAVYYCARGW |
| | | YYFDYWGHGTTLTVSS |
| 46 | 99961 VL | DVQITQSPSYLAASPGETIT |
| | | INCRASKSISKYLAWYQEKP |
| | | GKTNKLLIYSGSTLQSGIPS |
| | | RFRGSGSGTDFTLTISSLEP |
| | | EDFAMYYCQQHDESPYTFGE |
| | | GTKLEIKR |
| 47 | Ab4 heavy | QVQLQESGPGLVKPSQTLSL |
| | chain | TCTVSGYAFTAYNIHWIRQP |
| | | PGKGLEWIGSFDPYDGGSSY |
| | | NQKFKDRLTISKDTSKNQVV |
| | | LTMTNMDPVDTATYYCARGW |
| | | YYFDYWGHGTLVTVSSASTK |
| | | GPSVFPLAPSSKSTSGGTAA |
| | | LGCLVKDYFPEPVTVSWNSG |
| | | ALTSGVHTFPAVLQSSGLYS |
| | | LSSVVTVPSSSLGTQTYICN |
| | | VNHKPSNTKVDKKVEPKSCD |
| | | KTHTCPPCPAPELLGGPSVF |
| | | LFPPKPKDTLMISRTPEVTC |
| | | VVVDVSHEDPEVKFNWYVDG |
| | | VEVHNAKTKPREEQYNSTYR |
| | | VVSVLTVLHQDWLNGKEYKC |
| | | KVSNKALPAPIEKTISKAKG |
| | | QPREPQVYTLPPSRDELTKN |
| | | QVSLTCLVKGFYPSDIAVEW |
| | | ESNGQPENNYKTTPPVLDSD |
| | | GSFFLYSKLTVDKSRWQQGN |
| | | VFSCSVMHEALHNHYTQKSL |
| | | SLSPGK |
| 48 | Ab4 VH | QVQLQESGPGLVKPSQTLSL |
| | | TCTVSGYAFTAYNIHWIRQP |
| | | PGKGLEWIGSFDPYDGGSSY |
| | | NQKFKDRLTISKDTSKNQVV |
| | | LTMTNMDPVDTATYYCARGW |
| | | YYFDYWGHGTLVTVSS |
| 49 | Ab4 light | DVVMTQSPLSLPVTLGQPAS |
| | chain | ISCRASKSISKYLAWYQQKP |
| | | GKAPKLLIYSGSTLQSGIPP |
| | | RFSGSGYGTDFTLTINNIES |
| | | EDAAYYFCQQHDESPYTFGE |
| | | GTKVEIKRTVAAPSVFIFPP |
| | | SDEQLKSGTASVVCLLNNFY |
| | | PREAKVQWKVDNALQSGNSQ |
| | | ESVTEQDSKDSTYSLSSTLT |
| | | LSKADYEKHKVYACEVTHQG |
| | | LSSPVTKSFNRGEC |
| 50 | Ab4 VL | DVVMTQSPLSLPVTLGQPAS |
| | | ISCRASKSISKYLAWYQQKP |
| | | GKAPKLLIYSGSTLQSGIPP |
| | | RFSGSGYGTDFTLTINNIES |
| | | EDAAYYFCQQHDESPYTFGE |
| | | GTKVEIK |
| 51 | Ab4 heavy | CAGGTCCAGCTGCAGGAGTC |
| | chain coding | AGGTCCCGGACTGGTCAAGC |
| | sequence | CGTCGCAGACGCTGTCCCTC |
| | | ACCTGCACCGTGTCGGGCTA |
| | | CGCCTTCACCGCCTACAACA |
| | | TCCACTGGATCCGTCAGCCC |
| | | CCTGGGAAGGGCCTCGAGTG |
| | | GATCGGCAGCTTCGACCCGT |
| | | ACGACGGCGGGTCGTCCTAC |
| | | AACCAGAAGTTCAAGGACCG |
| | | CCTCACCATCAGCAAGGACA |
| | | CCTCCAAGAACCAGGTCGTC |
| | | CTCACCATGACCAACATGGA |
| | | CCCCGTGGACACCGCCACGT |
| | | ACTACTGCGCGCGGGGCTGG |
| | | TACTACTTCGACTACTGGGG |
| | | GCACGGCACCCTCGTCACGG |
| | | TCTCGTCGGCGAGCACCAAG |
| | | GGTCCGAGCGTCTTCCCCCT |
| | | GGCCCCCTCCAGCAAGTCCA |
| | | CCTCGGGGGGCACCGCCGCC |
| | | CTGGGCTGCCTGGTCAAGGA |
| | | CTACTTCCCCGAGCCCGTGA |
| | | CCGTGAGCTGGAACTCCGGC |
| | | GCCCTCACCAGCGGGGTCCA |
| | | CACCTTCCCGGCGGTCCTGC |
| | | AGTCATCCGGTCTCTACTCC |
| | | TTGAGCTCAGTCGTCACCGT |
| | | CCCGAGCTCCTCCCTCGGAA |
| | | CGCAGACCTACATCTGCAAC |
| | | GTCAACCACAAGCCGTCCAA |
| | | CACCAAGGTCGACAAGAAGG |
| | | TGGAGCCCAAATCGTGCGAC |
| | | AAGACCCACACCTGCCCGCC |
| | | GTGCCCCGCCCCGGAACTGC |
| | | TCGGCGGCCCCTCGGTGTTC |
| | | CTGTTCCCCCCGAAGCCCAA |
| | | GGACACCCTCATGATCTCCC |
| | | GCACCCCCGAGGTCACCTGC |
| | | GTGGTGGTGGATGTCTCCCA |
| | | CGAGGACCCCGAGGTGAAGT |
| | | TCAACTGGTACGTGGACGGG |
| | | GTCGAGGTGCACAACGCCAA |
| | | GACCAAGCCCCGAGAGGAAC |
| | | AGTATAACTCGACGTACCGC |
| | | GTGGTCAGCGTCCTGACCGT |
| | | GCTCCACCAGGACTGGCTGA |
| | | ACGGCAAGGAGTACAAGTGC |
| | | AAGGTCAGCAACAAGGCCCT |
| | | GCCCGCCCCCATCGAGAAGA |
| | | CGATCTCCAAGGCGAAGGGG |
| | | CAGCCGCGCGAGCCGCAGGT |
| | | CTACACCCTGCCGCCCAGCC |
| | | GGGACGAGCTCACGAAGAAT |
| | | CAGGTCTCGCTCACCTGCCT |
| | | CGTCAAGGGTTTCTACCCGT |
| | | CGGACATCGCGGTCGAATGG |
| | | GAGTCGAACGGTCAGCCCGA |
| | | GAATAACTACAAGACGACCC |
| | | CGCCCGTCCTGGACTCGGAC |
| | | GGCAGCTTCTTCCTGTACTC |
| | | GAAGCTGACGGTCGACAAGT |
| | | CGCGCTGGCAGCAGGGCAAC |
| | | GTCTTCTCGTGCTCGGTGAT |
| | | GCACGAGGCCCTCCACAACC |
| | | ACTACACACAGAAGAGCCTC |
| | | TCGCTTTCGCCGGGCAAG |
| 52 | Ab4 VH | CAGGTCCAGCTGCAGGAGTC |
| | coding | AGGTCCCGGACTGGTCAAGC |
| | sequence | CGTCGCAGACGCTGTCCCTC |
| | | ACCTGCACCGTGTCGGGCTA |
| | | CGCCTTCACCGCCTACAACA |
| | | TCCACTGGATCCGTCAGCCC |
| | | CCTGGGAAGGGCCTCGAGTG |
| | | GATCGGCAGCTTCGACCCGT |
| | | ACGACGGCGGGTCGTCCTAC |
| | | AACCAGAAGTTCAAGGACCG |
| | | CCTCACCATCAGCAAGGACA |
| | | CCTCCAAGAACCAGGTCGTC |
| | | CTCACCATGACCAACATGGA |
| | | CCCCGTGGACACCGCCACGT |
| | | ACTACTGCGCGCGGGGCTGG |
| | | TACTACTTCGACTACTGGGG |
| | | GCACGGCACCCTCGTCACGG |
| | | TCTCGTCG |
| 53 | Ab4 light | GACGTCGTGATGACCCAGTC |
| | chain coding | GCCCCTCTCCCTGCCGGTTA |
| | sequence | CCCTGGGCCAGCCCGCCTCC |
| | | ATCAGCTGCCGTGCCTCCAA |
| | | GTCCATTTCCAAGTACCTGG |
| | | CCTGGTACCAGCAGAAGCCG |
| | | GGGAAGGCCCCAAAGCTCCT |
| | | CATCTACTCCGGCTCCACCC |
| | | TCCAGAGCGGCATCCCCCCC |
| | | CGCTTCAGCGGCTCCGGCTA |
| | | CGGCACCGACTTCACCCTCA |
| | | CCATCAATAACATCGAGTCG |
| | | GAGGACGCCGCGTACTACTT |
| | | CTGCCAGCAGCACGACGAAT |
| | | CGCCGTACACCTTCGGGGAG |
| | | GGCACCAAGGTGGAGATCAA |
| | | GAGGACGGTCGCCGCGCCCT |
| | | CCGTGTTCATCTTCCCCCCC |
| | | TCGGACGAACAGCTGAAGTC |
| | | CGGGACCGCCTCCGTCGTCT |
| | | GCCTCCTCAACAACTTCTAC |
| | | CCGCGCGAGGCCAAGGTGCA |
| | | GTGGAAGGTCGACAACGCGC |
| | | TCCAGTCCGGCAACTCCCAG |
| | | GAGTCGGTGACCGAGCAGGA |
| | | CTCGAAGGACAGTACCTACT |
| | | CGCTGAGCTCCACACTGACG |
| | | CTCTCGAAGGCCGACTACGA |
| | | GAAGCACAAGGTGTACGCAT |
| | | GCGAGGTGACCCACCAGGGG |
| | | CTGAGCTCGCCGGTGACTAA |
| | | GTCGTTCAACAGGGGCGAAT |
| | | GC |
| 54 | Ab4 VL | GACGTCGTGATGACCCAGTC |
| | coding | GCCCCTCTCCCTGCCGGTTA |
| | sequence | CCCTGGGCCAGCCCGCCTCC |
| | | ATCAGCTGCCGTGCCTCCAA |
| | | GTCCATTTCCAAGTACCTGG |
| | | CCTGGTACCAGCAGAAGCCG |
| | | GGGAAGGCCCCAAAGCTCCT |
| | | CATCTACTCCGGCTCCACCC |
| | | TCCAGAGCGGCATCCCCCCC |
| | | CGCTTCAGCGGCTCCGGCTA |
| | | CGGCACCGACTTCACCCTCA |
| | | CCATCAATAACATCGAGTCG |
| | | GAGGACGCCGCGTACTACTT |
| | | CTGCCAGCAGCACGACGAAT |
| | | CGCCGTACACCTTCGGGGAG |
| | | GGCACCAAGGTGGAGATCAA |
| | | G |
| 55 | Linker peptide | GGFG |
| | moiety | |
| | sequence | |
| 56 | Linker peptide | ALAL |
| | moiety | |
| | sequence | |
| 57 | Linker peptide | GFLG |
| | moiety | |
| | sequence | |
| 58 | Human CH₁ | ASTKGPSVFPLAPSSKSTSG |
| | domain plus | GTAALGCLVKDYFPEPVTVS |
| | upper hinge | WNSGALTSGVHTFPAVLQSS |
| | region | GLYSLSSVVTVPSSSLGTQT |
| | | YICNVNHKPSNTKVDKKVEP |
| | | KSC |
| 50 | Human Kappa | RTVAAPSVFIFPPSDEQLKS |
| | constant | GTASVVCLLNNFYPREAKVQ |
| | domain | WKVDNALQSGNSQESVTEQD |
| | | SKDSTYSLSSTLTLSKADYE |
| | | KHKVYACEVTHQGLSSPVTK |
| | | SFNRGEC |
| 60 | Humanized | DIQMTQSPSSLSASVGDRVT |
| | 4A5 scFv | ITCKASPDINSYLSWFQQRP |
| | | GQSPRRLIYRANRLVDGVPD |
| | | RFSGSGSGTDFTLKISRVEA |
| | | EDVGVYYCLQYDEFPYTFGQ |
| | | GTKVEIKGGGGSGSTSGSGK |
| | | PGSGEGSTKGGGGGSEVQLV |
| | | QSGAEVKKPGESLRISCKGS |
| | | GFTFSSYAMSWIRQSPSRGL |
| | | EWLGSISRGGTTYYPDSVKG |
| | | RFTISRDNAKNSLYLQMNSL |
| | | RAEDTAVYYCGRYDYDGYYA |
| | | MDYWGQGTLVTVSS |
| 61 | Ab1 scFv | DIVMTQTPLSLPVTPGEPAS |
| | | ISCRASKSISKYLAWYQQKP |
| | | GQAPRLLIYSGSTLQSGIPP |
| | | RFSGSGYGTDFTLTINNIES |
| | | EDAAYYFCQQHDESPYTFGE |
| | | GTKVEIKGGGGSGSTSGSGK |
| | | PGSGEGSTKGGGGGSQVQLQ |
| | | ESGPGLVKPSQTLSLTCTVS |
| | | GYAFTAYNIHWVRQAPGQGL |
| | | EWMGSFDPYDGGSSYNQKFK |
| | | DRLTISKDTSKNQVVLTMTN |
| | | MDPVDTATYYCARGWYYFDY |
| | | WGHGTLVTVSS |
| 62 | Ab2 scFv | DVVMTQSPLSLPVTLGQPAS |
| | | ISCRASKSISKYLAWYQQKP |
| | | GKAPKLLIYSGSTLQSGIPP |
| | | RFSGSGYGTDFTLTINNIES |
| | | EDAAYYFCQQHDESPYTFGE |
| | | GTKVEIKGGGGSGSTSGSGK |
| | | PGSGEGSTKGGGGGSQVQLQ |
| | | ESGPGLVKPSQTLSLTCTVS |
| | | GYAFTAYNIHWVRQAPGQGL |
| | | EWMGSFDPYDGGSSYNQKFK |
| | | DRLTISKDTSKNQVVLTMTN |
| | | MDPVDTATYYCARGWYYFDY |
| | | WGHGTLVTVSS |
| 63 | Ab3 scFv | DIVMTQTPLSLPVTPGEPAS |
| | | ISCRASKSISKYLAWYQQKP |
| | | GQAPRLLIYSGSTLQSGIPP |
| | | RFSGSGYGTDFTLTINNIES |
| | | EDAAYYFCQQHDESPYTFGE |
| | | GTKVEIKGGGGSGSTSGSGK |
| | | PGSGEGSTKGGGGGSQVQLQ |
| | | ESGPGLVKPSQTLSLTCTVS |
| | | GYAFTAYNIHWIRQPPGKGL |
| | | EWIGSFDPYDGGSSYNQKFK |
| | | DRLTISKDTSKNQVVLTMTN |
| | | MDPVDTATYYCARGWYYFDY |
| | | WGHGTLVTVSS |
| 64 | Ab4 scFv | DVVMTQSPLSLPVTLGQPAS |
| | | ISCRASKSISKYLAWYQQKP |
| | | GKAPKLLIYSGSTLQSGIPP |
| | | RFSGSGYGTDFTLTINNIES |
| | | EDAAYYFCQQHDESPYTFGE |
| | | GTKVEIKGGGGSGSTSGSGK |
| | | PGSGEGSTKGGGGGSQVQLQ |
| | | ESGPGLVKPSQTLSLTCTVS |
| | | GYAFTAYNIHWIRQPPGKGL |
| | | EWIGSFDPYDGGSSYNQKFK |
| | | DRLTISKDTSKNQVVLTMTN |
| | | MDPVDTATYYCARGWYYFDY |
| | | WGHGTLVTVSS |
| 65 | Ab5/Ab7 | GYAFAAYNIH |
| | HCDR1 | |
| 66 | Ab6/Ab7 | SGSRLQS |
| | LCDR2 | |
| 67 | Ab5/Ab7 | QVQLQESGPGLVKPSQTLSL |
| | VH | TCTVSGYAFAAYNIHWVRQA |
| | | PGQGLEWMGSFDPYDGGSSY |
| | | NQKFKDRLTISKDTSKNQVV |
| | | LTMTNMDPVDTATYYCARGW |
| | | YYFDYWGHGTLVTVSS |
| 68 | Ab6/Ab7 | DIVMTQTPLSLPVTPGEPAS |
| | VL | ISCRASKSISKYLAWYQQKP |
| | | GQAPRLLIYSGSRLQSGIPP |
| | | RFSGSGYGTDFTLTINNIES |
| | | EDAAYYFCQQHDESPYTFGE |
| | | GTKVEIK |
| 69 | Ab5/Ab7 HC | QVQLQESGPGLVKPSQTLSL |
| | | TCTVSGYAFAAYNIHWVRQA |
| | | PGQGLEWMGSFDPYDGGSSY |
| | | NQKFKDRLTISKDTSKNQVV |
| | | LTMTNMDPVDTATYYCARGW |
| | | YYFDYWGHGTLVTVSSASTK |
| | | GPSVFPLAPSSKSTSGGTAA |
| | | LGCLVKDYFPEPVTVSWNSG |
| | | ALTSGVHTFPAVLQSSGLYS |
| | | LSSVVTVPSSSLGTQTYICN |
| | | VNHKPSNTKVDKKVEPKSCD |
| | | KTHTCPPCPAPELLGGPSVF |
| | | LFPPKPKDTLMISRTPEVTC |
| | | VVVDVSHEDPEVKFNWYVDG |
| | | VEVHNAKTKPREEQYNSTYR |
| | | VVSVLTVLHQDWLNGKEYKC |
| | | KVSNKALPAPIEKTISKAKG |
| | | QPREPQVYTLPPSRDELTKN |
| | | QVSLTCLVKGFYPSDIAVEW |
| | | ESNGQPENNYKTTPPVLDSD |
| | | GSFFLYSKLTVDKSRWQQGN |
| | | VFSCSVMHEALHNHYTQKSL |
| | | SLSPGK |
| 70 | Ab6/Ab7 LC | DIVMTQTPLSLPVTPGEPAS |
| | | ISCRASKSISKYLAWYQQKP |
| | | GQAPRLLIYSGSRLQSGIPP |
| | | RFSGSGYGTDFTLTINNIES |
| | | EDAAYYFCQQHDESPYTFGE |
| | | GTKVEIKRTVAAPSVFIFPP |
| | | SDEQLKSGTASVVCLLNNFY |
| | | PREAKVQWKVDNALQSGNSQ |
| | | ESVTEQDSKDSTYSLSSTLT |
| | | LSKADYEKHKVYACEVTHQG |
| | | LSSPVTKSFNRGEC |

1. An immunoconjugate having the formula of Ab-(L-D)n, wherein:
Ab is an antibody or an antigen-binding fragment thereof that specifically binds to human receptor tyrosine kinase like orphan receptor 1 (ROR1);
L is a linker;
D is an exatecan moiety or an analog thereof; and
n is an integer from 1 to 10.

2. The immunoconjugate of claim 1, wherein the linker comprises a cleavable moiety.

3. (canceled)

4. The immunoconjugate of claim 1, wherein the linker comprises a tetrapeptide GGFG (SEQ ID NO:55).

5. The immunoconjugate of claim 1, wherein the linker forms a covalent bond with a cysteine residue on the antibody or fragment.

6. The immunoconjugate of claim 1, wherein the linker is covalently bonded to the antibody or antigen-binding fragment at a succinimide, a carbonyl, a cyclooctene, a quaternised vinyl pyridine, or a triazole group of the linker.

7. (canceled)

8. (canceled)

9. The immunoconjugate of claim 8, wherein the linker comprises a phenyl ring substituted by one or more amino groups, one or more PEG chains, and/or one or more glucuronide groups.

10. The immunoconjugate of claim 1, selected from: 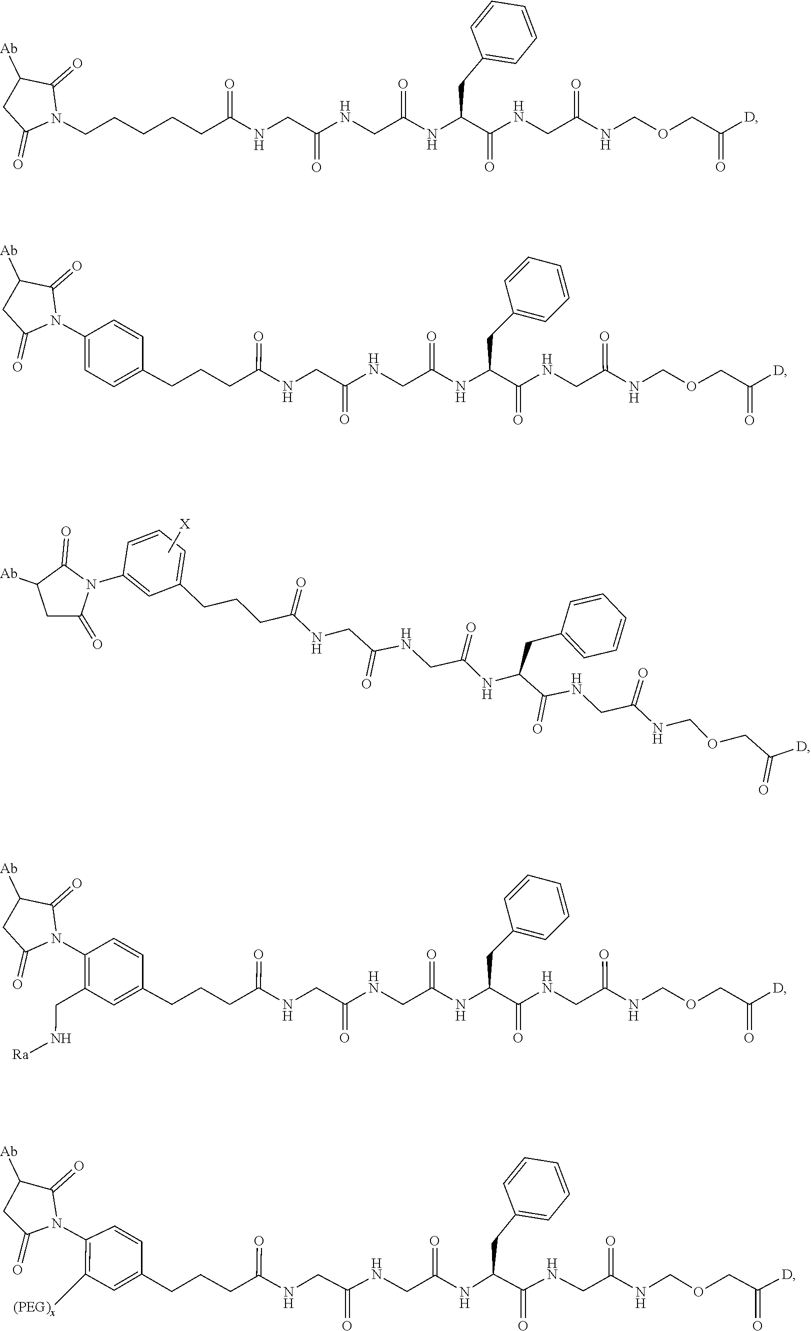 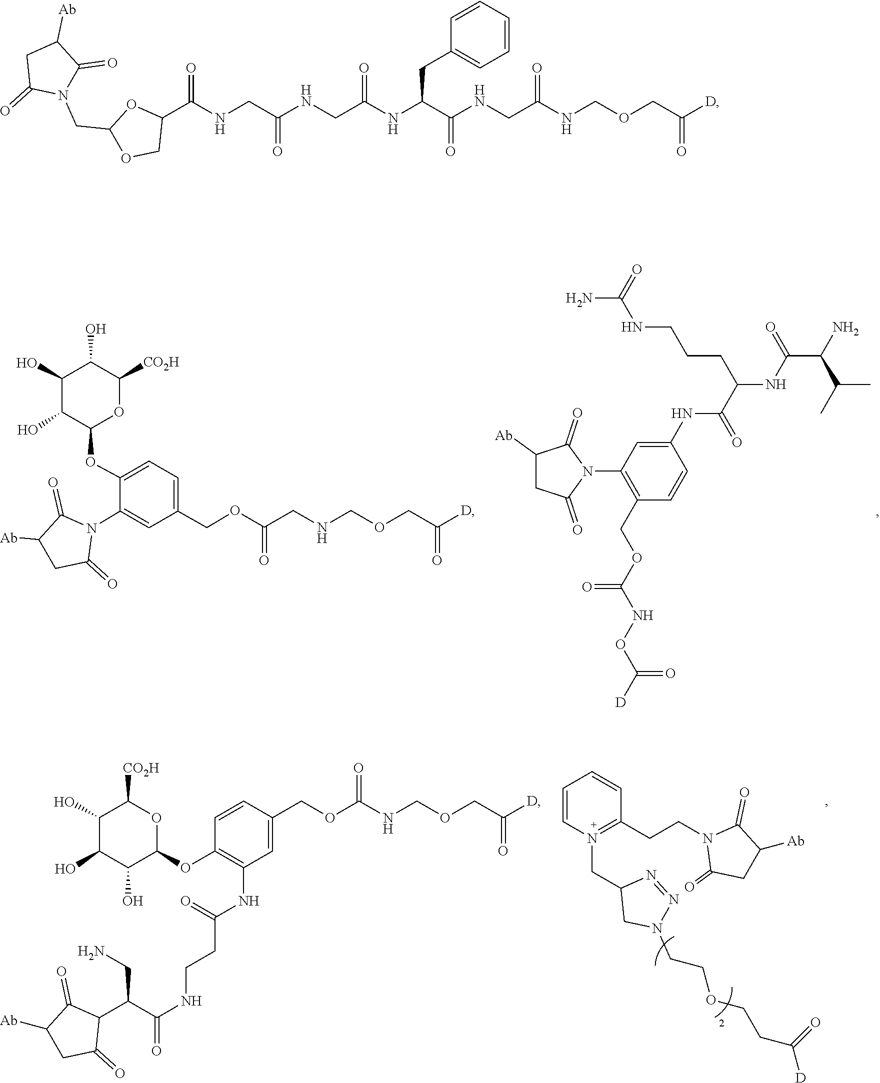 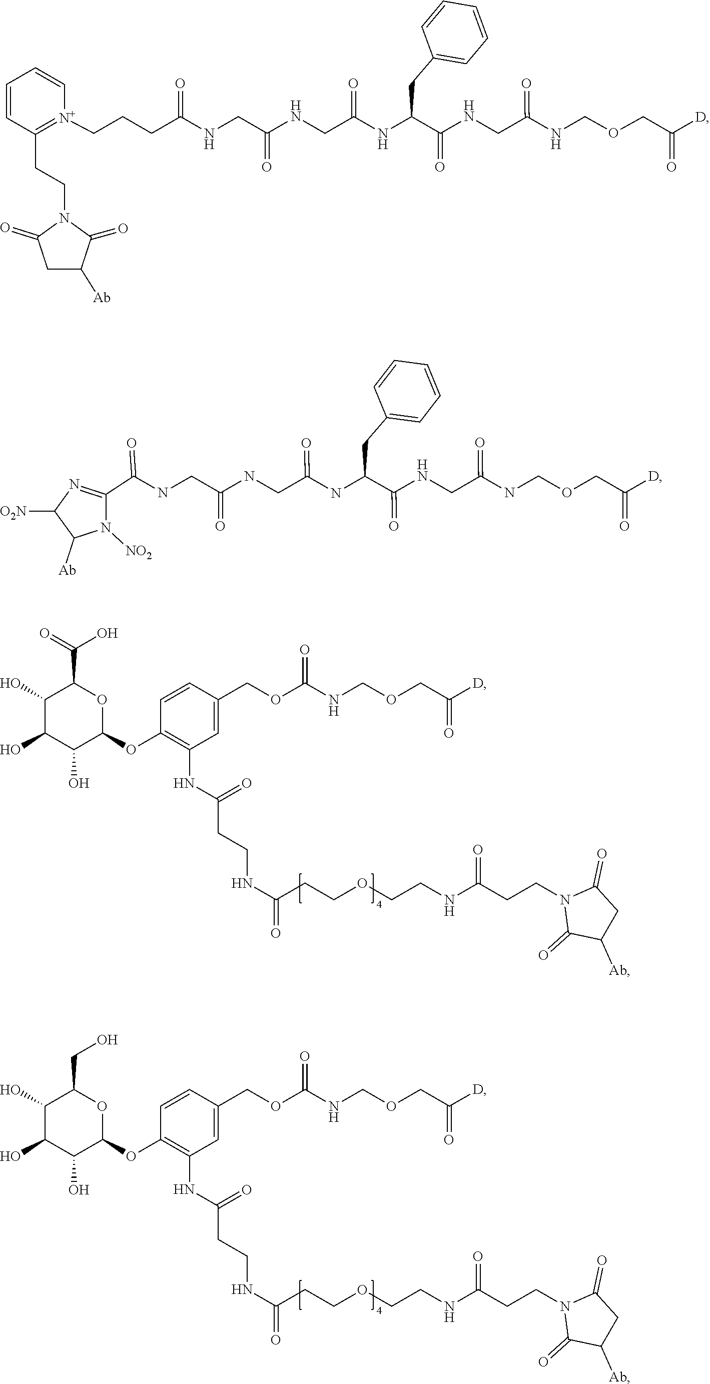 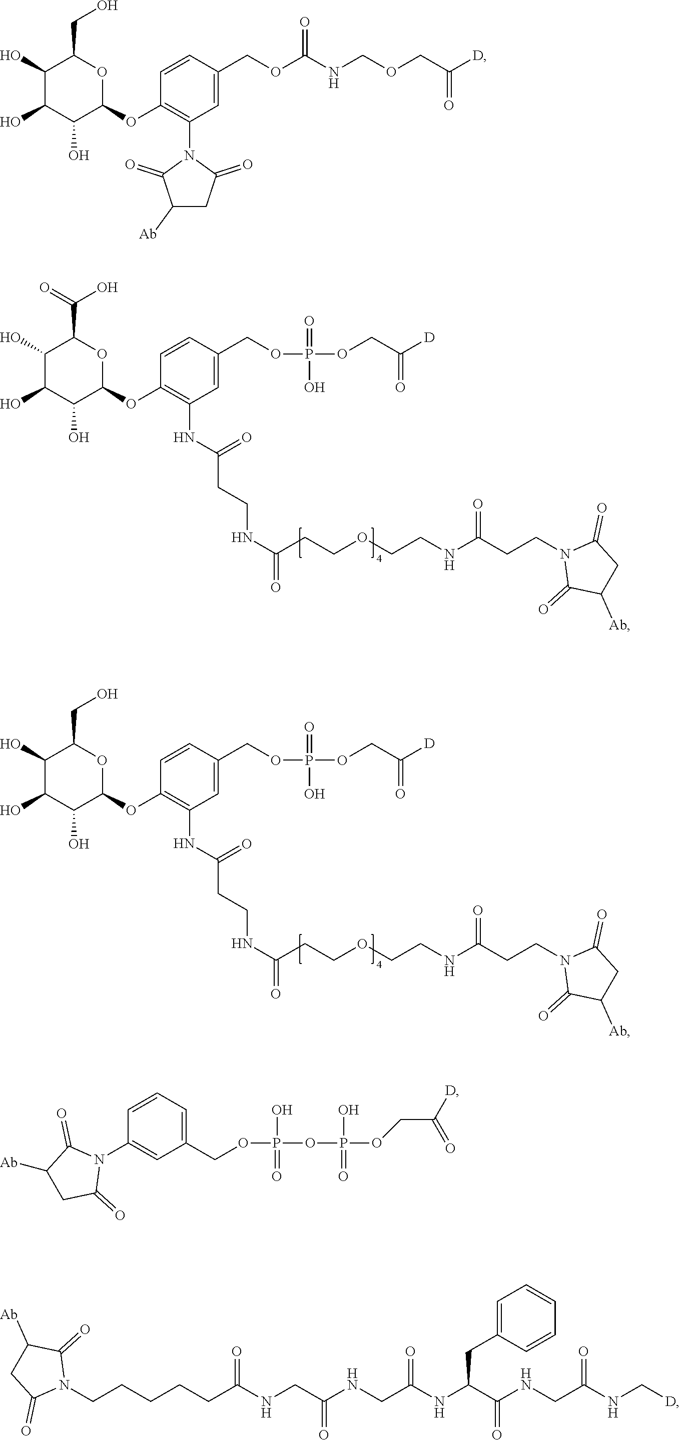 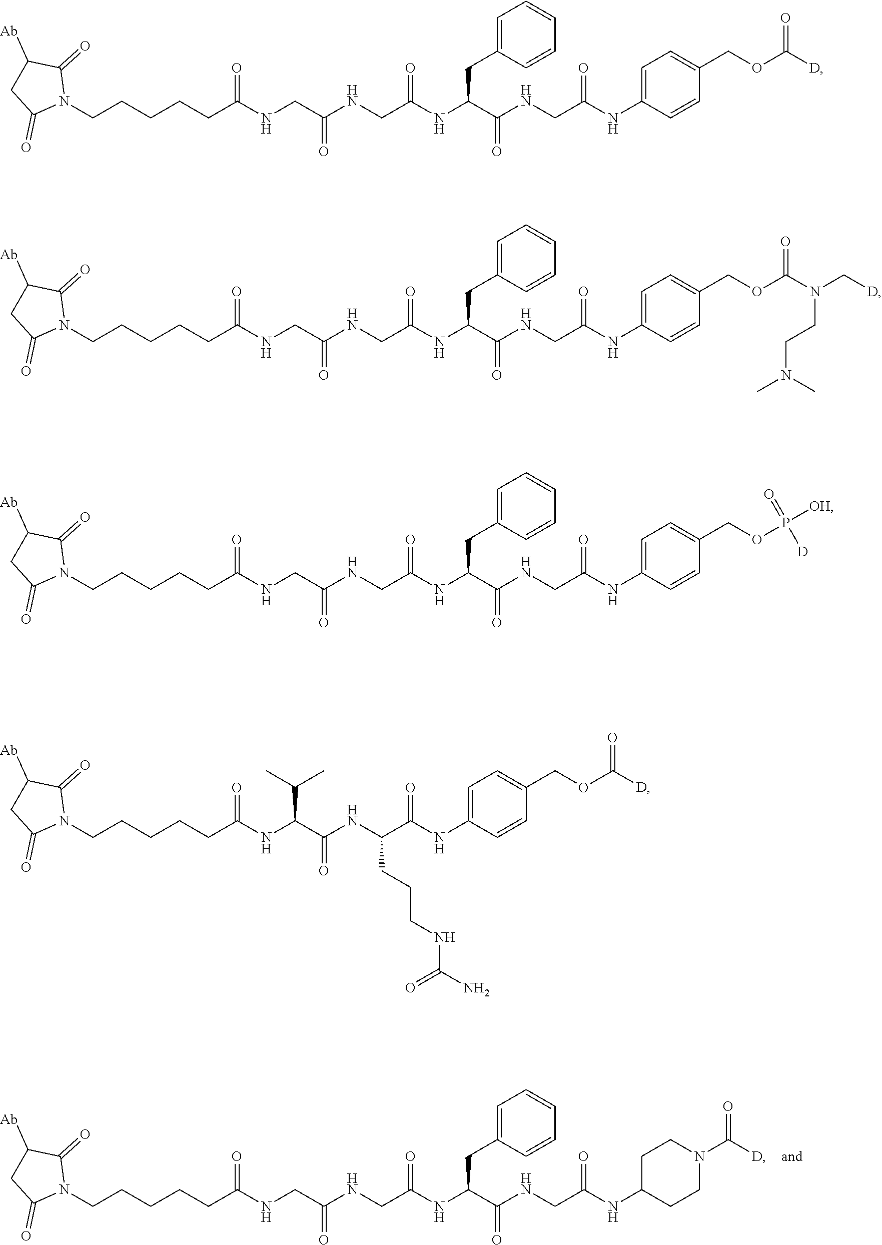 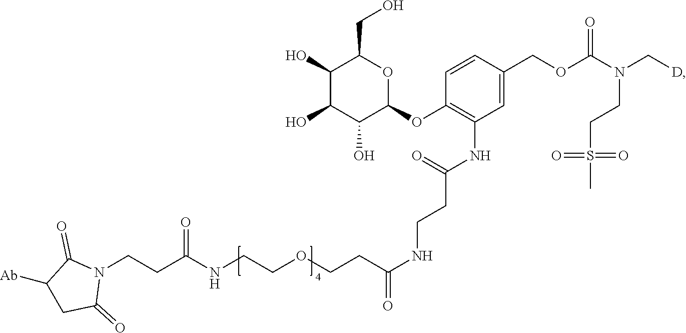
or a pharmaceutically acceptable salt thereof, wherein X is F, Cl, Br, CO₂H, SO₃H, NO₂, CF₃, or CN; Ra is H, methyl, or isopropyl; and x is an integer from 1-10.

11. The immunoconjugate of claim 1 selected from: 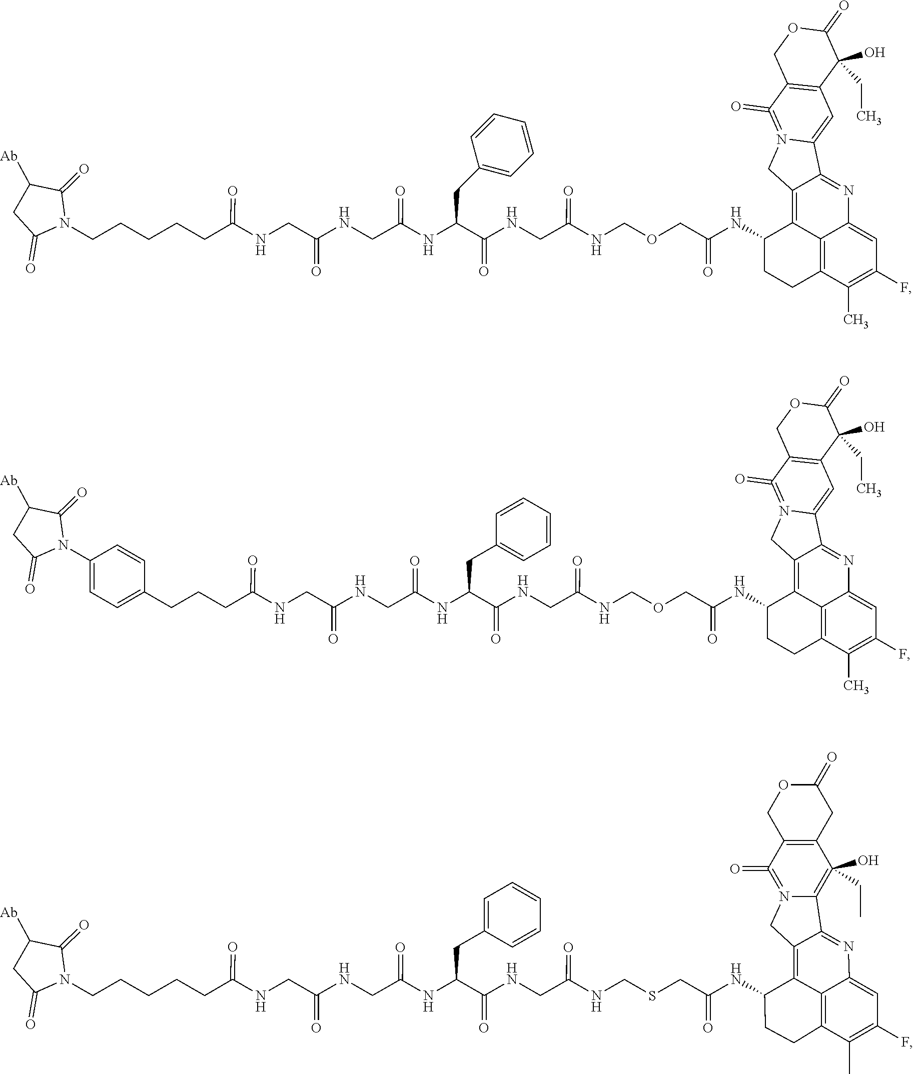 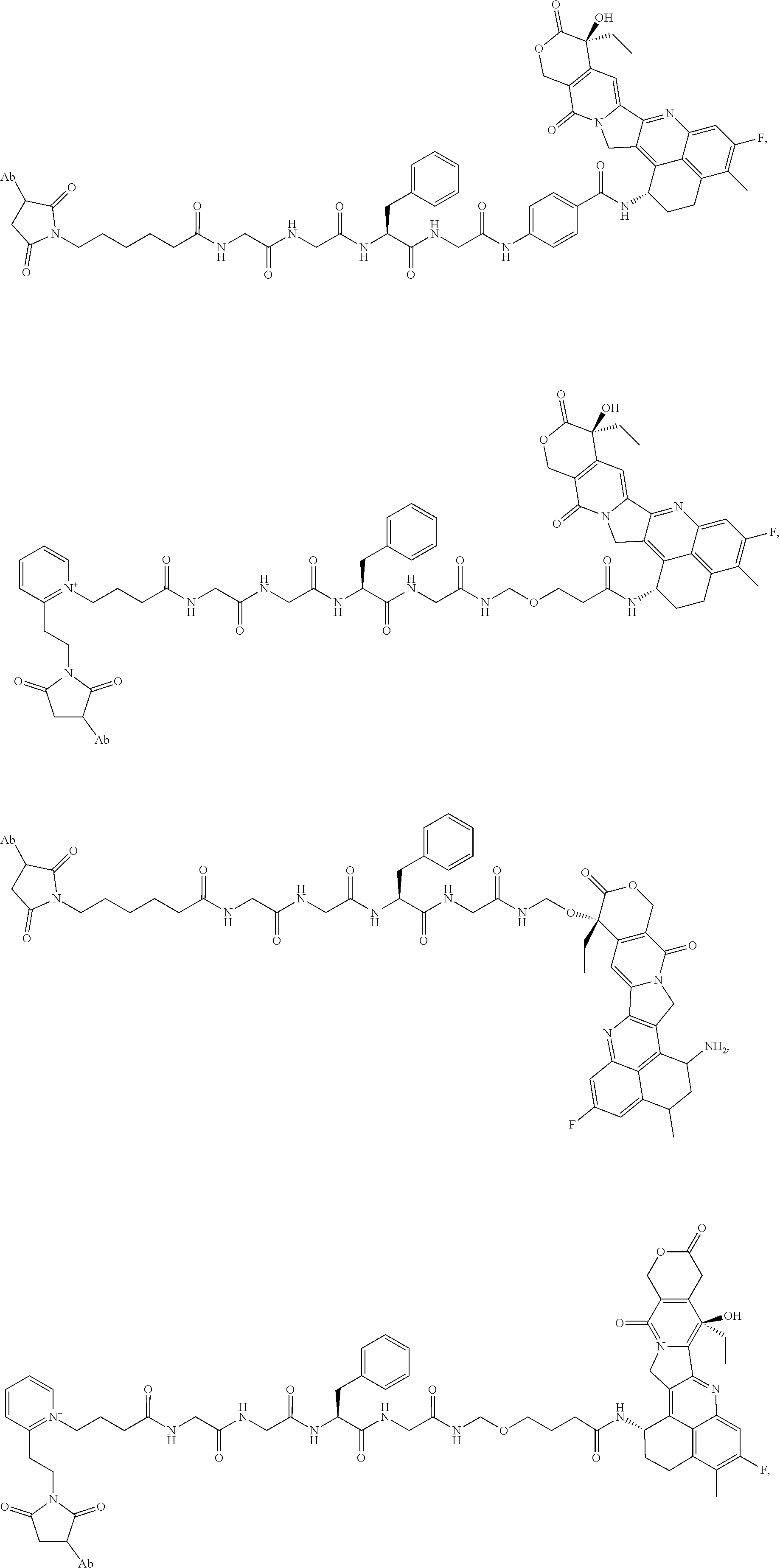 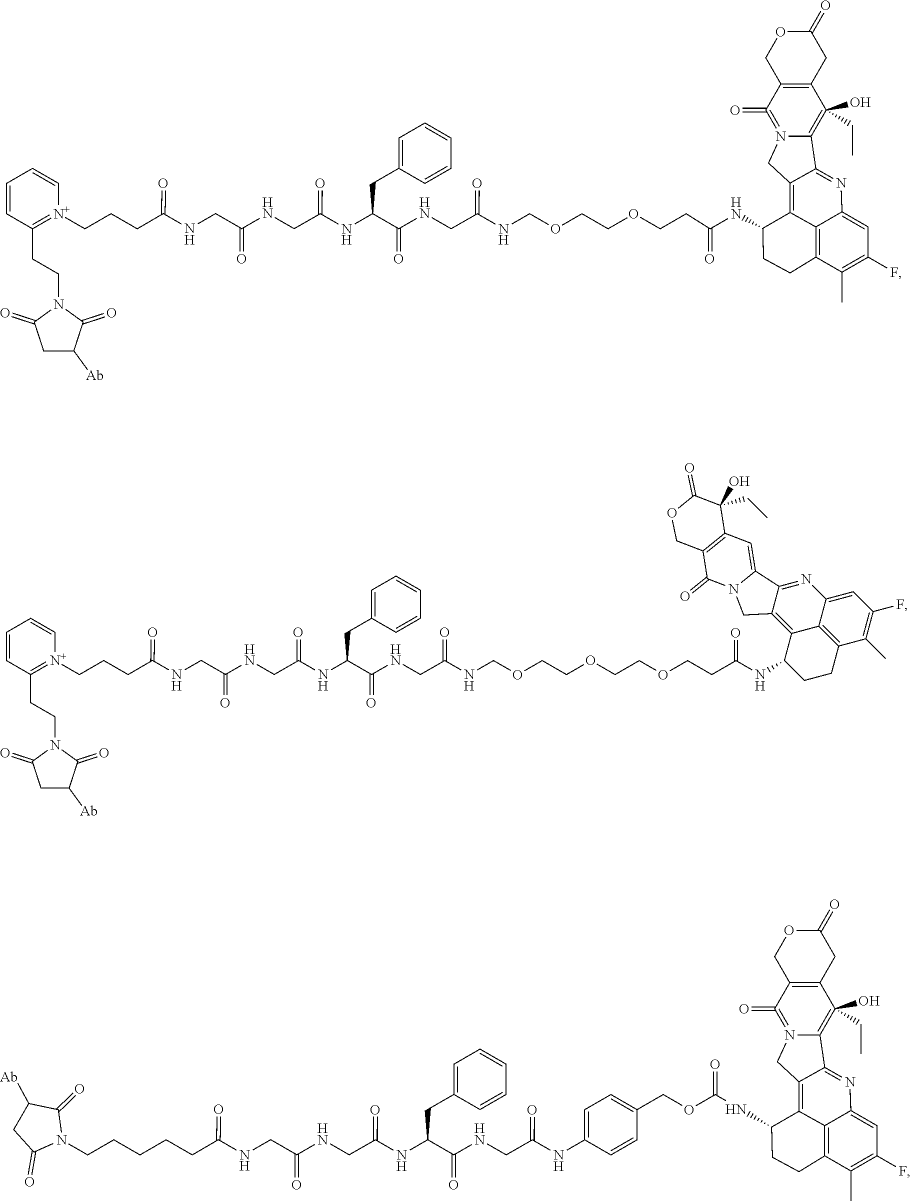 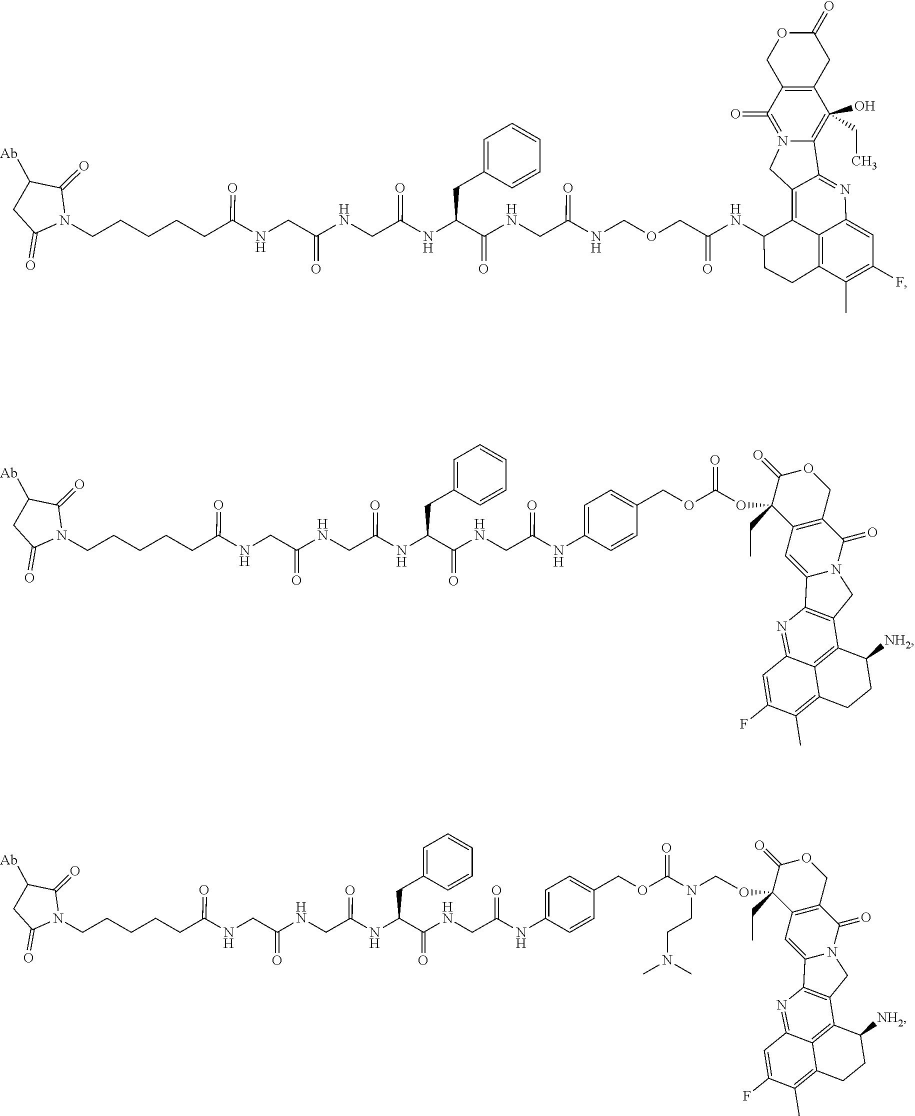 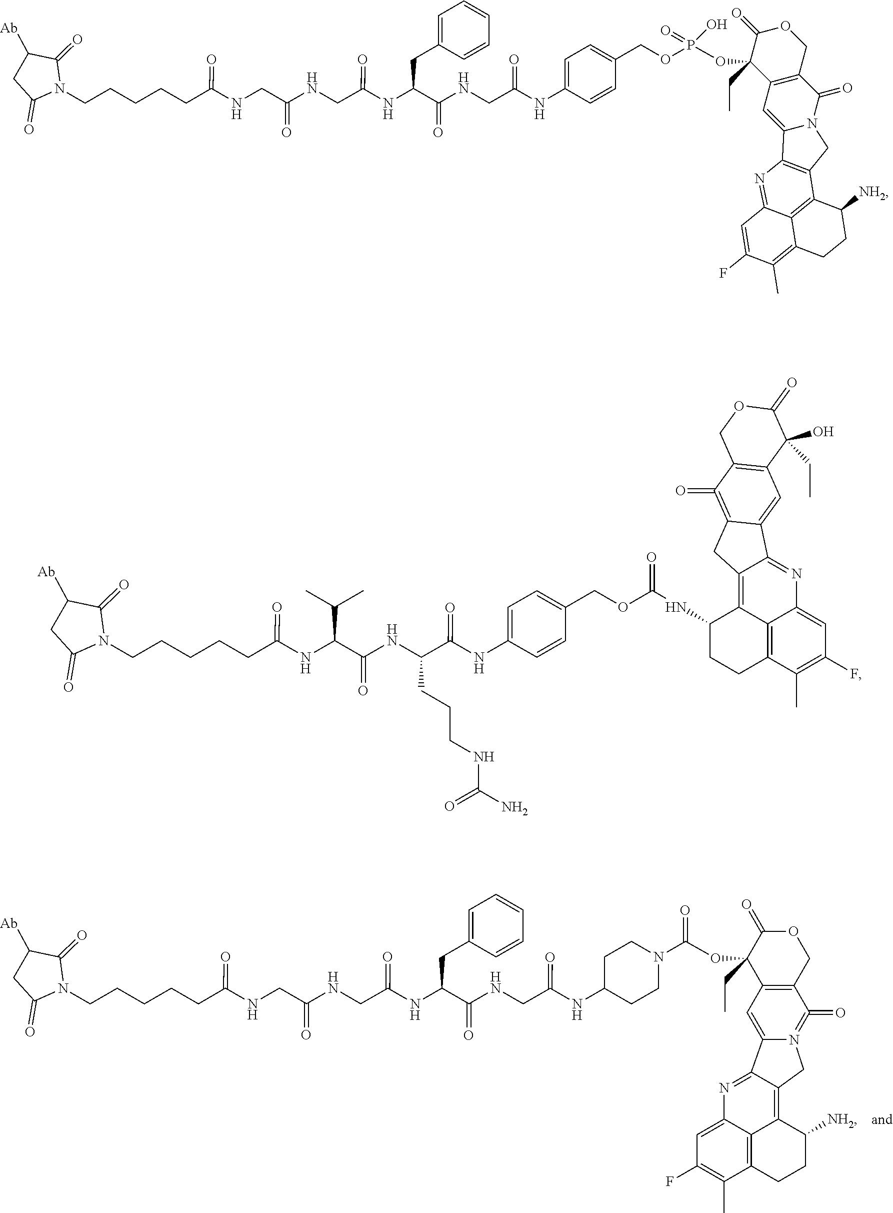 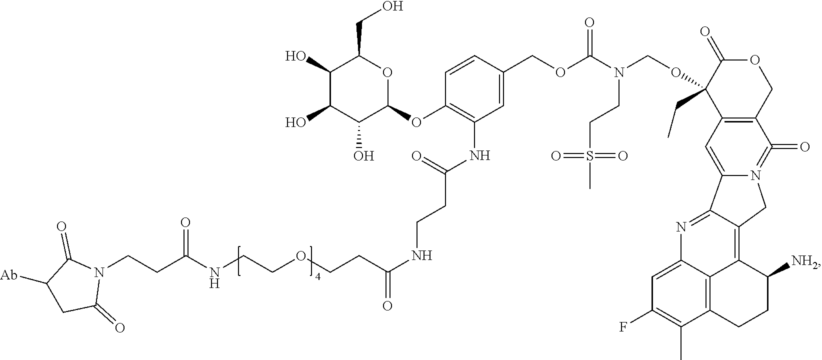
or a pharmaceutically acceptable salt thereof.

12. The immunoconjugate of claim 1, wherein the antibody or fragment binds to the same ROR1 epitope as an antibody comprising the heavy chain and light chain amino acid sequences of SEQ ID NOs: 3 and 4, respectively.

13. The immunoconjugate of claim 1, wherein the antibody comprises:
(a) the heavy chain complementarity-determining region (CDR) 1-3 (HCDR1-3) amino acid sequences in SEQ ID NO: 3 and the light chain CDR1-3 (LCDR1-3) amino sequences in SEQ ID NO: 4; or
(b) the HCDR1-3 amino acid sequences in SEQ ID NO: 69 and the LCDR1-3 amino acid sequences in SEQ ID NO: 70.

14. The immunoconjugate of claim 1, wherein
(a) the heavy chain of the antibody comprises the amino acid sequences of SEQ ID NOs: 7-9, and the light chain of the antibody comprises the amino acid sequences of SEQ ID NOs: 10⁻¹²;
(b) the heavy chain of the antibody comprises the amino acid sequences of SEQ ID NOs: 65, 8, and 9, and the light chain of the antibody comprises the amino acid sequences of SEQ ID NOs: 10⁻¹²;
(c) the heavy chain of the antibody comprises the amino acid sequences of SEQ ID NOs: 7-9, and the light chain of the antibody comprises the amino acid sequences of SEQ ID NOs: 10, 66, and 12; or
(d) the heavy chain of the antibody comprises the amino acid sequences of SEQ ID NOs: 65, 8, and 9, and the light chain of the antibody comprises the amino acid sequences of SEQ ID NOs: 10, 66, and 12.

15. (canceled)

16. The immunoconjugate of claim 15, wherein the antibody or antigen-binding fragment has one or more of the following properties:
(a) facilitates ROR1 internalization in a human cell;
(b) binds to human ROR1 with a K_{D} of less than 100 nM; and
(c) inhibits growth of ROR1⁺ human cancer cells in vitro with an EC₅₀ of 300 nM or less.

17. The immunoconjugate of claim 1, wherein the heavy chain variable domain (V_{H}) and light chain variable domain (V_{L}) of the antibody comprise the amino acid sequences of:
(a) SEQ ID NOs: 5 and 6, respectively;
(b) SEQ ID NOs: 5 and 50, respectively;
(c) SEQ ID NOs: 48 and 6, respectively;
(d) SEQ ID NOs: 48 and 50, respectively;
(e) SEQ ID NOs: 5 and 68, respectively;
(f) SEQ ID NOs: 67 and 6, respectively; or
(g) SEQ ID NOs: 67 and 68, respectively.

18. (canceled)

19. The immunoconjugate of claim 1, wherein the antibody comprises a human IgG₁ constant region, and wherein the IgG₁ constant region comprises one or more Fc region mutations selected from
(a) L234A and L235A (LALA)
(b) P329A and
(c) P329G,
wherein said Fc region mutations are identified according to the EU numbering system.

20. The immunoconjugate of claim 1, wherein the heavy chain and light chain of the antibody comprise the amino acid sequences of:
(a) SEQ ID NOs: 3 and 4, respectively;
(b) SEQ ID NOs: 3 and 49, respectively;
(c) SEQ ID NOs: 47 and 4, respectively;
(d) SEQ ID NOs: 47 and 49, respectively;
(e) SEQ ID NOs: 69 and 4, respectively;
(f) SEQ ID NOs: 3 and 70, respectively; or
(g) SEQ ID NOs: 69 and 70, respectively.

21. (canceled)

22. The immunoconjugate of claim 1, wherein the number of the drug moiety per antibody or fragment (DAR) ranges from 1 to 7.

23. An immunoconjugate comprising an anti-ROR1 antibody wherein the V_{H} and V_{L} of the antibody comprise the amino acid sequences of SEQ ID NOs: 5 and 6, respectively, and the immunoconjugate has a structure shown in Table 3 as ADC-U, -V, -W, or -X.

24. (canceled)

25. (canceled)

26. A pharmaceutical composition comprising the immunoconjugate of claim 1, and a pharmaceutically acceptable excipient.

27. A method of treating cancer in a patient in need thereof, comprising administering to the patient a therapeutically effective amount of the immunoconjugate of claim 1.

28. (canceled)

29. The method of claim 27, wherein the cancer is a leukemia, a lymphoma, or a solid tumor, optionally wherein
the cancer is chronic lymphocytic leukemia, acute lymphoblastic leukemia, acute myeloid leukemia, small lymphocytic leukemia, follicular lymphoma, T cell non-Hodgkin lymphoma, lymphoplasmacytoid lymphoma, mantle cell lymphoma, diffuse large B-cell lymphoma, Burkitt's lymphoma, Waldenström macroglobulinemia, marginal zone lymphoma, or a non-Hodgkin lymphoma that has undergone Richter's transformation, or the cancer is non-small cell lung cancer, sarcoma, ovarian cancer, or breast cancer, optionally wherein the breast cancer is triple negative breast cancer.

30. (canceled)

31. (canceled)
